(19) United States
(12) Patent Application Publication
(10) Pub. No.: US 2024/0254492 A1
(43) Pub. Date: Aug. 1, 2024

(54) MODULATION OF COASY EXPRESSION

(71) Applicants: EMPIRICO, INC., San Diego, CA (US); IONIS PHARMACEUTICALS, INC., Carlsbad, CA (US)

(72) Inventors: Omri GOTTESMAN, San Diego, CA (US); Shannon BRUSE, San Diego, CA (US); Brian CAJES, San Diego, CA (US); David JAKUBOSKY, San Diego, CA (US); Sarah KLEINSTEIN, San Diego, CA (US); John VEKICH, San Diego, CA (US); Richard LEE, Vista, CA (US); Susan F. MURRAY, Carlsbad, CA (US)

(21) Appl. No.: 18/562,221

(22) PCT Filed: May 19, 2022
(86) PCT No.: PCT/US2022/030099

§ 371 Date: Nov. 17, 2023

Related U.S. Application Data
(60) Provisional application No. 63/190,615, filed on May 19, 2021.

(51) Int. Cl. C12N 15/113; A61P 1/16

(52) U.S. Cl. CPC C12N 15/1137; A61P 1/16; C12Y 207/01024; C12N 2310/11; C12N 2310/313; C12N 2310/341; C12N 2310/351

(57) ABSTRACT

Provided herein are specific inhibitors, compositions, methods and uses for reducing expression of COASY in a cell or individual. Such specific inhibitors, compositions, and methods are useful to treat a liver disease or disorder, including but not limited to NASH, in a subject.

### SEQUENCE LISTING

The present application is being filed along with a Sequence Listing in electronic format. The Sequence Listing is provided as a file entitled BIOL0419WOSEQ_ST25.txt, created on May 12, 2022, which is 92 Kb in size. The information in the electronic format of the sequence listing is incorporated herein by reference in its entirety.

### FIELD

Provided herein are methods, antisense agents, specific inhibitors, and compositions useful for reducing expression or activity of coenzyme a synthase (COASY) in a subject. Also, provided herein are methods, specific inhibitors, and compositions which can be useful in treating COASY-related diseases or conditions in a subject. Such methods, specific inhibitors, and compositions can be useful, for example, to treat a liver disease, metabolic disease, or cardiovascular disease in a subject.

### BACKGROUND

Nonalcoholic fatty liver diseases (NAFLDs) including NASH (nonalcoholic steatohepatitis) are considered to be hepatic manifestations of the metabolic syndrome (Marchesini G, et al. Hepatology (2003) 37: 917-923) and are characterized by the accumulation of triglycerides in the liver of patients without a history of excessive alcohol consumption. The majority of patients with NAFLD are obese or morbidly obese and have accompanying insulin resistance (Byrne and Targher Hepatol (2015) 62(1S): S47-S64). The incidence of NAFLD/NASH has been rapidly increasing worldwide consistent with the increased prevalence of obesity, and is currently the most common chronic liver disease. Recently, the incidence of NAFLD and NASH was reported to be 46% and 12%, respectively, in a largely middle-aged population (Williams CD, et al. Gastroenterology (2011) 140: 124-131).

NAFLD can be broadly classified into asymptomatic simple steatosis (“fatty liver”), and NASH, in which intralobular inflammation and ballooning degeneration of hepatocytes is observed along with hepatic steatosis. The proportion of patients with NAFLD who have NASH is still not clear but might range from 20-40%. NASH is a progressive disease and can lead to liver cirrhosis and hepatocellular carcinoma (Farrell and Larter Hepatology (2006) 43: S99-S112; Cohen J C, et al. Science (2011); 332: 1519-1523). Twenty percent of NASH patients are reported to develop cirrhosis, and 30-40% of patients with NASH cirrhosis experience liver-related death (McCullough J Clin Gastroenterol (2006) 40 Suppl 1: S17-S29). Recently, NASH has become the third most common indication for liver transplantation in the United States (Charlton et al. Gastroenterology (2011) 141: 1249-1253).

Currently, the principal treatment for NAFLD and NASH is lifestyle modification by diet and exercise. However, pharmacological therapy is indispensable because some patients with NAFLD and NASH may have difficulty maintaining such improved lifestyles.

### SUMMARY

Large-scale human genetic data can improve the success rate of pharmaceutical discovery and development. A Genome Wide Association Study (GWAS) may detect associations between genetic variants and traits in a population sample. A GWAS may enable better understanding of the biology of disease and provide applicable treatments. A GWAS can utilize genotyping and/or sequencing data, and often involves an evaluation of millions of genetic variants that are relatively evenly distributed across the genome. The most common GWAS design is the case-control study, which involves comparing variant frequencies in cases versus controls. If a variant has a significantly different frequency in cases versus controls, that variant is said to be associated with disease. Association statistics that may be used in a GWAS are p-values, as a measure of statistical significance; odds ratios (OR), as a measure of effect size; or beta coefficients (beta), as a measure of effect size. Researchers often assume an additive genetic model and calculate an allelic odds ratio, which is the increased (or decreased) risk of disease conferred by each additional copy of an allele (compared to carrying no copies of that allele). An additional concept in design and interpretation of GWAS is that of linkage disequilibrium, which is the non-random association of alleles. The presence of linkage disequilibrium can obfuscate which variant is “causal.”

Functional annotation of variants and/or wet lab experimentation can identify the causal genetic variant identified via GWAS, and in many cases may lead to the identification of disease-causing genes. In particular, understanding the functional effect of a causal genetic variant (for example, loss of protein function, gain of protein function, increase in gene expression, or decrease in gene expression) may allow that variant to be used as a proxy for therapeutic modulation of the target gene, or to gain insight into potential therapeutic efficacy and safety of a therapeutic that modulates that target.

Identification of such gene-disease associations has provided insights into disease biology and may be used to identify novel therapeutic targets for the pharmaceutical industry. In order to translate the therapeutic insights derived from human genetics, disease biology in patients may be exogenously ‘programmed’ into replicating the observation from human genetics. There are several potential options for therapeutic modalities that may be brought to bear in translating therapeutic targets identified via human genetics into novel medicines. These may include well established therapeutic modalities such as small molecules and monoclonal antibodies, maturing modalities such as oligonucleotides, and emerging modalities such as gene therapy and gene editing. The choice of therapeutic modality can depend on several factors including the location of a target (for example, intracellular, extracellular, or secreted), a relevant tissue (for example, lung or liver) and a relevant indication.

The COASY gene is located on chromosome 17 in humans and encodes the coenzyme A synthase (COASY protein), a mitochondrial bi-functional enzyme that has two catalytic domains, phosphopantetheine adenylyltransferase (PPAT) and dephospho-CoA kinase (DPCK) and is activated by phospholipids. The COASY protein mediates the final two stages of de novo coenzyme A (CoA) synthesis from pantothenic acid in mammalian cells. CoA and its derivatives are involved in multiple cellular metabolic pathways including pyruvate oxidation, fatty acid synthesis, cell cycle progression and cell death. Some further details relevant to a COASY protein may be found at UniProt.org under accession no. Q13057 (last modified Feb. 23, 2022). The COASY protein typically includes 564 amino acid, but at least one other isoform has been described, as provided at UniProt.org under the aforementioned accession number. Here, it is shown that genetic variants that cause inactivation of the COASY gene in humans are associated with decreased risk of NAFLD and reduced liver fat percentages.

Provided herein are compositions, specific inhibitors and methods for modulating expression of COASY. In certain embodiments, the COASY-specific inhibitor decreases expression or activity of COASY. In certain embodiments, COASY-specific inhibitors include antisense agents, proteins and small molecules. In certain embodiments, the COASY-specific inhibitor is an antisense agent. In certain embodiments, the COASY-specific inhibitor comprises a modified oligonucleotide. In certain embodiments, the antisense agent can be single stranded or double stranded.

Certain embodiments are directed to compounds useful for inhibiting COASY, which can be useful for treating a liver disease, metabolic disease, or cardiovascular disease. Certain embodiments relate to the novel findings of antisense inhibition of COASY resulting in improvement of symptoms or endpoints associated with a liver disease, metabolic disease, or cardiovascular disease. Certain embodiments are directed to COASY-specific inhibitors useful in improving hepatic steatosis, liver fibrosis, triglyceride synthesis, plasma lipid levels, hepatic lipids, ALT levels, NAFLD Activity Score (NAS), or plasma cholesterol levels, or a combination thereof.

Certain embodiments are described in the numbered embodiments below: Embodiment 1. A method of treating a liver disease or disorder in a subject having a liver disease or disorder, comprising administering a COASY-specific inhibitor to the subject, thereby treating the liver disease or disorder in the subject.

Embodiment 2. The method of embodiment 1, wherein the liver disease or disorder is fatty liver disease, chronic liver disease, liver cirrhosis, hepatic steatosis, steatohepatitis, nonalcoholic fatty liver disease (NAFLD), alcoholic steatohepatitis (ASH), or nonalcoholic steatohepatitis (NASH).

Embodiment 3. A method comprising administering a COASY-specific inhibitor to a subject.

Embodiment 4. The method of embodiment 3, wherein the subject has a liver disease or is at risk for developing a liver disease.

Embodiment 5. The method of embodiment 4, wherein the the liver disease or disorder is fatty liver disease, chronic liver disease, liver cirrhosis, hepatic steatosis, steatohepatitis, NAFLD, ASH, or NASH.

Embodiment 6. The method of any of embodiments 1-5, wherein a therapeutic amount of the COASY-specific inhibitor is administered to the subject.

Embodiment 7. The method of any of embodiments 1-6, wherein a therapeutic amount of the COASY-specific inhibitor ameliorates at least one symptom of the liver disease.

Embodiment 8. The method of any of embodiments 1-7, wherein the administration of the COASY-specific inhibitor ameliorates at least one symptom of fatty liver disease, chronic liver disease, liver cirrhosis, hepatic steatosis, steatohepatitis, NAFLD, ASH, or NASH.

Embodiment 9. The method of embodiment 8, wherein the at least one symptom is hepatic steatosis, liver fibrosis, elevated triglyceride level, elevated plasma lipid level, elevated hepatic lipid level, elevated ALT level, high NAFLD Activity score, or elevated plasma cholesterol level.

Embodiment 10. The method of any of embodiments 1-9, wherein administering the COASY-specific inhibitor reduces hepatic steatosis, reduces liver fibrosis, reduces plasma lipid levels, reduces plasma triglyceride levels, reduces plasma cholesterol levels, reduces ALT levels, improves NAS, reduces hepatic lipid levels, reduces hepatic triglyceride levels, or reduces hepatic cholesterol levels in the subject, or a combination thereof.

Embodiment 11. The method of any of embodiments 1-10, wherein the COASY-specific inhibitor reduces levels of hydroxyproline, reduces levels of Collal, reduces levels of ORO, or reduces levels total collagen in the liver of the subject, or a combination thereof.

Embodiment 12. The method of any of embodiments 1-11, wherein the subject is a human subject.

Embodiment 13. A method comprising contacting a cell with a COASY-specific inhibitor.

Embodiment 14. The method of embodiment 13, wherein expression of COASY in the cell is reduced.

Embodiment 15. A method of inhibiting expression or activity of COASY in a cell comprising contacting the cell with a COASY-specific inhibitor, thereby inhibiting expression or activity of COASY in the cell.

Embodiment 16. The method of any of embodiments 13-15, wherein the cell is a hepatocyte.

Embodiment 17. The method of any of embodiments 13-16, wherein the cell is in a subject.

Embodiment 18. The method of embodiment 17, wherein the subject has, or is at risk of having liver disease, fatty liver disease, chronic liver disease, liver cirrhosis, hepatic steatosis, steatohepatitis, NAFLD, ASH, or NASH.

Embodiment 19. The method of any of embodiments 1-8, wherein the COASY-specific inhibitor is an antisense agent, a polypeptide, an antibody, or a small molecule.

Embodiment 20. The method of any of embodiments 1-19, wherein the COASY-specific inhibitor is an antisense agent comprising a modified oligonucleotide, wherein the modified oligonucleotide has a nucleobase sequence complementary to the nucleobase sequence of a COASY nucleic acid.

Embodiment 21. The method of any of embodiments 1-20, wherein the nucleobase sequence of the modified oligonucleotide is complementary to any of SEQ ID NOs: 1-4.

Embodiment 22. The method of embodiment 21, wherein the nucleobase sequence modified oligonucleotide is complementary to SEQ ID NO: 3 or SEQ ID NO: 4.

Embodiment 23. The method of embodiment 22, wherein the nucleobase sequence of the modified oligonucleotide is at least 90% complementary to an equal length portion of SEQ ID NO: 3 or SEQ ID NO: 4.

Embodiment 24. The method of embodiment 22, wherein the nucleobase sequence of the modified oligonucleotide is at least 95% complementary to an equal length portion of SEQ ID NO: 3 or SEQ ID NO: 4.

Embodiment 25. The method of embodiment 22, wherein the nucleobase sequence of the modified oligonucleotide is 100% complementary to an equal length portion of SEQ ID NO: 3 or SEQ ID NO: 4.

Embodiment 26. The method of any of embodiments 20-25, wherein at least one nucleoside of the modified oligonucleotide comprises a modified sugar moiety.

Embodiment 27. The method of embodiment 26, wherein the modified sugar moiety comprises a bicyclic sugar moiety.

Embodiment 28. The method of embodiment 27, wherein the bicyclic sugar moiety comprises a 4′—CH(CH₃)—O-2′ bridge or a 4′—(CH₂)ₙ—O-2′ bridge, wherein n is 1 or 2.

Embodiment 29. The method of embodiment 26, wherein the modified sugar moiety comprises a non-bicyclic modified sugar moiety.

Embodiment 30. The method of embodiment 29, wherein the non-bicyclic sugar moiety is a 2′-F, 2′-OMe, or 2′-MOE sugar moiety.

Embodiment 31. The method of any of embodiments 20-30, wherein the antisense agent is single-stranded.

Embodiment 32. The method of any of embodiments 20-30, wherein the antisense agent is double-stranded.

Embodiment 33. The method of any of embodiments 20-32, wherein the modified oligonucleotide consists of 12 to 30 linked nucleosides.

Embodiment 34. The method of any of embodiments 20-33, wherein at least one nucleoside of the modified oligonucleotide comprises a modified nucleobase.

Embodiment 35. The method of embodiment 34, wherein the modified nucleobase is 5-methylcytosine.

Embodiment 36. The method of any of embodiments 20-35, wherein at least one internucleoside linkage of the modified oligonucleotide is a modified internucleoside linkage.

Embodiment 37. The method of embodiment 36, wherein the at least one modified internucleoside linkage is a phosphorothioate internucleoside linkage.

Embodiment 38. The method of embodiment 36, wherein each internucleoside linkage is a phosphorothioate internucleoside linkage.

Embodiment 39. The method of embodiment 36, wherein each internucleoside linkage is independently selected from a phosphodiester internucleoside linkage and a phosphorothioate internucleoside linkage.

Embodiment 40. The method of any one of embodiments 20-39, wherein the modified oligonucleotide has:
a gap segment consisting of linked 2′-deoxynucleosides;
a 5′ wing segment consisting of linked nucleosides;
a 3′ wing segment consisting linked nucleosides;
wherein the gap segment is positioned immediately adjacent to and between the 5′ wing segment and the 3′
wing segment and wherein each nucleoside of each wing segment comprises a modified sugar.

Embodiment 41. The method of embodiment 40 wherein the gap segment is positioned immediately adjacent to and between the 5′ wing segment and the 3′ wing segment and wherein each nucleoside of each wing segment comprises a modified sugar moiety.

Embodiment 42. The method of any of embodiments 20-41, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides;
wherein the 3′-most nucleoside of the 5′-region and the 5′-most nucleoside of the 3′-region comprise modified sugar moieties, and
each of the central region nucleosides is selected from a nucleoside comprising a 2′-β-D-deoxyribosyl sugar moiety and a nucleoside comprising a 2′-substituted sugar moiety, wherein the central region comprises at
least six nucleosides comprising a 2′-β-D-deoxyribosyl sugar moiety and no more than two nucleosides comprise a 2′-substituted sugar moiety.

Embodiment 43. The method of any of embodiment 1-42, wherein the COASY-specific inhibitor is administered parenterally.

Embodiment 44. The method of embodiment 43, wherein the COASY-specific inhibitor is administered parenterally by subcutaneous or intravenous administration.

Embodiment 45. The method of any of embodiments 1-44, comprising co-administering the COASY-specific inhibitor and at least one additional therapy.

Embodiment 46. The method of any of embodiments 20-45, wherein the antisense agent comprises a conjugate group.

Embodiment 47. The method of embodiment 46, wherein the conjugate group comprises N-acetyl galactosamine.

Embodiment 48. The method of any of embodiments 1-47, wherein the COASY-specific inhibitor is an RNase H agent capable of reducing the amount of COASY nucleic acid through the activation of RNase H.

Embodiment 49. The method of any of embodiments 1-47, wherein the COASY-specific inhibitor is an RNAi agent capable of reducing the amount of COASY nucleic acid through the activation of RISC/Ago2.

Embodiment 50. The method of any of embodiments 1-47, wherein the COASY-specific inhibitor is a steric-blocking agent capable of directly binding to a target nucleic acid, thereby blocking the interaction of the COASY nucleic acid with other nucleic acids or proteins.

Embodiment 51. Use of a COASY-specific inhibitor for the manufacture or preparation of a medicament for treating a liver disease or disorder.

Embodiment 52. Use of a COASY-specific inhibitor for the treatment of a liver disease or disorder.

Embodiment 53. The use of embodiment 51 or 52, wherein the liver disease or disorder is fatty liver disease, chronic liver disease, liver cirrhosis, hepatic steatosis, steatohepatitis, NAFLD, ASH, or NASH.

Embodiment 54. The use of any of embodiments 51-53, wherein the COASY-specific inhibitor reduces or improves hepatic steatosis, liver fibrosis, plasma lipid levels, plasma triglyceride levels, plasma cholesterol levels, ALT levels, NAFLD Activity Score (NAS), hepatic lipid levels, hepatic triglyceride levels, or hepatic cholesterol levels, or a combination thereof.

Embodiment 55. The use of any of embodiment 51-54, wherein the COASY-specific inhibitor reduces hepatic steatosis, reduces liver fibrosis, reduces plasma lipid levels, reduces plasma triglyceride levels, reduces plasma cholesterol levels, reduces ALT levels, improves NAS, reduces hepatic lipid levels, reduces hepatic triglyceride levels, or reduces hepatic cholesterol levels, or a combination thereof.

Embodiment 56. The use of any of embodiments 51-55, wherein the COASY-specific inhibitor reduces levels of hydroxyproline, reduces levels of Collal, reduces levels of ORO, or reduces levels total collagen in the liver, or a combination thereof.

Embodiment 57. The use of any of embodiments 51-56, wherein the COASY-specific inhibitor is an antisense agent, a polypeptide, an antibody, or a small molecule.

Embodiment 58. The use of any of embodiments 51-57, wherein the COASY-specific inhibitor is an antisense agent comprising a modified oligonucleotide, wherein the modified oligonucleotide has a nucleobase sequence complementary to the nucleobase sequence of a COASY nucleic acid.

Embodiment 59. The use of embodiment 58, wherein the nucleobase sequence of the modified oligonucleotide is complementary to any of SEQ ID NOs: 1-4.

Embodiment 60. The use of embodiment 58, wherein the nucleobase sequence modified oligonucleotide is complementary to SEQ ID NO: 3 or SEQ ID NO: 4.

Embodiment 61. The use of embodiment 58, wherein the nucleobase sequence of the modified oligonucleotide is at least 90% complementary to an equal length portion of SEQ ID NO: 3 or SEQ ID NO: 4.

Embodiment 62. The use of embodiment 58, wherein the nucleobase sequence of the modified oligonucleotide is at least 95% complementary to an equal length portion of SEQ ID NO: 3 or SEQ ID NO: 4.

Embodiment 63. The use of embodiment 58, wherein the nucleobase sequence of the modified oligonucleotide is 100% complementary to an equal length portion of SEQ ID NO: 3 or SEQ ID NO: 4.

Embodiment 64. The use of any of embodiments 58-63, wherein at least one nucleoside of the modified oligonucleotide comprises a modified sugar moiety.

Embodiment 65. The use of embodiment 64, wherein the modified sugar moiety comprises a bicyclic sugar moiety.

Embodiment 66. The use of embodiment 65, wherein the bicyclic sugar moiety comprises a 4′—CH(CH₃)—O-2′ bridge or a 4′—(CH₂)ₙ—O-2′ bridge, wherein n is 1 or 2.

Embodiment 67. The use of embodiment 64, wherein the modified sugar moiety comprises a non-bicyclic modified sugar moiety.

Embodiment 68. The use of embodiment 67, wherein the non-bicyclic sugar moiety is a 2′-F, 2′-OMe, or 2′-MOE sugar moiety.

Embodiment 69. The use of any of embodiments 58-68, wherein the antisense agent is single-stranded.

Embodiment 70. The use of any of embodiments 58-68, wherein the antisense agent is double-stranded.

Embodiment 71. The use of any of embodiments 58-70, wherein the modified oligonucleotide consists of 12 to 30 linked nucleosides.

Embodiment 72. The use of any of embodiments 58-71, wherein at least one nucleoside of the modified oligonucleotide comprises a modified nucleobase.

Embodiment 73. The use of embodiment 72, wherein the modified nucleobase is 5-methylcytosine.

Embodiment 74. The use of any of embodiments 58-73, wherein at least one internucleoside linkage of the modified oligonucleotide is a modified internucleoside linkage.

Embodiment 75. The use of embodiment 74, wherein the at least one modified internucleoside linkage is a phosphorothioate internucleoside linkage.

Embodiment 76. The use of embodiment 74, wherein each internucleoside linkage is a phosphorothioate internucleoside linkage.

Embodiment 77. The use of embodiment 74, wherein each internucleoside linkage is independently selected from a phosphodiester internucleoside linkage and a phosphorothioate internucleoside linkage.

Embodiment 78. The use of any one of embodiments 58-77, wherein the modified oligonucleotide has:
a gap segment consisting of linked 2′-deoxynucleosides;
a 5′ wing segment consisting of linked nucleosides;
a 3′ wing segment consisting linked nucleosides;
wherein the gap segment is positioned immediately adjacent to and between the 5′ wing segment and the 3′ wing segment and wherein each nucleoside of each wing segment comprises a modified sugar moiety.

Embodiment 79. The use of embodiment 78 wherein the gap segment is positioned immediately adjacent to and between the 5′ wing segment and the 3′ wing segment and wherein each nucleoside of each wing segment comprises a modified sugar moiety.

Embodiment 80. The use of any of embodiments 58-79, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides;
wherein the 3′-most nucleoside of the 5′-region and the 5′-most nucleoside of the 3′-region comprise
modified sugar moieties, and each of the central region nucleosides is selected from a nucleoside comprising a 2′-β-D-deoxyribosyl sugar moiety and a nucleoside comprising a 2′-substituted sugar moiety, wherein the central region comprises at least six nucleosides comprising a 2′-β-D-deoxyribosyl sugar moiety and no more than two nucleosides comprise a 2′-substituted sugar moiety.

Embodiment 81. The use of any of embodiments 51-81, wherein the COASY-specific inhibitor is administered parenterally.

Embodiment 82. The use of embodiment 81, wherein the COASY-specific inhibitor is administered parenterally by subcutaneous or intravenous administration.

Embodiment 83. The use of any of embodiments 51-82, comprising co-administering the COASY-specific inhibitor and at least one additional therapy.

Embodiment 84. The use of any of embodiments 58-83, wherein the antisense agent comprises a conjugate group.

Embodiment 85. The use of embodiment 84, wherein the conjugate group comprises N-acetyl galactosamine.

Embodiment 86. The use of any of embodiments 51-85, wherein the COASY-specific inhibitor is an RNase H agent capable of reducing the amount of COASY nucleic acid through the activation of RNase H.

Embodiment 87. The use of any of embodiments 51-85, wherein the COASY-specific inhibitor is an RNAi agent capable of reducing the amount of COASY nucleic acid through the activation of RISC/Ago2.

Embodiment 88. The use of any of embodiments 51-85, wherein the COASY-specific inhibitor is a steric-blocking agent capable of directly binding to a target nucleic acid, thereby blocking the interaction of the COASY nucleic acid with other nucleic acids or proteins.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO in the examples contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Compounds described by ISIS/IONIS number (ISIS/ION #) indicate a combination of nucleobase sequence, chemical modification, and motif.

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

“2′-deoxynucleoside” means a nucleoside comprising a 2′-H(H) furanosyl sugar moiety, as found in naturally occurring deoxyribonucleic acids (DNA). In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside may comprise a modified nucleobase or may comprise an RNA nucleobase (uracil).

“2′-MOE” means a 2′-OCH₂CH₂OCH₃ group in place of the 2′—OH group of a furanosyl sugar moiety. A “2′-MOE sugar moiety” or a “2′-MOE modified sugar moiety” means a sugar moiety with a 2′-OCH₂CH₂OCH₃ group in place of the 2′—OH group of a furanosyl sugar moiety. Unless otherwise indicated, a 2′-MOE sugar moiety is in the β-D-ribosyl configuration. “MOE” means O-methoxyethyl. “2′-MOE nucleoside” (also 2′-O-methoxyethyl nucleoside) means a nucleoside comprising a 2′-MOE sugar moiety.

“2′-OMe” means a 2′-OCH₃ group in place of the 2′—OH group of a furanosyl sugar moiety. A “2′-O-methyl sugar moiety” or “2′-OMe sugar moiety” or a “2′-OMe modified sugar moiety” means a sugar moiety with a 2′-OCH₃ group in place of the 2′—OH group of a furanosyl sugar moiety. Unless otherwise indicated, a 2′-MOE sugar moiety is in the β-D-ribosyl configuration.

As used herein, “2′-OMe nucleoside” means a nucleoside comprising a 2′-OMe sugar moiety. As used herein, “2′-F” means a 2′-fluoro group in place of the 2′—OH group of a ribosyl sugar moiety. A “2′-F sugar moiety” or “2′-fluororibosyl sugar moiety” means a sugar moiety with a 2′—F group in place of the 2′—OH group of a ribosyl sugar moiety. Unless otherwise indicated, a 2′-F has the β-D ribosyl stereochemical configuration.

As used herein, “2′-F nucleoside” means a nucleoside comprising a 2′-F sugar moiety.

“2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.

“3′ target site” refers to the nucleotide of a target nucleic acid which is complementary to the 3′-most nucleotide of a particular compound.

“5′ target site” refers to the nucleotide of a target nucleic acid which is complementary to the 5′-most nucleotide of a particular compound.

“5-methylcytosine” means a cytosine with a methyl group attached to the 5 position. A 5-methylcytosine is a modified nucleobase.

“About” means within 10% of a value. For example, if it is stated, “the compounds affected about 70% inhibition of COASY,” it is implied that COASY levels are inhibited within a range of 60% and 80%.

As used herein, “administration” or “administering” means providing a pharmaceutical agent or composition to an animal.

“Administered concomitantly” or “co-administration” means administration of two or more compounds in any manner in which the pharmacological effects of both are manifest in the patient. Concomitant administration does not require that both compounds be administered in a single pharmaceutical composition, in the same dosage form, by the same route of administration, or at the same time. The effects of both compounds need not manifest themselves at the same time. The effects need only be overlapping for a period of time and need not be coextensive. Concomitant administration or co-administration encompasses administration in parallel or sequentially.

“Ameliorate” or “amelioration” in reference to a treatment means improvement or lessening of at least one indicator, sign, or symptom of an associated disease, disorder, or condition relative to the same indicator, sign, or symptom in the absence of the treatment. In certain embodiments, amelioration is the reduction in the severity or frequency of a symptom or the delayed onset of slowing of progression in the severity or frequency of a symptom. In certain embodiments, the symptom is hepatic steatosis, liver fibrosis, elevated triglyceride synthesis, elevated plasma lipid levels, elevated hepatic lipids, elevated ALT levels, high NAFLD Activity Score (NAS), or elevated plasma cholesterol levels, in a subject, or a combination thereof. In certain embodiments, the symptom is elevated levels of hydroxyproline, elevated levels of Collal, elevated levels of ORO, or elevated levels of total collagen in the liver of a subject, or a combination thereof. The progression or severity of indicators may be determined by subjective or objective measures, which are known to those skilled in the art. For example, NAS may be determined at least as described in Kleiner, et. al., Hepatology 41:1313-1321, (2005).

“Antisense activity” means any detectable and/or measurable change in an amount of a target nucleic acid, or protein encoded by such target nucleic acid, attributable to the hybridization of an antisense compound to its target nucleic acid. In certain embodiments, antisense activity is a decrease in the amount of a target nucleic acid, or protein encoded by such target nucleic acid, compared to the amount of target nucleic acid, or protein encoded by such target nucleic acid, in the absence of the antisense compound. In certain embodiments, the change is detectable in a cell that has been contacted with the antisense compound or a cell lysate thereof. In certain embodiments, the change is detectable in a biological sample obtained from a subject to whom the the antisense compound has been administered. Non-limiting examples of biological samples include a liver biopsy sample, a blood sample, a plasma/serum sample, a saliva sample, and a urine sample.

“Antisense agent” means an antisense compound and optionally one or more additional features, such as a sense compound. An antisense agent includes, but is not limited to, an RNAi agent and an RNase H agent.

“Antisense compound” means an oligonucleotide, such as an antisense oligonucleotide, and optionally one or more additional features, such as a conjugate group

“Sense compound” means a sense oligonucleotide and optionally one or more additional features, such as a conjugate group.

“Antisense inhibition” means reduction of target nucleic acid levels in the presence of an antisense agent or antisense compound comprising an oligonucleotide complementary to a target nucleic acid, compared to target nucleic acid levels in the absence of the antisense compound.

“Antisense oligonucleotide” means an oligonucleotide, including the oligonucleotide portion of an antisense compound, that is capable of hybridizing to a target nucleic acid and is capable of at least one antisense activity. Antisense oligonucleotides include but are not limited to antisense RNAi oligonucleotides and antisense RNase H oligonucleotides.

“Sense oligonucleotide” means an oligonucleotide, including the oligonucleotide portion of a sense compound, that is capable of hybridizing to an antisense oligonucleotide.

“Bicyclic nucleoside” or “BNA” means a nucleoside comprising a bicyclic sugar moiety. “Bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the furanosyl sugar moiety is a ribosyl sugar moiety. In certain embodiments, the bicyclic sugar moiety does not comprise a furanosyl moiety.

“Branching group” means a group of atoms having at least 3 positions that are capable of forming covalent linkages to at least 3 groups. In certain embodiments, a branching group provides a plurality of reactive sites for connecting tethered ligands to an oligonucleotide via a conjugate linker and/or a cleavable moiety.

“Cell-targeting moiety” means a conjugate group or portion of a conjugate group that is capable of binding to a particular cell type or particular cell types.

“cEt” or “constrained ethyl” means a bicyclic furanosyl sugar moiety comprising a bridge connecting the 4′-carbon and the 2′-carbon, wherein the bridge has the formula: 4′—CH(CH₃)—O-2′. As used herein, “constrained ethyl nucleoside” or “cEt nucleoside” means:

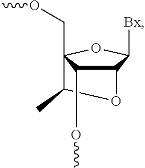

wherein Bx is a nucleobase

“Constrained ethyl” or “cEt” or “cEt sugar moiety” means the sugar moiety of a cEt nucleoside.

“Chemical modification” in a compound describes the substitutions or changes through chemical reaction, of any of the units in the compound. “Modified nucleoside” means a nucleoside having, independently, a modified sugar moiety and/or modified nucleobase. “Modified oligonucleotide” means an oligonucleotide comprising at least one modified internucleoside linkage, a modified sugar, and/or a modified nucleobase.

“Chemically distinct region” refers to a region of a compound that is in some way chemically different than another region of the same compound. For example, a region having 2′-O-methoxyethyl nucleotides is chemically distinct from a region having nucleotides without 2′-O-methoxyethyl modifications.

“Cleavable bond” means any chemical bond capable of being split. In certain embodiments, a cleavable bond is selected from among: an amide, a polyamide, an ester, an ether, one or both esters of a phosphodiester, a phosphate ester, a carbamate, a di-sulfide, or a peptide.

“Cleavable moiety” means a bond or group of atoms that is cleaved under physiological conditions, for example, inside a cell, an animal, or a human.

As used herein, “complementary” in reference to an oligonucleotide means that at least 70% of the nucleobases of the oligonucleotide and the nucleobases of another nucleic acid or one or more regions thereof are capable of hydrogen bonding with one another when the nucleobase sequence of the oligonucleotide and the other nucleic acid are aligned in opposing directions. “Complementary region” in reference to a region of an oligonucleotide means that at least 70% of the nucleobases of that region and the nucleobases of another nucleic acid or one or more regions thereof are capable of hydrogen bonding with one another when the nucleobase sequence of the oligonucleotide and the other nucleic acid are aligned in opposing directions. Complementary nucleobases mean nucleobases that are capable of forming hydrogen bonds with one another. Complementary nucleobase pairs include adenine (A) and thymine (T), adenine (A) and uracil (U), cytosine (C) and guanine (G), 5-methyl cytosine (mC) and guanine (G). Certain modified nucleobases that pair with natural nucleobases or with other modified nucleobases are known in the art and are not considered complementary nucleobases as defined herein unless indicated otherwise. For example, inosine can pair, but is not considered complementary, with adenosine, cytosine, or uracil. Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide. “Conjugate group” means a group of atoms that is attached to an oligonucleotide. Conjugate groups include a conjugate moiety and a conjugate linker that attaches the conjugate moiety to the oligonucleotide.

“Conjugate group” means a group of atoms that is attached to an oligonucleotide. Conjugate groups include a conjugate moiety and a conjugate linker that attaches the conjugate moiety to the oligonucleotide.

“Conjugate linker” means a group of atoms comprising at least one bond that connects a conjugate moiety to an oligonucleotide.

“Conjugate moiety” means a group of atoms that is attached to an oligonucleotide via a conjugate linker. A conjugate moiety modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Designing” or “Designed to” refer to the process of designing a compound that specifically hybridizes with a selected nucleic acid molecule.

“Diluent” means an ingredient in a composition that lacks pharmacological activity, but is pharmaceutically necessary or desirable. For example, the diluent in an injected composition can be a liquid, e.g. saline solution.

“Differently modified” mean chemical modifications or chemical substituents that are different from one another, including absence of modifications. Thus, for example, a 2′-MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe sugar moiety and an unmodified adenine nucleobase and a nucleoside comprising a 2′-OMe sugar moiety and an unmodified thymine nucleobase are not differently modified.

“Dose” means a specified quantity of a compound or pharmaceutical agent provided in a single administration, or in a specified time period. In certain embodiments, a dose may be administered in two or more boluses, tablets, or injections. For example, in certain embodiments, where subcutaneous administration is desired, the desired dose may require a volume not easily accommodated by a single injection. In such embodiments, two or more injections may be used to achieve the desired dose. In certain embodiments, a dose may be administered in two or more injections to minimize injection site reaction in a subject. In other embodiments, the COASY-specific inhibitor is administered by infusion over an extended period of time or continuously. Doses may be stated as the amount of COASY-specific inhibitor per hour, day, week or month.

“Double-stranded” in reference to an antisense agent means the antisense agent has two oligonucleotides that are sufficiently complementary to each other to form a duplex. “Double-stranded” in reference to a region or an oligonucleotide means a duplex formed by complementary strands of nucleic acids (including, but not limited to oligonucleotides) hybridized to one another. In certain embodiments, the two strands of a double-stranded region are separate molecules. In certain embodiments, the two strands are regions of the same molecule that has folded onto itself (e.g., a hairpin structure).

“COASY” means coenzyme A synthase and refers to any COASY nucleic acid or COASY protein. In certain embodiments, COASY includes a DNA sequence encoding COASY, an RNA sequence transcribed from DNA encoding COASY (including genomic DNA comprising introns and exons), or a COASY protein. The target may be referred to in either upper or lower case.

“COASY-specific inhibitor” refers to any agent capable of specifically reducing COASY RNA or COASY protein in a cell relative to a cell that is not exposed to the agent. COASY-specific inhibitors include nucleic acids (including antisense compounds), peptides, antibodies, small molecules, and other agents capable of inhibiting the expression or activity of COASY.

“Efficacy” means the ability to produce a desired effect.

“Expression” includes all the functions by which a gene's coded information is converted into structures present and operating in a cell. Such structures include, but are not limited to the products of transcription and translation.

“Gapmer” means a modified oligonucleotide comprising an internal region positioned between external regions having one or more nucleosides, wherein the nucleosides comprising the internal region are chemically distinct from the nucleoside or nucleosides comprising the external regions, and wherein the modified oligonucleotide supports RNAse H cleavage. The internal region may be referred to as the “gap” and the external regions may be referred to as the “wings.” In certain embodiments, the internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′-β-D-deoxynucleoside. In certain embodiments, the gap comprises one 2′-substituted nucleoside at position 1, 2, 3, 4, or 5 of the gap, and the remainder of the nucleosides of the gap are 2′-β-D-deoxynucleosides. As used herein, the term “MOE gapmer” indicates a gapmer having a gap comprising 2′-β-D-deoxynucleosides and wings comprising 2′-MOE nucleosides. As used herein, the term “mixed wing gapmer” indicates a gapmer having wings comprising modified nucleosides comprising at least two different sugar modifications. Unless otherwise indicated, a gapmer may comprise one or more modified internucleoside linkages and/or modified nucleobases and such modifications do not necessarily follow the gapmer pattern of the sugar modifications.

“Hybridization” means annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

“Immediately adjacent” means there are no intervening elements between the immediately adjacent elements of the same kind (e.g. no intervening nucleobases between the immediately adjacent nucleobases).

“Inhibiting the expression or activity” refers to a reduction or blockade of the expression or activity relative to the expression of activity in an untreated or control sample and does not necessarily indicate a total elimination of expression or activity.

“Internucleoside linkage” means a group or bond that forms a covalent linkage between adjacent nucleosides in an oligonucleotide. “Modified internucleoside linkage” means any internucleoside linkage other than a naturally occurring, phosphate internucleoside linkage. Non-phosphate linkages are referred to herein as modified internucleoside linkages.

“Linked nucleosides” means adjacent nucleosides linked together by an internucleoside linkage.

“Mismatch” or “non-complementary” means a nucleobase of a first oligonucleotide that is not complementary to the corresponding nucleobase of a second oligonucleotide or target nucleic acid when the first and second oligonucleotides are aligned. For example, nucleobases including but not limited to a universal nucleobase, inosine, and hypoxanthine, are capable of hybridizing with at least one nucleobase but are still mismatched or non-complementary with respect to nucleobase to which it hybridized. As another example, a nucleobase of a first oligonucleotide that is not capable of hybridizing to the corresponding nucleobase of a second oligonucleotide or target nucleic acid when the first and second oligonucleotides are aligned is a mismatch or non-complementary nucleobase.

“Modulating” refers to changing or adjusting a feature in a cell, tissue, organ or organism. For example, modulating COASY can mean to increase or decrease the level of COASY in a cell, tissue, organ or organism. A

“modulator” effects the change in the cell, tissue, organ or organism. For example, a compound can be a modulator of COASY that decreases the amount of COASY in a cell, tissue, organ or organism.

“Monomer” refers to a single unit of an oligomer. Monomers include, but are not limited to, nucleosides and nucleotides.

“Motif” means the pattern of unmodified and/or modified sugar moieties, nucleobases, and/or internucleoside linkages, in an oligonucleotide.

“Natural” or “naturally occurring” means found in nature.

“Non-bicyclic modified sugar” or “non-bicyclic modified sugar moiety” means a modified sugar moiety that comprises a modification, such as a substituent, that does not form a bridge between two atoms of the sugar to form a second ring.

“Nucleic acid” refers to molecules composed of monomeric nucleotides. A nucleic acid includes, but is not limited to, ribonucleic acids (RNA), deoxyribonucleic acids (DNA), single-stranded nucleic acids, and double-stranded nucleic acids.

“Nucleobase” means an unmodified nucleobase or a modified nucleobase. A nucleobase is a heterocyclic moiety. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one other nucleobase. A “5-methylcytosine” is a modified nucleobase. A universal base is a modified nucleobase that can pair with any one of the five unmodified nucleobases. “Nucleobase sequence” means the order of contiguous nucleobases in a nucleic acid or oligonucleotide independent of any sugar or internucleoside linkage.

“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

“Oligomeric agent” means an oligomeric compound and optionally one or more additional features, such as a second oligomeric compound. An oligomeric agent may be a single-stranded oligomeric compound or may be an oligomeric duplex formed by two complementary oligomeric compounds.

“Oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

“Oligomeric duplex” means a duplex formed by two oligomeric compounds having complementary nucleobase sequences.

“Oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications. “Parent oligonucleotide” means an oligonucleotide having a nucleobase sequence that is used as the basis of design for more oligonucleotides of similar sequence but with different lengths, motifs, and/or chemistries. The newly designed oligonucleotides may have the same or overlapping sequence as the parent oligonucleotide.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.

“Pharmaceutically acceptable carrier or diluent” means any substance suitable for use in administering to a subject. Certain such carriers enable pharmaceutical compositions to be formulated as, for example, tablets, pills, dragees, capsules, liquids, gels, syrups, slurries, suspension and lozenges for the oral ingestion by a subject. For example, a pharmaceutically acceptable carrier can be a sterile aqueous solution, sterile saline, sterile buffer solution such as PBS, or water-for-injection.

“Pharmaceutically acceptable salts” means physiologically and pharmaceutically acceptable salts of compounds, such as oligomeric compounds or oligonucleotides, i.e., salts that retain the desired biological activity of the parent compound and do not impart undesired toxicological effects thereto.

“Pharmaceutical composition” means a mixture of substances suitable for administering to a subject. For example, a pharmaceutical composition may comprise one or more compounds or salt thereof and a sterile aqueous solution.

“Phosphorothioate linkage” means a modified phosphate linkage in which one of the non-bridging oxygen atoms is replaced with a sulfur atom. A phosphorothioate internucleoside linkage is a modified internucleoside linkage.

“Phosphorus moiety” means a group of atoms comprising a phosphorus atom. In certain embodiments, a phosphorus moiety comprises a mono-, di-, or tri-phosphate, or phosphorothioate.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an oligomeric compound.

“Prodrug” means a compound in a form outside the body which, when administered to a subject, is metabolized to another form within the body or cells thereof. In certain embodiments, the metabolized form is the active, or more active, form of the compound (e.g., drug). Typically conversion of a prodrug within the body is facilitated by the action of an enzyme(s) (e.g., endogenous or viral enzyme) or chemical(s) present in cells or tissues, and/or by physiologic conditions.

“Reduce” means to bring down to a smaller extent, size, amount, or number. In certain embodiments, COASY (RNA or protein) is reduced in a cell or individual that is contacted or treated with a COASY-specific inhibitor, respectively, relative to a cell or individual that is not contacted or treated with a COASY-specific inhibitor, respectively.

“RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNAi (ssRNAi), and microRNA, including microRNA mimics. RNAi agents may comprise conjugate groups and/or terminal groups. In certain embodiments, an RNAi agent modulates the amount and/or activity, of a target nucleic acid. The term RNAi agent excludes antisense agents that act through RNase H.

“RNase H agent” means an antisense agent that acts through RNase H to modulate a target nucleic acid and/or protein encoded by a target nucleic acid. In certain embodiments, RNase H agents are single-stranded. In certain embodiments, RNase H agents are double-stranded. RNase H compounds may comprise conjugate groups and/or terminal groups. In certain embodiments, an RNase H agent modulates the amount and/or activity of a target nucleic acid. The term RNase H agent excludes antisense agents that act principally through RISC/Ago2.

“RefSeq No.” is a unique combination of letters and numbers assigned to a sequence to indicate the sequence is for a particular target transcript (e.g., target gene). Such sequence and information about the target gene (collectively, the gene record) can be found in a genetic sequence database. Genetic sequence databases include the NCBI Reference Sequence database, GenBank, the European Nucleotide Archive, and the DNA Data Bank of Japan (the latter three forming the International Nucleotide Sequence Database Collaboration or INSDC).

“Region” is defined as a portion of the target nucleic acid having at least one identifiable structure, function, or characteristic.

“Segments” are defined as smaller or sub-portions of regions within a nucleic acid.

“Single-stranded” in reference to an antisense agent means the antisense agent has only one oligonucleotide.

“Self-complementary” means an oligonucleotide that at least partially hybridizes to itself. A compound consisting of one oligonucleotide, wherein the oligonucleotide of the compound is self-complementary, is a single-stranded compound. A single-stranded compound may be capable of binding to a complementary compound to form a duplex.

“Sites,” are defined as unique nucleobase positions within a target nucleic acid.

“Specifically hybridizable” and “specific hybridization” refers to an oligonucleotide having a sufficient degree of complementarity between the oligonucleotide and a target nucleic acid to induce a desired effect, while exhibiting minimal or no effects on non-target nucleic acids. In certain embodiments, specific hybridization occurs under physiological conditions.

“Specifically inhibit” a target nucleic acid means to reduce or block expression of the target nucleic acid while exhibiting fewer, minimal, or no effects on non-target nucleic acids reduction and does not necessarily indicate a total elimination of the target nucleic acid's expression.

“Subject” means a human or non-human subject selected for treatment or therapy.

“Sugar moiety” means an unmodified sugar moiety or a modified sugar moiety. “Unmodified sugar moiety” or “unmodified sugar” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. “Modified sugar moiety” or “modified sugar” means a modified furanosyl sugar moiety or a sugar surrogate. “Modified furanosyl sugar moiety” means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars.

“Sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests.

“Target gene” refers to a gene encoding a target.

“Targeting” and “targeted” means specific hybridization of an antisense agent, antisense compound, or oligonucleotide to a target nucleic acid in order to induce a desired effect.

“Target nucleic acid,” “target RNA,” “target RNA transcript” and “nucleic acid target” all mean a nucleic acid capable of being targeted by compounds described herein. Target RNA means an RNA transcript and includes pre-mRNA and mature mRNA unless otherwise specified.

“Target region” means a portion of a target nucleic acid to which one or more compounds is targeted.

“Target segment” means the sequence of nucleotides of a target nucleic acid to which a compound described herein is targeted. “5′ target site” refers to the 5′-most nucleotide of a target segment. “3′ target site” refers to the 3′-most nucleotide of a target segment.

“Terminal group” means a chemical group or group of atoms that is covalently linked to a terminus of an oligonucleotide.

“Therapeutically effective amount” means an amount of a COASY-specific inhibitor or composition that provides a therapeutic benefit to a subject.

“Treat” refers to administering a compound or pharmaceutical composition to a subject in order to effect an alteration or improvement of a disease, disorder, or condition in the subject. In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

### Certain Embodiments

Certain embodiments provide COASY-specific inhibitors, compositions, and methods for treating a liver disease or disorder, or a symptom thereof, in a subject by administering the COASY-specific inhibitor or composition to the subject. Inhibition of COASY can lead to a decrease of COASY level or expression in order to treat a liver disease or disorder, or a symptom thereof. In certain embodiments, COASY-specific inhibitors are antisense agents, single-stranded antisense agents, double-stranded antisense agents, RNAi agents, RNase H agents, double-stranded siRNA, single-stranded RNAi (ssRNAi), microRNA, antisense compounds, oligonucleotides, peptides, antibodies, small molecules, and other agents capable of inhibiting the expression or activity of COASY. In certain embodiments, the subject is human. In certain embodiments, the antisense agent or RNAi agent comprises ribonucleotides and is double-stranded. In certain embodiments, the antisense agent, single-stranded antisense agent, double-stranded antisense agent, RNAi agent, RNase H agent, double-stranded siRNA, single-stranded RNAi (ssRNAi), microRNA, or antisense compound comprises a modified oligonucleotide.

In any of the foregoing embodiments, the modified oligonucleotide consisting of 8 to 80, 10 to 30, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked nucleosides.

In certain embodiments, at least one internucleoside linkage of said modified oligonucleotide is a modified internucleoside linkage. In certain embodiments, at least one internucleoside linkage is a phosphorothioate internucleoside linkage. In certain embodiments, the internucleoside linkages are phosphorothioate linkages and phosphate ester linkages.

In certain embodiments, any of the foregoing oligonucleotides comprises at least one modified sugar. In certain embodiments, at least one modified sugar comprises a 2′-O-methoxyethyl group. In certain embodiments, at least one modified sugar is a bicyclic sugar, such as a 4′—CH(CH₃)—O-2′ group, a 4′—CH₂—O-2′ group, or a 4′—(CH₂)₂—O-2′group. In certain embodiments, at least one modified sugar comprises a 2′—F group or a 2′-OMe group.

In certain embodiments, at least one nucleoside of said modified oligonucleotide comprises a modified nucleobase. In certain embodiments, the modified nucleobase is a 5-methylcytosine.

In certain embodiments, a COASY-specific inhibitor or composition comprises a modified oligonucleotide comprising: a) a gap segment consisting of linked 2′-deoxynucleosides; b) a 5′ wing segment consisting of linked nucleosides; and c) a 3′ wing segment consisting of linked nucleosides. The gap segment is positioned between the 5′ wing segment and the 3′ wing segment and each nucleoside of each wing segment comprises a modified sugar. In certain embodiments, at least one internucleoside linkage is a phosphorothioate linkage. In certain embodiments, and at least one cytosine is a 5-methylcytosine.

In certain embodiments, the compounds or compositions disclosed herein further comprise a pharmaceutically acceptable carrier or diluent.

In certain embodiments, the COASY-specific inhibitor or composition is co-administered with a second agent.

In certain embodiments, the COASY-specific inhibitor or composition and the second agent are administered concomitantly.

In certain embodiments, COASY-specific inhibitors can be used in methods of inhibiting expression of COASY in a cell. In certain embodiments, COASY-specific inhibitors can be used in methods of treating a liver disease or disorder including, but not limited to, fatty liver disease, chronic liver disease, liver cirrhosis, hepatic steatosis, steatohepatitis, NAFLD, ASH, or NASH.

In certain embodiments, COASY antisense agents can be used in methods of reducing expression of COASY in a cell. In certain embodiments, COASY specific antisense agents can be used in methods of treating a liver disease, metabolic disease, or cardiovascular disease or disorder including, but not limited to, metabolic syndrome, liver disease, fatty liver disease, chronic liver disease, liver cirrhosis, hepatic steatosis, steatohepatitis, NAFLD, ASH, and NASH.

### Certain Indications

Certain embodiments provided herein relate to methods of inhibiting COASY expression or activity, which can be useful for treating a disease associated with COASY in a subject, such as NASH, by administration of a COASY-specific inhibitor.

In certain embodiments, a method of inhibiting expression or activity of COASY in a cell comprises contacting the cell with a COASY-specific inhibitor, thereby inhibiting expression or activity of COASY in the cell. In certain embodiments, the cell is a liver cell. In certain embodiments, the cell is in the liver. In certain embodiments, the cell is in the liver of a subject who has a disease, disorder, condition, symptom, or physiological marker associated with a liver disease or disorder. In certain embodiments, the liver disease or disorder is fatty liver disease, chronic liver disease, liver cirrhosis, hepatic steatosis, steatohepatitis, NAFLD, ASH, or NASH. In certain embodiments, the disease is NASH. In certain embodiments, the COASY-specific inhibitor is an antisense agent, single-stranded antisense agent, double-stranded antisense agent, RNAi agent, RNase H agent, double-stranded siRNA, single-stranded RNAi (ssRNAi), microRNA, antisense compound, peptide, antibody, small molecule or other agent capable of inhibiting the expression or activity of the COASY. In certain embodiments, the antisense agent, single-stranded antisense agent, double-stranded antisense agent, RNAi agent, RNase H agent, double-stranded siRNA, single-stranded RNAi (ssRNAi), microRNA, or antisense compound comprises a modified oligonucleotide consisting of 8 to 80 linked nucleosides. In certain embodiments, the modified oligonucleotide consists of 10 to 30 linked nucleosides. In certain embodiments, the antisense agent comprising a modified oligonucleotide can be single-stranded. In certain embodiments, the antisense agent comprising a modified oligonucleotide can be double-stranded. In certain embodiments, the double-stranded antisense agent comprises an antisense compound and a sense compound. In certain embodiments, the antisense agent is a double-stranded siRNA targeted to COASY.

In certain embodiments, a method of treating one or more diseases, disorders, conditions, symptoms or physiological markers associated with COASY comprises administering to the subject a COASY-specific inhibitor. In certain embodiments, a method of treating a disease, disorder, condition, symptom, or physiological marker associated with a liver disease or disorder in a subject comprises administering to the subject a COASY-specific inhibitor, thereby treating the disease. In certain embodiments, the subject is identified as having the disease, disorder, condition, symptom or physiological marker. In certain embodiments, the liver disease or disorder is fatty liver disease, chronic liver disease, liver cirrhosis, hepatic steatosis, steatohepatitis, NAFLD, ASH, or NASH. In certain embodiments, the disease is NASH. In certain embodiments, the COASY-specific inhibitor is administered to the subject parenterally. In certain embodiments, the parenteral administration is subcutaneous administration. In certain embodiments, the subject is human. In certain embodiments, the COASY-specific inhibitor is an antisense agent, single-stranded antisense agent, double-stranded antisense agent, RNAi agent, RNase H agent, double-stranded siRNA, single-stranded RNAi (ssRNAi), microRNA, antisense compound, peptide, antibody, small molecule or other agent capable of inhibiting the expression or activity of the COASY. In certain embodiments, the antisense agent, single-stranded antisense agent, double-stranded antisense agent, RNAi agent, RNase H agent, double-stranded siRNA, single-stranded RNAi (ssRNAi), microRNA, or antisense compound comprises a modified oligonucleotide consisting of 8 to 80 linked nucleosides. In certain embodiments, the modified oligonucleotide consists of 10 to 30 linked nucleosides. In certain embodiments, the antisense agent comprising a modified oligonucleotide can be single-stranded. In certain embodiments, the antisense agent comprising a modified oligonucleotide can be double-stranded. In certain embodiments, the double-stranded antisense agent comprises an antisense compound and a sense compound. In certain embodiments, the antisense agent is a double-stranded siRNA targeted to COASY.

In certain embodiments, a method of reducing hepatic steatosis, liver fibrosis, triglyceride synthesis, plasma lipid levels, hepatic lipids, ALT levels, NAFLD Activity Score (NAS), or plasma cholesterol levels, or a combination thereof, in a subject comprises administering to the subject a COASY-specific inhibitor. In certain embodiments, hepatic steatosis, liver fibrosis, triglyceride synthesis, plasma lipid levels, hepatic lipids, ALT levels, NAFLD Activity Score (NAS), or plasma cholesterol levels, or a combination thereof, is reduced in a subject that is administered a COASY-specific inhibitor, relative to hepatic steatosis, liver fibrosis, triglyceride synthesis, plasma lipid levels, hepatic lipids, ALT levels, NAFLD Activity Score (NAS), or plasma cholesterol levels, or a combination thereof in the subject before administration. In certain embodiments, hepatic steatosis, liver fibrosis, triglyceride synthesis, plasma lipid levels, hepatic lipids, ALT levels, NAFLD Activity Score (NAS), or plasma cholesterol levels, or a combination thereof, is reduced in a subject that is administered a COASY-specific inhibitor, relative to hepatic steatosis, liver fibrosis, triglyceride synthesis, plasma lipid levels, hepatic lipids, ALT levels, NAFLD Activity Score (NAS), or plasma cholesterol levels, or a combination thereof in a control subject that does not receive the COASY-specific inhibitor. In certain embodiments, the subject is identified as having a disease, disorder, condition, symptom, or physiological marker associated with a liver disease or disorder. In certain embodiments, the liver disease or disorder is fatty liver disease, chronic liver disease, liver cirrhosis, hepatic steatosis, steatohepatitis, NAFLD, ASH, or NASH. In certain embodiments, the disease is NASH. In certain embodiments, the COASY-specific inhibitor is administered to the subject parenterally. In certain embodiments, the parenteral administration is subcutaneous administration. In certain embodiments, the subject is human. In certain embodiments, the COASY-specific inhibitor is an antisense agent, single-stranded antisense agent, double-stranded antisense agent, RNAi agent, RNase H agent, double-stranded siRNA, single-stranded RNAi (ssRNAi), microRNA, antisense compound, peptide, antibody, small molecule or other agent capable of inhibiting the expression or activity of the COASY. In certain embodiments, the antisense agent, single-stranded antisense agent, double-stranded antisense agent, RNAi agent, RNase H agent, double-stranded siRNA, single-stranded RNAi (ssRNAi), microRNA, or antisense compound comprises a modified oligonucleotide consisting of 8 to 80 linked nucleosides. In certain embodiments, the modified oligonucleotide consists of 10 to 30 linked nucleosides. In certain embodiments, the antisense agent comprising a modified oligonucleotide can be single-stranded. In certain embodiments, the antisense agent comprising a modified oligonucleotide can be double-stranded. In certain embodiments, the double-stranded antisense agent comprises an antisense compound and a sense compound. In certain embodiments, the antisense agent is a double-stranded siRNA targeted to COASY.

Certain embodiments are drawn to compounds and compositions described herein for use in therapy. Certain embodiments are drawn to a COASY-specific inhibitor or composition comprising a COASY-specific inhibitor for use in treating one or more diseases, disorders, conditions, symptoms or physiological markers associated with COASY. Certain embodiments are drawn to a COASY-specific inhibitor or composition for use in treating a liver disease or disorder, or a symptom or physiological marker thereof. In certain embodiments, the liver disease or disorder is fatty liver disease, chronic liver disease, liver cirrhosis, hepatic steatosis, steatohepatitis, NAFLD, ASH, or NASH. In certain embodiments, the disease is a liver disease or disorder. In certain embodiments, the COASY-specific inhibitor is an antisense agent, single-stranded antisense agent, double-stranded antisense agent, RNAi agent, RNase H agent, double-stranded siRNA, single-stranded RNAi (ssRNAi), microRNA, antisense compound, peptide, antibody, small molecule or other agent capable of inhibiting the expression or activity of the COASY. In certain embodiments, the antisense agent, single-stranded antisense agent, double-stranded antisense agent, RNAi agent, RNase H agent, double-stranded siRNA, single-stranded RNAi (ssRNAi), microRNA, or antisense compound comprises a modified oligonucleotide consisting of 8 to 80 linked nucleosides. In certain embodiments, the modified oligonucleotide consists of 10 to 30 linked nucleosides. In certain embodiments, the antisense agent comprising a modified oligonucleotide can be single-stranded. In certain embodiments, the antisense agent comprising a modified oligonucleotide can be double-stranded. In certain embodiments, the double-stranded antisense agent comprises an antisense compound and a sense compound. In certain embodiments, the antisense agent is a double-stranded siRNA targeted to COASY.

Certain embodiments are drawn to a COASY-specific inhibitor or composition comprising a COASY-specific inhibitor for use in reducing hepatic steatosis, liver fibrosis, plasma lipid levels, plasma triglyceride levels, plasma cholesterol levels, ALT levels, NAFLD Activity Score (NAS), hepatic lipidlevels, hepatic triglyceride levels, or hepatic cholesterol levels, or a combination thereof, in a subject. In certain embodiments, hepatic steatosis, liver fibrosis, plasma lipid levels, plasma triglyceride levels, plasma cholesterol levels, ALT levels, NAFLD Activity Score (NAS), hepatic lipid levels, hepatic triglyceride levels, or hepatic cholesterol levels, or a combination thereof, is reduced in a subject that is administered a COASY-specific inhibitor, relative to hepatic steatosis, liver fibrosis, plasma lipid levels, plasma triglyceride levels, plasma cholesterol levels, ALT levels, NAFLD Activity Score (NAS), hepatic lipid levels, hepatic triglyceride levels, or hepatic cholesterol levels, or a combination thereof in the subject before administration. In certain embodiments, hepatic steatosis, liver fibrosis, plasma lipid levels, plasma triglyceride levels, plasma cholesterol levels, ALT levels, NAFLD Activity Score (NAS), hepatic lipid levels, hepatic triglyceride levels, or hepatic cholesterol levels, or a combination thereof, is reduced in a subject that is administered a COASY-specific inhibitor, relative to hepatic steatosis, liver fibrosis, plasma lipid levels, plasma triglyceride levels, plasma cholesterol levels, ALT levels, NAFLD Activity Score (NAS), hepatic lipid levels, hepatic triglyceride levels, or hepatic cholesterol levels, or a combination thereofin a control subject that does not receive the COASY-specific inhibitor. In certain embodiments, the COASY-specific inhibitor or composition is provided for use in reducing hepatic steatosis in the subject. In certain embodiments, the COASY-specific inhibitor or composition is provided for use in reducing liver fibrosis in the subject. In certain embodiments, the COASY-specific inhibitor or composition is provided for use in reducing plasma triglyceride levels in the subject. In certain embodiments, the COASY-specific inhibitor or composition is provided for use in reducing hepatic triglyceride levels in the subject. In certain embodiments, the COASY-specific inhibitor or composition is provided for use in reducing plasma lipid levels in the subject. In certain embodiments, the COASY-specific inhibitor or composition is provided for use in reducing hepatic lipids in the subject. In certain embodiments, the COASY-specific inhibitor or composition is provided for use in reducing ALT levels in the subject. In certain embodiments, the COASY-specific inhibitor or composition is provided for use in reducing NAFLD Activity Score (NAS) in the subject. In certain embodiments, the COASY-specific inhibitor or composition is provided for use in reducing plasma cholesterol levels in the subject. In certain embodiments, the subject is identified as having a disease, disorder, condition, symptom, or physiological marker associated with a liver disease or disorder. In certain embodiments, the liver disease or disorder is fatty liver disease, chronic liver disease, liver cirrhosis, hepatic steatosis, steatohepatitis, NAFLD, ASH, or NASH. In certain embodiments, the disease is NASH. In certain embodiments, the subject is a human subject. In certain embodiments, the COASY-specific inhibitor is an antisense agent, single-stranded antisense agent, double-stranded antisense agent, RNAi agent, RNase H agent, double-stranded siRNA, single-stranded RNAi (ssRNAi), microRNA, antisense compound, peptide, antibody, small molecule or other agent capable of inhibiting the expression or activity of the COASY. In certain embodiments, the antisense agent, single-stranded antisense agent, double-stranded antisense agent, RNAi agent, RNase H agent, double-stranded siRNA, single-stranded RNAi (ssRNAi), microRNA, or antisense compound comprises a modified oligonucleotide consisting of 8 to 80 linked nucleosides. In certain embodiments, the modified oligonucleotide consists of 10 to 30 linked nucleosides. In certain embodiments, the antisense agent comprising a modified oligonucleotide can be single-stranded. In certain embodiments, the antisense agent comprising a modified oligonucleotide can be double-stranded. In certain embodiments, the double-stranded antisense agent comprises an antisense compound and a sense compound. In certain embodiments, the antisense agent is a double-stranded siRNA targeted to COASY.

Certain embodiments are drawn to use of COASY-specific inhibitors or compositions described herein for the manufacture or preparation of a medicament for therapy. Certain embodiments are drawn to the use of a COASY-specific inhibitor or composition as described herein in the manufacture or preparation of a medicament for treating one or more diseases, disorders, conditions, symptoms or physiological markers associated with COASY. In certain embodiments, the COASY-specific inhibitor or composition as described herein is used in the manufacture or preparation of a medicament for treating a liver disease or disorder, or a symptom or physiological marker thereof. In certain embodiments, the liver disease or disorder is fatty liver disease, chronic liver disease, liver cirrhosis, hepatic steatosis, steatohepatitis, NAFLD, ASH, or NASH. In certain embodiments, the disease is NASH. In certain embodiments, the COASY-specific inhibitor is an antisense agent, single-stranded antisense agent, double-stranded antisense agent, RNAi agent, RNase H agent, double-stranded siRNA, single-stranded RNAi (ssRNAi), microRNA, antisense compound, peptide, antibody, small molecule or other agent capable of inhibiting the expression or activity of the COASY. In certain embodiments, the antisense agent, single-stranded antisense agent, double-stranded antisense agent, RNAi agent, RNase H agent, double-stranded siRNA, single-stranded RNAi (ssRNAi), microRNA, or antisense compound comprises a modified oligonucleotide consisting of 8 to 80 linked nucleosides. In certain embodiments, the modified oligonucleotide consists of 10 to 30 linked nucleosides. In certain embodiments, the antisense agent comprising a modified oligonucleotide can be single-stranded. In certain embodiments, the antisense agent comprising a modified oligonucleotide can be double-stranded. In certain embodiments, the double-stranded antisense agent comprises an antisense compound and a sense compound. In certain embodiments, the antisense agent is a double-stranded siRNA targeted to COASY.

Certain embodiments are drawn to the use of a COASY-specific inhibitor or composition for the manufacture or preparation of a medicament for reducing hepatic steatosis, liver fibrosis, triglyceride synthesis, plasma lipid levels, hepatic lipids, ALT levels, NAFLD Activity Score (NAS), or plasma cholesterol levels, or a combination thereof, in a subject having a liver disease or disorder. Certain embodiments are drawn to use of a COASY-specific inhibitor or composition in the manufacture or preparation of a medicament for reducing hepatic steatosis in the subject. Certain embodiments are drawn to use of a COASY-specific inhibitor in the manufacture or preparation of a medicament for reducing liver fibrosis in the subject. Certain embodiments are drawn to use of a COASY-specific inhibitor in the manufacture or preparation of a medicament for reducing triglyceride synthesis in the subject. Certain embodiments are drawn to use of a COASY-specific inhibitor in the manufacture or preparation of a medicament for reducing plasma lipid levels in the subject. Certain embodiments are drawn to use of a COASY-specific inhibitor in the manufacture or preparation of a medicament for reducing hepatic lipids in the subject. Certain embodiments are drawn to use of a COASY-specific inhibitor in the manufacture or preparation of a medicament for reducing ALT levels in the subject. Certain embodiments are drawn to use of a COASY-specific inhibitor in the manufacture or preparation of a medicament for reducing NAFLD Activity Score (NAS) in the subject. Certain embodiments are drawn to use of a COASY-specific inhibitor or composition in the manufacture or preparation of a medicament for reducing plasma cholesterol levels in the subject. In certain embodiments, the COASY-specific inhibitor or composition comprises an antisense agent, single-stranded antisense agent, double-stranded antisense agent, RNAi agent, RNase H agent, double-stranded siRNA, single-stranded RNAi (ssRNAi), microRNA, antisense compound, peptide, antibody, small molecule or other agent capable of inhibiting the expression or activity of the COASY. In certain embodiments, the antisense agent, single-stranded antisense agent, double-stranded antisense agent, RNAi agent, RNase H agent, double-stranded siRNA, single-stranded RNAi (ssRNAi), microRNA, or antisense compound comprises a modified oligonucleotide consisting of 8 to 80 linked nucleosides. In certain embodiments, the modified oligonucleotide consists of 10 to 30 linked nucleosides. In certain embodiments, the antisense agent comprising a modified oligonucleotide can be single-stranded. In certain embodiments, the antisense agent comprising a modified oligonucleotide can be double-stranded. In certain embodiments, the double-stranded antisense agent comprises an antisense compound and a sense compound. In certain embodiments, the antisense agent is a double-stranded siRNA targeted to COASY.

In any of the foregoing methods or uses, the antisense agent can comprise an antisense compound targeted to COASY. In certain embodiments, the antisense compound comprises an oligonucleotide, for example an oligonucleotide consisting of 8 to 80 linked nucleosides, 10 to 30 linked nucleosides, 12 to 30 linked nucleosides, or 20 linked nucleosides. In certain embodiments, the oligonucleotide comprises at least one modified internucleoside linkage, at least one modified sugar and/or at least one modified nucleobase. In certain embodiments, the modified internucleoside linkage is a phosphorothioate internucleoside linkage, the modified sugar is abicyclic sugar or a 2′-O-methoxyethyl, and the modified nucleobase is a 5-methylcytosine. In certain embodiments, the modified oligonucleotide comprises a gap segment consisting of linked 2′-deoxynucleosides; a 5′ wing segment consisting of linked nucleosides; and a 3′ wing segment consisting of linked nucleosides, wherein the gap segment is positioned immediately adjacent to and between the 5′ wing segment and the 3′ wing segment and wherein each nucleoside of each wing segment comprises a modified sugar. In certain embodiments, the antisense agent is single-stranded. In certain embodiments, the antisense agent is double-stranded. In certain embodiments, the modified oligonucleotide consists of 12 to 30 linked nucleosides. In certain embodiments, compositions disclosed herein comprise an antisense agent described herein and a pharmaceutically acceptable carrier or diluent.

In any of the foregoing methods or uses, the COASY-specific inhibitor or composition comprises or consists of a modified oligonucleotide 12 to 30 linked nucleosides in length, wherein the modified oligonucleotide comprises:
a gap segment consisting of linked 2′-deoxynucleosides;
a 5′ wing segment consisting of linked nucleosides; and
a 3′ wing segment consisting of linked nucleosides;

wherein the gap segment is positioned between the 5′ wing segment and the 3′ wing segment and wherein each nucleoside of each wing segment comprises a modified sugar.

In any of the foregoing methods or uses, the COASY-specific inhibitor or composition can be administered parenterally. For example, in certain embodiments the COASY-specific inhibitor or composition can be administered through injection or infusion. Parenteral administration includes subcutaneous administration, intravenous administration, intramuscular administration, intraarterial administration, intraperitoneal administration, or intracranial administration. In certain embodiments, the parenteral administration is subcutaneous administration. In certain embodiments, the COASY-specific inhibitor or composition is co-administered with a second agent. In certain embodiments, the COASY-specific inhibitor or composition and the second agent are administered concomitantly.

### Certain Compounds

In certain embodiments, antisense agents described herein comprise antisense compounds. In certain embodiments, the antisense compound comprises a modified oligonucleotide. In certain embodiments, the modified oligonucleotide has a nucleobase sequence complementary to that of a target nucleic acid.

In certain embodiments, an antisense agent described herein comprises or consists of a modified oligonucleotide. In certain embodiments, the modified oligonucleotide has a nucleobase sequence complementary to that of a target nucleic acid.

In certain embodiments, an antisense agent is single-stranded. In certain embodiments, a single-stranded antisense agent comprises or consists of an antisense compound. In certain embodiments, such an antisense compound comprises or consists of an oligonucleotide. In certain embodiments, the oligonucleotide is an antisense oligonucleotide.

In certain embodiments, the oligonucleotide is modified. In certain embodiments, the oligonucleotide of a single-stranded antisense agent or antisense compound comprises a self-complementary nucleobase sequence. In certain embodiments, a single-stranded antisense agent comprises an antisense compound, which comprises a modified oligonucleotide and a conjugate group.

In certain embodiments, antisense agents are double-stranded. In certain embodiments, double-stranded antisense agents comprise a first modified oligonucleotide having a region complementary to a target nucleic acid and a second modified oligonucleotide having a region complementary to the first modified oligonucleotide. In certain embodiments, the modified oligonucleotide is an RNA oligonucleotide. In such embodiments, the thymine nucleobase in the modified oligonucleotide is replaced by a uracil nucleobase. In certain embodiments, a double-stranded antisense agent comprises a conjugate group. In certain embodiments, a double-stranded antisense agent comprises an antisense compound and a sense compound, wherein the sense compound comprises a conjugate group. In certain embodiments, each modified oligonucleotide is 12-30 linked nucleosides in length.

Examples of single-stranded and double-stranded antisense agents include but are not limited to oligonucleotides, siRNAs, microRNA targeting oligonucleotides, and single-stranded RNAi compounds, such as small hairpin RNAs (shRNAs), single-stranded siRNAs (ssRNAs), and microRNA mimics.

In certain embodiments, an antisense agent described herein comprises an oligonucleotide having a nucleobase sequence that, when written in the 5′ to 3′ direction, comprises the reverse complement of the target segment of a target nucleic acid to which it is targeted.

In certain embodiments, an antisense agent, antisense compound, or sense compound described herein comprises an oligonucleotide consisting of 10 to 30 linked nucleosides. In certain embodiments, the oligonucleotide consists of 12 to 30 linked nucleosides. In certain embodiments, the oligonucleotide consists of 12 to 22 linked nucleosides.

In certain embodiments, the oligonucleotide consists of 14 to 30 linked nucleosides. In certain embodiments, the oligonucleotide consists of 14 to 20 linked nucleosides. In certain embodiments, the oligonucleotide consists of 15 to 30 linked linked nucleosides. In certain embodiments, the oligonucleotide consists of 15 to 20 linked nucleosides. In certain embodiments, the oligonucleotide consists of 16 to 30 linked nucleosides. In certain embodiments, the oligonucleotide consists of 16 to 20 linked nucleosides. In certain embodiments, the oligonucleotide consists of 17 to 30 linked nucleosides.

In certain embodiments, the oligonucleotide consists of 17 to 20 linked nucleosides. In certain embodiments, the oligonucleotide consists of 18 to 30 linked nucleosides. In certain embodiments, the oligonucleotide consists of 18 to 21 linked nucleosides. In certain embodiments, the oligonucleotide consists of 18 to 20 linked nucleosides. In certain embodiments, the oligonucleotide consists of 20 to 30 linked nucleosides. In certain embodiments, oligonucleotides consist of 12 to 30 linked nucleosides, 14 to 30 linked nucleosides, 14 to 20 linked nucleosides, 15 to 30 linked nucleosides, 15 to 20 linked nucleosides, 16 to 30 linked nucleosides, 16 to 20 linked nucleosides, 17 to 30 linked nucleosides, 17 to 20 linked nucleosides, 18 to 30 linked nucleosides, 18 to 20 linked nucleosides, 18 to 21 linked nucleosides, 20 to 30 linked nucleosides, or 12 to 22 linked nucleosides. In certain embodiments, an oligonucleotide consists of 14 linked nucleosides. In certain embodiments, an oligonucleotide consists of 16 linked nucleosides. In certain embodiments, an oligonucleotide consists of 17 linked nucleosides. In certain embodiments, an oligonucleotide consists of 18 linked nucleosides. In certain embodiments, an oligonucleotide consists of 19 linked nucleosides. In certain embodiments, an oligonucleotide consists of 20 linked nucleosides. In other embodiments, an oligonucleotide consists of 8 to 80, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked nucleosides. In certain such embodiments, an oligonucleotide consists of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked nucleosides, or a range defined by any two of the above values. In certain embodiments, the oligonucleotide is a modified oligonucleotide. In certain embodiments, the oligonucleotide is an antisense oligonucleotide. In certain embodiments, the oligonucleotide is a sense oligonucleotide.

In certain embodiments, antisense agents described herein are interfering RNA compounds (RNAi), which include double-stranded RNA compounds (also referred to as short-interfering RNA or siRNA) and single-stranded RNAi compounds (or ssRNAi). Such compounds work at least in part through the RISC pathway to degrade and/or sequester a target nucleic acid (thus, include microRNA/microRNA-mimic compounds). As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics.

In certain embodiments, a double-stranded antisense agent comprises a first oligonucleotide comprising the nucleobase sequence complementary to a target region of a COASY nucleic acid and a second oligonucleotide. In certain embodiments, the double-stranded compound comprises ribonucleotides in which the first oligonucleotide has uracil (U) in place of thymine (T) and is complementary to a target region. In certain embodiments, a double-stranded compound comprises (i) a first oligonucleotide comprising a nucleobase sequence complementary to a target region of a COASY nucleic acid, and (ii) a second oligonucleotide. In certain embodiments, the double-stranded antisense agent comprises one or more modified nucleotides in which the 2′ position in the sugar contains a halogen (such as fluorine group; 2′-F) or contains an alkoxy group (such as a methoxy group; 2′-OMe). In certain embodiments, the double-stranded antisense agent comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the dsRNA compound. In certain embodiments, the double-stranded antisense agent comprises one or more linkages between adjacent nucleotides other than a naturally-occurring phosphodiester linkage. Examples of such linkages include phosphoramide, phosphorothioate, and phosphorodithioate linkages. The double-stranded compounds may also be chemically modified nucleic acid molecules as taught in U.S. Pat. No. 6,673,661. In other embodiments, the dsRNA contains one or two capped strands, as disclosed, for example, by WO 00/63364, filed Apr. 19, 2000. In certain embodiments, the first oligonucleotide of the double-stranded antisense agent is an siRNA guide strand and the second oligonucleotide of the double-stranded compound is an siRNA passenger strand. In certain embodiments, the second oligonucleotide of the double-stranded antisense agent is complementary to the first oligonucleotide. In certain embodiments, the first oligonucleotide of the double-stranded antisense agent consists of 16, 17, 18, 19, 20, 21, 22, or 23 linked nucleosides. In certain embodiments, the second oligonucleotide of the double-stranded antisense agent consists of 16, 17, 18, 19, 20, 21, 22, or 23 linked nucleosides.

In certain embodiments, a single-stranded antisense agent described herein can comprise any of the oligonucleotide sequences targeted to COASY described herein. In certain embodiments, such a single-stranded antisense agent is a single-stranded RNAi (ssRNAi) agent. In certain embodiments, a ssRNAi agent comprises the nucleobase sequence complementary to a target region of a COASY nucleic acid. In certain embodiments, the ssRNAi agent comprises ribonucleotides in which uracil (U) is in place of thymine (T). In certain embodiments, ssRNAi agent comprises a nucleobase sequence complementary to a target region of a COASY nucleic acid. In certain embodiments, a ssRNAi agent comprises one or more modified nucleotides in which the 2′ position in the sugar contains a halogen (such as fluorine group; 2′-F) or contains an alkoxy group (such as a methoxy group; 2′-OMe). In certain embodiments, a ssRNAi agent comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the ssRNAi agent. In certain embodiments, the ssRNAi agent comprises one or more linkages between adjacent nucleotides other than a naturally-occurring phosphodiester linkage. Examples of such linkages include phosphoramide, phosphorothioate, and phosphorodithioate linkages. The ssRNAi agents may also be chemically modified nucleic acid molecules as taught in U.S. Pat. No. 6,673,661. In other embodiments, the ssRNAi agent contains a capped strand, as disclosed, for example, by WO 00/63364, filed Apr. 19, 2000. In certain embodiments, the ssRNAi agent consists of 16, 17, 18, 19, 20, 21, 22, or 23 linked nucleosides.

In certain embodiments, antisense agents described herein comprise modified oligonucleotides. Certain modified oligonucleotides have one or more asymmetric center and thus give rise to enantiomers, diastereomers, and other stereoisomeric configurations that may be defined, in terms of absolute stereochemistry, as (R) or (S), as a or $ such as for sugar anomers, or as (D) or (L) such as for amino acids etc. Included in the modified oligonucleotides provided herein are all such possible isomers, including their racemic and optically pure forms, unless specified otherwise. Likewise, all cis- and trans-isomers and tautomeric forms are also included.

### Certain Mechanisms

In certain embodiments, antisense agents described herein selectively affect one or more target nucleic acid. Such selective antisense agents comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of an antisense agent described herein to a target nucleic acid results in recruitment of a protein that cleaves the target nucleic acid. For example, certain antisense agents described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, antisense agents described herein are sufficiently “DNA-like” to elicit RNase H activity. Further, in certain embodiments, one or more non-DNA-like nucleoside in the gap of a gapmer is tolerated.

In certain antisense activities, antisense agents described herein or a portion of the antisense agent is loaded into an RNA-induced silencing complex (RISC), ultimately resulting in cleavage of the target nucleic acid. For example, certain antisense agents described herein result in cleavage of the target nucleic acid by Argonaute. In certain embodiments, antisense agents that are loaded into RISC are RNAi agents. RNAi agents may be double-stranded (siRNA) or single-stranded (ssRNAi).

In certain embodiments, hybridization of antisense agents described herein to a target nucleic acid does not result in recruitment of a protein that cleaves that target nucleic acid. In certain such embodiments, hybridization of the antisense agents to the target nucleic acid results in alteration of splicing of the target nucleic acid. In certain embodiments, hybridization of the antisense agents to a target nucleic acid results in inhibition of a binding interaction between the target nucleic acid and a protein or other nucleic acid. In certain such embodiments, hybridization of the antisense agents to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or individual.

### Target Nucleic Acids, Target Regions and Nucleotide Sequences

In certain embodiments, antisense agents described herein comprise or consist of an oligonucleotide comprising a region that is complementary to a COASY nucleic acid.

Nucleotide sequences that encode COASY include, without limitation, the following RefSEQ Nos.: ENSEMBL Accession No. ENSMUSG00000001755.12 from version 102: November 2020 (incorporated by reference, disclosed herein as SEQ ID NO: 1); GENBANK Accession No. NM_001305982.1 (incorporated by reference, disclosed herein as SEQ ID NO: 2); GENBANK Accession No. NM_025233.7 (incorporated by reference, disclosed herein as SEQ ID NO: 3); and GENBANK Accession No. NG_034110.1 (incorporated by reference, disclosed herein as SEQ ID NO: 4).

### Hybridization

In some embodiments, hybridization occurs between an antisense agent disclosed herein and a COASY nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Hybridization conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense agents provided herein are specifically hybridizable with a COASY nucleic acid.

### Complementarity

An oligonucleotide is said to be complementary to another nucleic acid when the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to adenine (A) and thymine (T), adenine (A) and uracil (U), cytosine (C) and guanine (G), and 5-methylcytosine (mC) and guanine (G) unless otherwise specified. Complementary oligonucleotides and/or nucleic acids need not have nucleobase complementarity at each nucleoside and may include one or more nucleobase mismatches. An oligonucleotide is fully complementary or 100% complementary when such oligonucleotides have nucleobase matches at each nucleoside without any nucleobase mismatches.

In certain embodiments, antisense agents described herein comprise or consist of modified oligonucleotides. In certain embodiments, antisense agents described herein are antisense compounds. Non-complementary nucleobases between an oligonucleotide and a COASY nucleic acid may be tolerated provided that the oligonucleotide remains able to specifically hybridize to a target nucleic acid. Moreover, an oligonucleotide may hybridize over one or more segments of a COASY nucleic acid such that intervening or adjacent segments are not involved in the hybridization event (e.g., a loop structure, mismatch or hairpin structure).

In certain embodiments, an oligonucleotide provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a COASY nucleic acid, a target region, target segment, or specified portion thereof. Percent complementarity of an oligonucleotide with a target nucleic acid can be determined using routine methods.

For example, an oligonucleotide in which 18 of 20 nucleobases of the oligonucleotide are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, an oligonucleotide which is 18 nucleobases in length having four non-complementary nucleobases which are flanked by two regions of complete complementarity with the target nucleic acid would have 77.8% overall complementarity with the target nucleic acid and would thus fall within the scope of the present invention. Percent complementarity of a oligonucleotide with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, oligonucleotides described herein, or specified portions thereof, are fully complementary (i.e. 100% complementary) to a target nucleic acid, or specified portion thereof. For example, an oligonucleotide may be fully complementary to a COASY nucleic acid, or a target region, or a target segment or target sequence thereof. As used herein, “fully complementary” means each nucleobase of an oligonucleotide is capable of precise base pairing with the corresponding nucleobases of a target nucleic acid. For example, a 20 nucleobase oligonucleotide is fully complementary to a target sequence that is 400 nucleobases long, so long as there is a corresponding 20 nucleobase portion of the target nucleic acid that is fully complementary to the oligonucleotide. Fully complementary can also be used in reference to a specified portion of the first and/or the second nucleic acid. For example, a 20 nucleobase portion of a 30 nucleobase oligonucleotide can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the oligonucleotide. At the same time, the entire 30 nucleobase oligonucleotide may or may not be fully complementary to the target sequence, depending on whether the remaining 10 nucleobases of the oligonucleotide are also complementary to the target sequence.

In certain embodiments, antisense agents described herein comprise one or more mismatched nucleobases relative to the target nucleic acid. In certain such embodiments, antisense activity against the target is reduced by such mismatch, but activity against a non-target is reduced by a greater amount. Thus, in certain such embodiments selectivity of the antisense agent is improved. In certain embodiments, the mismatch is specifically positioned within an oligonucleotide having a gapmer motif. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region. In certain embodiments, the mismatch is specifically positioned within an oligonucleotide not having a gapmer motif. In certain such embodiments, the mismatch is at position 1, 2, 3, 4, 5, 6, 7, 8, 9, 10, 11, or 12 from the 5′-end of the oligonucleotide. In certain such embodiments, the mismatch is at position, 2, 3, 4, 5, 6, 7, 8, 9, 10, 11, or 12 from the 3′-end of the oligonucleotide.

The location of a non-complementary nucleobase may be at the 5′ end or 3′ end of the oligonucleotide. Alternatively, the non-complementary nucleobase or nucleobases may be at an internal position of the oligonucleotide. When two or more non-complementary nucleobases are present, they may be contiguous (i.e. linked) or non-contiguous. In one embodiment, a non-complementary nucleobase is located in the wing segment of a gapmer oligonucleotide.

In certain embodiments, oligonucleotides described herein that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a COASY nucleic acid, or specified portion thereof.

In certain embodiments, oligonucleotides described herein that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a COASY nucleic acid, or specified portion thereof.

In certain embodiments, oligonucleotides described herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an oligonucleotide. In certain embodiments, the oligonucleotides are complementary to at least an 8 nucleobase portion of a target segment. In certain embodiments, the oligonucleotides are complementary to at least a 9 nucleobase portion of a target segment. In certain embodiments, the oligonucleotides are complementary to at least a 10 nucleobase portion of a target segment. In certain embodiments, the oligonucleotides are complementary to at least an 11 nucleobase portion of a target segment. In certain embodiments, the oligonucleotides are complementary to at least a 12 nucleobase portion of a target segment. In certain embodiments, the oligonucleotides are complementary to at least a 13 nucleobase portion of a target segment. In certain embodiments, the oligonucleotides are complementary to at least a 14 nucleobase portion of a target segment. In certain embodiments, the oligonucleotides are complementary to at least a 15 nucleobase portion of a target segment. In certain embodiments, the oligonucleotides are complementary to at least a 16 nucleobase portion of a target segment. Also contemplated are oligonucleotides that are complementary to at least a 9, 10, 11, 12, 13, 14, 15, 16, 17, 18, 19, 20, or more nucleobase portion of a target segment, or a range defined by any two of these values.

### Identity

The oligonucleotides provided herein may also have a defined percent identity to a particular nucleotide sequence, SEQ ID NO, or compound represented by a specific ION number, or portion thereof. An oligonucleotide is identical to a sequence disclosed herein if it has the same nucleobase pairing ability. For example, an RNA which contains uracil in place of thymidine in a disclosed DNA sequence would be considered identical to the DNA sequence since both uracil and thymidine pair with adenine. Shortened and lengthened versions of the oligonucleotides described herein as well as oligonucleotides having non-identical bases relative to the oligonucleotides provided herein also are contemplated. The non-identical bases may be adjacent to each other or dispersed throughout the oligonucleotide. Percent identity of an oligonucleotide is calculated according to the number of bases that have identical base pairing relative to the sequence to which it is being compared.

### Certain Modified Oligonucleotides

In certain embodiments, antisense agents and antisense compounds described herein comprise or consist of oligonucleotides consisting of linked nucleosides. Oligonucleotides may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

### A. Modified Nucleosides

Modified nucleosides comprise a modified sugar moiety or a modified nucleobase or both a modifed sugar moiety and a modified nucleobase.

### 1. ModifiedSugar Moieties

In certain embodiments, sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more acyclic substituent, including but not limited to substituents at the 2′, 4′, and/or 5′ positions. In certain embodiments one or more acyclic substituent of non-bicyclic modified sugar moieties is branched. Examples of 2′-substituent groups suitable for non-bicyclic modified sugar moieties include but are not limited to: F, OCH₃ (“OMe” or “O-methyl”), and O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, —O(CH₂)₂ON(CH₃)₂(“DMAOE”), 2′-O(CH₂)₂O(CH₂)₂N(CH₃)₂(“DMAEOE”), and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for linearlynon-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted nucleoside or 2′—non-bicyclic modified nucleoside comprises a sugar moiety comprising a linear 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′—non-bicyclic modified nucleoside comprises a sugar moiety comprising a linear 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, —O(CH₂)₂ON(CH₃)₂(“DMAOE”), 2′-O(CH₂)₂O(CH₂)₂N(CH₃)₂ (“DMAEOE”), and OCH₂C(═O)—N(H)CH₃ (“NMA”).

In certain embodiments, a 2′-substituted nucleoside or 2′—non-bicyclic modified nucleoside comprises a sugar moiety comprising a linear 2′-substituent group selected from: F, OCH₃, and OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

Nucleosides comprising modified sugar moieties, such as non-bicyclic modified sugar moieties, are referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modifed sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′—CH₂-2′, 4′—(CH₂)₂-2′, 4′—(CH₂)₃-2′, 4′—CH₂—O-2′ (“LNA”), 4′—CH₂—S-2′, 4′—(CH₂)₂—O-2′ (“ENA”), 4′—CH(CH₃)—O-2′ (referred to as “constrained ethyl” or “cEt” when in the S configuration), 4′—CH₂—O—CH₂-2′, 4′—CH₂—N(R)-2′, 4′—CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′—CH₂—N(OCH₃)-2′ and analogs thereof (see, e.g., Prakash et al., U.S. Pat. No. 8,278,425), 4′—CH₂—O—N(CH₃)-2′ (see, e.g., Allerson et al., U.S. Pat. No. 7,696,345 and Allerson et al., U.S. Pat. No. 8,124,745), 4′—CH₂—C(H)(CH₃)-2′ (see, e.g., Zhou, et al., J Org. Chem., 2009, 74, 118-134), 4′—CH₂—C(═CH₂)-2′ and analogs thereof (see e.g., Seth et al., U.S. Pat. No. 8,278,426), 4′—C(RₐR_{b})—N(R)—O-2′, 4′—C(RₐR_{b})—O—N(R)-2′, 4′—CH₂—O—N(R)-2′, and 4′—CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: -[C(Rₐ)(R_{b})ₙ—, -[C(Rₐ)(R_{b})ln-O-, —C(Rₐ)═C(R_{b})—, —C(Rₐ)=N—, —C(═NRₐ)-, —C(═O)—, —C(═S)—, —O—, —Si(Rₐ)₂-, —S(═O)x-, and —N(Rₐ)—;
wherein:
x is 0, 1, or 2;
n is 1,2,3,or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each Ji and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)-H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek etal., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U. S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al.,U.S. Pat. No. 7,053,207, Imanishi et al., U.S. Pat. No. 6,268,490, Imanishi et al. U.S. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 91999/014226; Seth et al.,WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

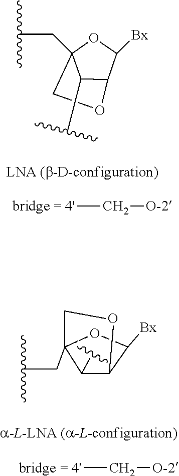

α-L-methyleneoxy (4′—CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see e.g., Leumann, CJ. Bioorg. & Med. Chem. 2002, 10, 841-854), fluoro HNA:

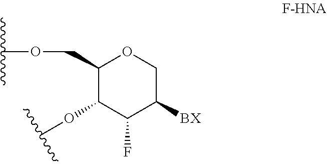

(“F-HNA”, see e.g., Swayze et al., U.S. Pat. No. 8,088,904; Swayze et al., U.S. Pat. No. 8,440,803; Swayze et al., U.S.; and Swayze et al., U.S. Pat. No. 9,005,906, F-HNA can also be referred to as a F-THP or 3′-fluoro tetrahydropyran), and nucleosides comprising additional modified THP compounds having the formula:

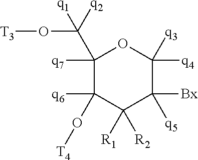

wherein, independently, for each of said modified THP nucleoside: Bx is a nucleobase moiety; T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3-terminal group; q₁, q₂, q₃, q₄, q₅, q₆ and g₇ are each, independently, H, C₁-C₆ alkyl, substituted C₁-C₆ alkyl, C₂- C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is 0, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and g₇ are each H.

In certain embodiments, at least one of q₁, q2, q₃, q₄, q₅, q₆ and q is other than H. In certain embodiments, at least one of q₁, q₂, q₃, q₄, q₅, q₆ and q is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

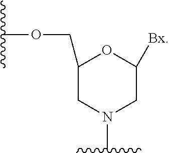

In certain embodiments, morpholinos may be modified, for example by adding or altering various substituent groups from the above morpholino structure. Such sugar surrogates are referred to herein as “modifed morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

### 2. Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to oligonucleotides described herein.

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimi,dines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S.T., Ed., CRC Press, 2008, 163-166 and 442-443.

Publications that teach the preparation of certain of the above noted modified nucleobases as well as other modified nucleobases include without limitation, Manoharan et al., US2003/0158403, Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, modified oligonucleotides comprise one or more modified nucleobases. In certain embodiments, the modified nucleobase is 5-methylcytosine. In certain embodiments, each cytosine is a 5-methylcytosine.

### 3. Modified Internucleoside Linkages

The naturally occurring internucleoside linkage of RNA and DNA is a 3′ to 5′ phosphodiester linkage. In certain embodiments, oligonucleotides described herein having one or more modified, i.e. non-naturally occurring, internucleoside linkages are often selected over oligonucleotides having naturally occurring internucleoside linkages because of desirable properties such as, for example, enhanced cellular uptake, enhanced affinity for target nucleic acids, and increased stability in the presence of nucleases.

In certain embodiments, oligonucleotides comprise one or more modified internucleoside linkages. In certain embodiments, the modified internucleoside linkages are phosphorothioate linkages. In certain embodiments, each internucleoside linkage of the oligonucleotide is a phosphorothioate internucleoside linkage.

In certain embodiments, oligonucleotides having modified internucleoside linkages include internucleoside linkages that retain a phosphorus atom as well as internucleoside linkages that do not have a phosphorus atom.

Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P=O”) (also referred to as unmodified or naturally occurring linkages), phosphotriesters, methylphosphonates, phosphoramidates, and phosphorothioates (“P=S”), and phosphorodithioates (“HS-P=S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH₂—N(CH₃)—O-5′), amide-3 (3′—CH₂—C(═O)—N(H)-5′), amide-4 (3′—CH₂—N(H)—C(═O)-5′), formacetal (3′—O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH2 component parts.

### B. Certain Motifs

In certain embodiments, oligonucleotides can have a motif, e.g. a pattern of unmodified and/or modified sugar moieties, nucleobases, and/or internucleoside linkages. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, antisense agents and antisense compounds described herein comprise oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, the wings of a gapmer comprise 2-5 nucleosides. In certain embodiments, the wings of a gapmer comprise 3-5 nucleosides. In certain embodiments, the nucleosides of a gapmer are all modified nucleosides.

In certain embodiments, the gap of a gapmer comprises 7-12 nucleosides. In certain embodiments, the gap of a gapmer comprises 7-10 nucleosides. In certain embodiments, the gap of a gapmer comprises 8-10 nucleosides. In certain embodiments, the gap of a gapmer comprises 10 nucleosides. In certain embodiment, each nucleoside of the gap of a gapmer is an unmodified 2′-deoxy nucleoside.

In certain embodiments, the gapmer is a deoxy gapmer. In such embodiments, the nucleosides on the gap side of each wing/gap junction are unmodified 2′-deoxy nucleosides and the nucleosides on the wing sides of each wing/gap junction are modified nucleosides. In certain such embodiments, each nucleoside of the gap is an unmodified 2′-deoxy nucleoside. In certain such embodiments, each nucleoside of each wing is a modified nucleoside.

In certain embodiments, a modified oligonucleotide has a fully modified sugar motif wherein each nucleoside of the modified oligonucleotide comprises a modified sugar moiety. In certain embodiments, modified oligonucleotides comprise or consist of a region having a fully modified sugar motif wherein each nucleoside of the region comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

### 2. Certain Nucleobase Motifs

In certain embodiments, antisense agents and antisense compounds described herein comprise oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

### 3. Certain Internucleoside Linkage Motifs

In certain embodiments, antisense agents and antisense compounds described herein comprise oligonucleotides. In certain embodiments, oligonucleotides comprise modified and/or unmodified internucleoside linkages arranged along the oligonucleotide or region thereof in a defined pattern or motif. In certain embodiments, essentially each internucleoside linking group is a phosphodiester internucleoside linkage (P═O). In certain embodiments, each internucleoside linking group of a modified oligonucleotide is a phosphorothioate (P═S) internucleoside linkage. In certain embodiments, each internucleoside linking group of a modified oligonucleotide is independently selected from a phosphorothioate and phosphodiester internucleoside linkage. In certain embodiments, each phosphorothioate internucleoside linkage is independently selected from a stereorandom phosphorothioate, a (Sp) phosphorothioate, and a (Rp) phosphorothioate.

In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified.

### Certain Conjugated Antisense Agents and Antisense Compounds

In certain embodiments, antisense agents and antisense compounds described herein comprise or consist of an oligonucleotide (modified or unmodified) and, optionally, one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides. In certain embodiments, the antisense agent is an RNAi agent comprising a conjugate group. In certain embodiments, the RNAi agent comprises an antisense compound and a sense compound, wherein the sense compound comprises a conjugate group. In certain embodiments, the sense compound comprises a sense oligonucleotide and a conjugate group attached to the sense oligonucleotide. In certain embodiments, the conjugate group is attached to the 3′ end of the sense oligonucleotide.

In certain embodiments, the oligonucleotide is modified. In certain embodiments, the oligonucleotide has a nucleobase sequence that is complementary to a target nucleic acid. In certain embodiments, oligonucleotides are complementary to a messenger RNA (mRNA). In certain embodiments, oligonucleotides are complementary to a pre-mRNA. In certain embodiments, oligonucleotides are complementary to a sense transcript.

Examples of terminal groups include but are not limited to conjugate groups, capping groups, phosphate moieties, protecting groups, modified or unmodified nucleosides, and two or more nucleosides that are independently modified or unmodified.

### Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 1. Conjugate Linkers

Conjugate moieties are attached to oligonucleotides through conjugate linkers. In certain antisense agents and antisense compounds, the conjugate linker is a single chemical bond (i.e., the conjugate moiety is attached directly to an oligonucleotide through a single bond). In certain antisense antisense agents and antisense compounds, a conjugate moiety is attached to an oligonucleotide via a more complex conjugate linker comprising one or more conjugate linker moeities, which are sub-units making up a conjugate linker. In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the compound after it reaches a target tissue. Accordingly, linker-nucleosides are typically linked to one another and to the remainder of the compound through cleavable bonds. In certain embodiments, such cleavable bonds are phosphodiester bonds.

Herein, linker-nucleosides are not considered to be part of the oligonucleotide. Accordingly, in embodiments in which an antisense agent or antisense compound comprises an oligonucleotide consisting of a specified number or range of linked nucleosides and/or a specified percent complementarity to a reference nucleic acid and the antisense agent or antisense compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an antisense agent or antisense compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an antisense agent or antisense compound is more than 30. Alternatively, an antisense agent or antisense compound may comprise a modified oligonucleotide consisting of 8-30 nucleosides and no conjugate group. The total number of contiguous linked nucleosides in such a compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances antisense agents or antisense compounds comprising a particular conjugate moiety are better taken up by a particular cell type, but once the antisense agent or antisense compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the antisense agent or antisense compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### 2. Certain Cell-Targeting Conjugate Moieties

In certain embodiments, a conjugate group comprises a cell-targeting conjugate moiety. In certain embodiments, a conjugate group has the general formula:

$$\underbrace{{[ Ligand - Tether ]}_{n} - {[ Branching group ]}_{m}}_{\begin{matrix}Cell - targeting \\ conjugate moiety\end{matrix}} - \underbrace{{[ \begin{matrix}Conjugate Linker \\ Moiety\end{matrix} ]}_{j} - {[ \begin{matrix}Cleavable Conj . \\ Linker Moiety\end{matrix} ]}_{k}}_{Conjugate Linker} -$$

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate. In certain embodiments, each ligand is, independently selected from galactose, N-acetyl galactoseamine (GalNAc), mannose, glucose, glucoseamine and fucose. In certain embodiments, each ligand is N-acetyl galactoseamine (GalNAc). In certain embodiments, the cell-targeting moiety comprises 3 GalNAc ligands. In certain embodiments, the cell-targeting moiety comprises 2 GalNAc ligands. In certain embodiments, the cell-targeting moiety comprises 1 GalNAc ligand.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29 or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6—O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, conjugate groups comprise a cell-targeting moiety having the formula:

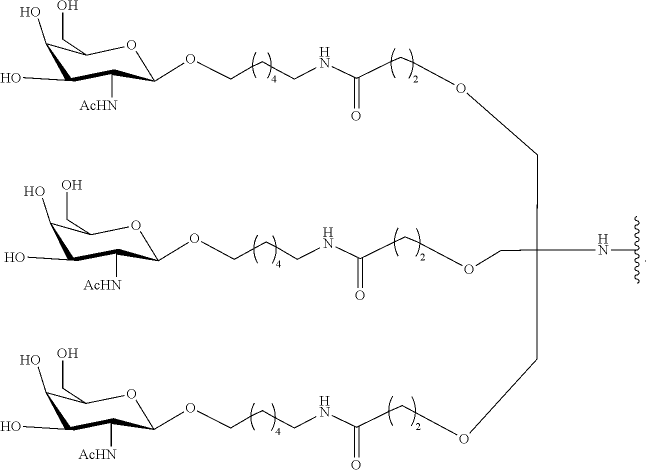

In certain embodiments, conjugate groups comprise a cell-targeting moiety having the formula:

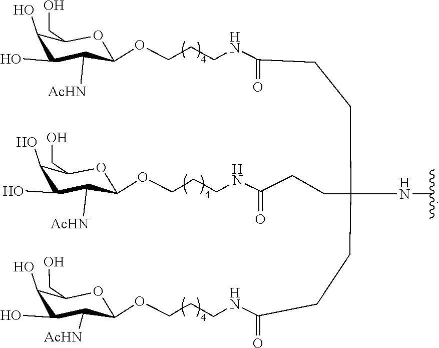

In certain embodiments, conjugate groups comprise a cell-targeting moiety having the formula:

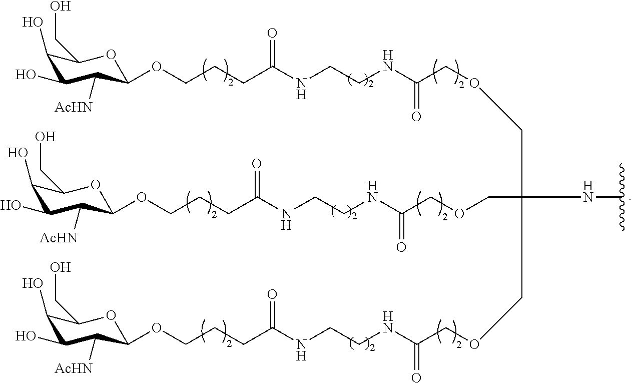

In certain embodiments, conjugate groups comprise a cell-targeting moiety having the formula:

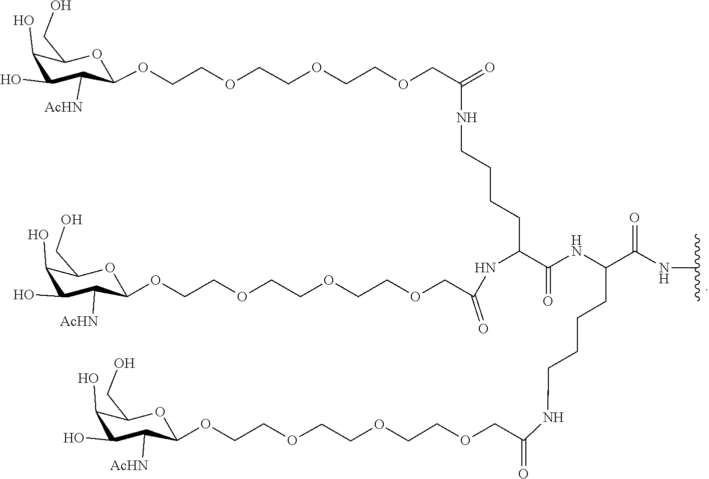

In certain embodiments, conjugate groups comprise a cell-targeting moiety having the formula:

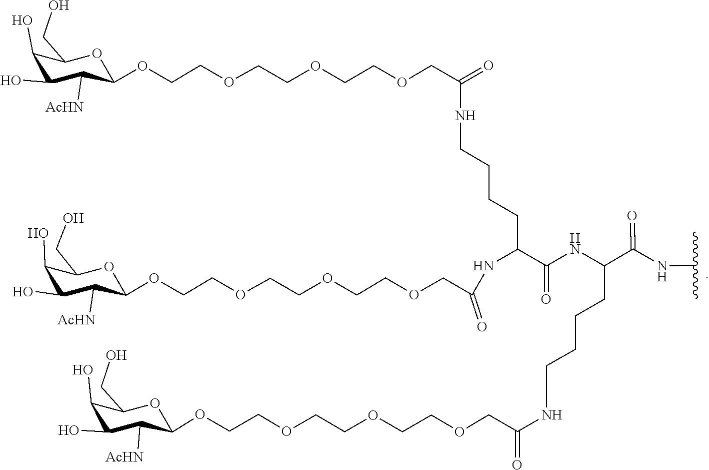

In certain embodiments, antisense agents and antisense compounds comprise a conjugate group described herein as “LICA-1”. LICA-1 has the formula:

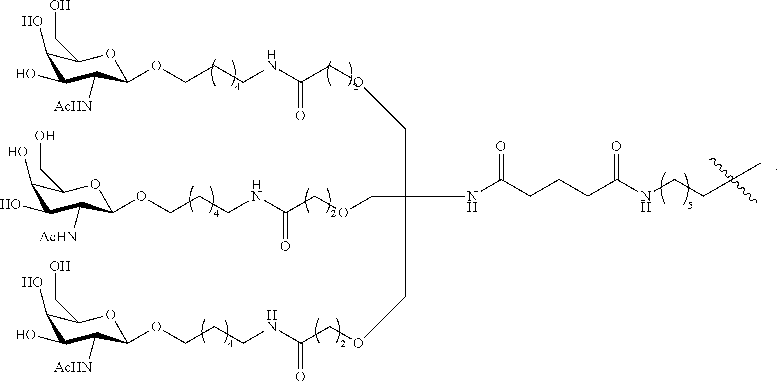

In certain embodiments, antisense agents and antisense compounds described herein comprise LICA-1 and a cleavable moiety within the conjugate linker have the formula:

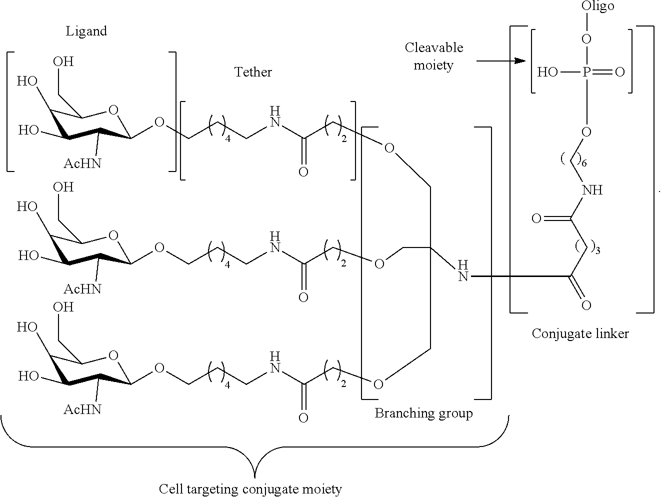

wherein oligo is an oligonucleotide.

Representative United States patents, United States patent application publications, international patent application publications, and other publications that teach the preparation of certain of the above noted conjugate groups, compounds comprising conjugate groups, tethers, conjugate linkers, branching groups, ligands, cleavable moieties as well as other modifications include without limitation, U.S. Pat. Nos. 5,994,517, 6,300,319, 6,660,720, 6,906,182, 7,262,177, 7,491,805, 8,106,022, 7,723,509, US 2006/0148740, US 2011/0123520, WO 2013/033230 and WO 2012/037254, Biessen et al., J. Med. Chem. 1995, 38, 1846-1852, Lee et al., Bioorganic & Medicinal Chemistry 2011,19, 2494-2500, Rensen et al., J Biol. Chem. 2001, 276, 37577-37584, Rensen et al., J. Med. Chem. 2004, 47, 5798-5808, Sliedregt et al., J. Med. Chem. 1999, 42, 609-618, and Valentijn et al., Tetrahedron, 1997, 53, 759-770.

In certain embodiments, modified oligonucleotides comprise a gapmer or fully modified sugar motif and a conjugate group comprising at least one, two, or three GalNAc ligands. In certain embodiments, antisense agents comprise a conjugate group found in any of the following references: Lee, Carbohydr Res, 1978, 67, 509-514; Connolly et al., J Biol Chem, 1982, 257, 939-945; Pavia et al., IntJPep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; 10 Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

In certain embodiments, antisense agents comprising a conjugate group are single-stranded. In certain embodiments, antisense agents comprising a conjugate group are double-stranded.

### Compositions and Methods for Formulating Pharmaceutical Compositions

Antisense agents described herein may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

An antisense agent described herein targeted to a COASY nucleic acid can be utilized in pharmaceutical compositions by combining the antisense agent with a suitable pharmaceutically acceptable diluent or carrier. In certain embodiments, a pharmaceutically acceptable diluent is water, such as sterile water suitable for injection. Accordingly, in one embodiment, employed in the methods described herein is a pharmaceutical composition comprising an antisense agent targeted to a COASY nucleic acid and a pharmaceutically acceptable diluent. In certain embodiments, the pharmaceutically acceptable diluent is water. In certain embodiments, the antisense agent comprises or consists of a modified oligonucleotide provided herein.

Pharmaceutical compositions comprising antisense agents provided herein encompass any pharmaceutically acceptable salts, esters, or salts of such esters, or any other oligonucleotide which, upon administration to a subject, including a human, is capable of providing (directly or indirectly) the biologically active metabolite or residue thereof. Suitable pharmaceutically acceptable salts include, but are not limited to, sodium and potassium salts.

A prodrug can include the incorporation of additional nucleosides at one or both ends of an antinse agent which are cleaved by endogenous nucleases within the body, to form the active compound.

In certain embodiments, the antisense agents or compositions further comprise a pharmaceutically acceptable carrier or diluent.

### Certain Combinations and Combination Therapies

In certain embodiments, an antisense agent described herein is co-administered with one or more secondary agents. In certain embodiments, such second agents are designed to treat the same disease, disorder, or condition as the first agent described herein. In certain embodiments, such second agents are designed to treat a different disease, disorder, or condition as the first agent described herein. In certain embodiments, a first agent is designed to treat an undesired side effect of a second agent. In certain embodiments, second agents are co-administered with the antisense agent to treat an undesired effect of the antisense agent. In certain embodiments, such second agents are designed to treat an undesired side effect of one or more pharmaceutical compositions as described herein. In certain embodiments, second agents are co-administered with the antisense agent to produce a combinational effect. In certain embodiments, second agents are co-administered with the antisense agent to produce a synergistic effect. In certain embodiments, the co-administration of the antisense and second agents permits use of lower dosages than would be required to achieve a therapeutic or prophylactic effect if the agents were administered as independent therapy.

### EXAMPLES

### Non-Limiting Disclosure and Incorporation by Reference

While certain antisense agents, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Predicted Loss of Function Variants in COASY are Associated with Decreased Risk of NAFLD and Decreased Liver Fat Percentage

Variants in COASY were evaluated for associations with non-alcoholic fatty liver disease (NAFLD) and MRI-derived liver fat percentage in approximately 375,000 individuals with genotype data and approximately 45,000 individuals with exome sequencing data from the UK Biobank cohort. Variants evaluated included the rs560987504 COASY frameshift variant as well as a gene burden test which aggregated rs560987504 and several additional rare annotated loss-of-function and predicted damaging missense variants in COASY.

Collectively, predicted loss of function COASY variants were associated with decreased risk of NAFLD in a gene burden test, and the rs560987504 COASY frameshift variant was directionally consistent with decreased risk of NAFLD (Table 1).

TABLE 1
rs560987504and COASY gene burden
association results for NAFLD

| | | | | Non-Alcoholic Fatty | |
|---|---|---|---|---|---|
| | | | | Liver Disease (n = 4129) | |
| rsID | Gene | Function | AAF | P value | OR |
| rs560987504 | COASY | Frameshift | 0.0014 | 0.06 | ↓ 0.495 |
| Burden | COASY | Gene Burden | 0.0029 | 4.88E−03 | ↓ 0.252 |

Additionally, the rs560987504 COASY frameshift variant was associated with decreased MRI-derived liver fat percentage, and collectively this variant and other predicted loss of function COASY variants were associated with decreased MRI-derived liver fat percentage (Table 2). These results indicate that loss-of-function of COASY results in protection from NAFLD and a lower percentage of liver fat.

TABLE 2
rs560987504 and COASY gene burden association
results for liver fat percentage

| | | | | MRI Liver Fat | |
|---|---|---|---|---|---|
| | | | | Percentage (n = 4232) | |
| rsID | Gene | Function | AAF | P value | beta |
| rs560987504 | COASY | Frameshift | 0.0014 | 0.01 | ↓ −0.254 |
| Burden | COASY | Gene Burden | 0.0029 | 3.16E−03 | ↓ −0.172 |

### Example 2: Effect of 3-10-3 cEt Uniform Phosphorothioate Modified Oligonucleotides on Mouse COASY RNA in Vitro, Single Dose

Modified oligonucleotides complementary to mouse COASY nucleic acid were designed and tested for their single dose effects on COASY RNA in vitro. The modified oligonucleotides were tested in a series of experiments that had the same culture conditions.

The modified oligonucleotides in the table below are 3-10-3 cEt modified oligonucleotides with uniform phosphorothioate internucleoside linkages. The modified oligonucleotides are 16 nucleosides in length, wherein the central gap segment consists of ten 2′-β-D-deoxynucleosides, and wherein the 5′ and 3′ wing segments each consist of three cEt nucleosides. The sugar motif for the modified oligonucleotides is (from 5′ to 3′): kkkddddddddddkkk; wherein each “d” represents a 2′-β-D-deoxyribosyl sugar moiety, and each “k” represents a cEt sugar moiety. The internucleoside linkage motif for the modified oligonucleotides is (from 5′ to 3′): sssssssssssssss; wherein each “s” represents a phosphorothioate internucleoside linkage. Each cytosine residue is a 5-methylcytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the table below is 100% complementary to SEQ ID NO: 1 (ENSEMBL Accession No. ENSMUSG00000001755.12 from version 102: November 2020), to SEQ ID NO: 2 (GENBANK Accession No. NM_001305982.1), or to both. “N/A” indicates that the modified oligonucleotide is not 100% complementary to that particular target nucleic acid sequence.

Cultured 4T1 cells were treated with modified oligonucleotide at a concentration of 7000 nM by free uptake at a density of 7,000 cells per well. After a treatment period of approximately 48 hours, total RNA was isolated from the cells and COASY RNA levels were measured by quantitative real-time RTPCR. COASY RNA levels were measured by mouse primer-probe set RTS52828 (forward sequence TGCTTCAGCCTCCAAATGAG, designated herein as SEQ ID NO: 5; reverse sequence TGTATGCTCCCAAGTTCTTCAG, designated herein as SEQ ID NO: 6; probe sequence TCCCGTCAGGTCTCTATGTGCTCG, designated herein as SEQ ID NO: 7). COASY RNA levels were normalized to total RNA content, as measured by RIBOGREEN®. Reduction of COASY RNA is presented in the table below as percent COASY RNA relative to the amount in untreated control cells (% UTC). The values marked with a “T” indicate that the modified oligonucleotide is complementary to the amplicon region of the primer probe set. Additional assays may be used to measure the potency and efficacy of the modified oligonucleotides complementary to the amplicon region.

Each separate experimental analysis described in this example is identified by a letter ID in the table column below labeled “AID” (Analysis ID).

TABLE 3
Reduction of mouse COASY RNA by 3-10-3 cEt modified oligonucleotides with uniform
phosphorothioate internucleoside linkages at a concentration of 7000 nM in 4T1 cells

| | SEQ | SEQ | SEQ | SEQ | | | | |
|---|---|---|---|---|---|---|---|---|
| | ID | ID | ID | ID | | | | |
| | No: 1 | No: 1 | No: 2 | No: 2 | | COASY | | SEQ |
| Compound | Start | Stop | Start | Stop | | (% | | ID |
| Number | Site | Site | Site | Site | Sequence (5' to 3') | UTC) | AID | NO. |
| 1514480 | 1533 | 1548 | N/A | N/A | GACCAATAGGTATCAG | 50 | A | 15 |
| 1514483 | 2140 | 2155 | N/A | N/A | GAGTAAGAGCTAGGAG | 108 | A | 16 |
| 1514485 | 548 | 563 | 240 | 255 | GCACATAGAGGGTGTG | 65 | A | 17 |
| 1514486 | 202 | 217 | N/A | N/A | GCGAAGACTCGGAGGC | 49 | A | 18 |
| 1514487 | 3115 | 3130 | N/A | N/A | GCCAAGGAAGATCCCA | 39 | A | 19 |
| 1514499 | 3956 | 3971 | 2039 | 2054 | AAAAAGCGGACAGAAC | 61 | A | 20 |
| 1514501 | 1241 | 1256 | N/A | N/A | GCATTTTGGAAGGTTG | 9 | A | 21 |
| 1514504 | 2430 | 2445 | N/A | N/A | TCCTAAAGACAGGTAA | 104† | A | 22 |
| 1514505 | 4040 | 4055 | 2123 | 2138 | GCACTTGGAAACATCT | 23 | A | 23 |
| 1514507 | 3245 | 3260 | 1541 | 1556 | CAGCATAGCAGCATCG | 45 | A | 24 |
| 1514510 | 2627 | 2642 | N/A | N/A | GGCTAGTACCTGTTCC | 86 | A | 25 |
| 1514520 | 894 | 909 | 586 | 601 | TAATCCGGATACAGCA | 62 | A | 26 |
| 1514522 | 1385 | 1400 | N/A | N/A | AACCATGAAGTGCAGA | 49 | A | 27 |
| 1514525 | 28 | 43 | 28 | 43 | TCAAACAAAGCATCGC | 60 | A | 28 |
| 1514534 | 50 | 65 | 50 | 65 | GAGCTACGGGAACTCA | 55 | A | 29 |
| 1514536 | 2462 | 2477 | 1200 | 1215 | GCACATAGAGACCTGA | 11† | A | 30 |
| 1514537 | 2709 | 2724 | N/A | N/A | AGAATATCTGGGTGAC | 79 | A | 31 |
| 1514540 | 1499 | 1514 | N/A | N/A | GAACTACTATATTCTT | 36 | A | 32 |
| 1514542 | 1266 | 1281 | N/A | N/A | CAATAACAGAGTGTCA | 19 | A | 33 |
| 1514544 | 1028 | 1043 | 720 | 735 | CAAAAGTGCCACCCAC | 84 | A | 34 |
| 1514549 | 1123 | 1138 | 815 | 830 | CAACAGATCTTTGTCA | 34 | A | 35 |
| 1514553 | 2773 | 2788 | N/A | N/A | GTTTACCTTGTTCCCA | 63 | A | 36 |
| 1514555 | 1527 | 1542 | N/A | N/A | TAGGTATCAGTTGGTG | 8 | A | 37 |
| 1514557 | 3897 | 3912 | 1980 | 1995 | ACAAATTCCAGCATGC | 89 | A | 38 |
| 1514558 | 1335 | 1350 | N/A | N/A | GTTAAAAAGTGACTGA | 22 | A | 39 |
| 1514562 | 1858 | 1873 | 941 | 956 | CCCATAGGGATCCAGC | 123 | A | 40 |
| 1514565 | 3620 | 3635 | N/A | N/A | AACCATTGAACCTTCC | 94 | A | 41 |
| 1514569 | 951 | 966 | 643 | 658 | CTTATAGTAGAGGGTA | 18 | A | 42 |
| 1514572 | 401 | 416 | N/A | N/A | ACGAAGCGAGAATGAC | 83 | A | 43 |
| 1514581 | 2343 | 2358 | N/A | N/A | TCTTACCACGCCCAAC | 62† | A | 44 |
| 1514583 | 3815 | 3830 | 1898 | 1913 | GGGTAAACATCAAGGC | 30 | A | 45 |
| 1514586 | 3201 | 3216 | N/A | N/A | GATACTAGTCACTGCC | 105 | A | 46 |
| 1514587 | 1665 | 1680 | N/A | N/A | CCCAAGCAGTGGCAGC | 43 | A | 47 |
| 1514598 | 3748 | 3763 | 1831 | 1846 | CCATAGAAAATCCACT | 52 | A | 48 |
| 1514600 | 395 | 410 | N/A | N/A | CGAGAATGACTAGGCA | 45 | A | 49 |
| 1514604 | 3960 | 3975 | 2043 | 2058 | CACCAAAAAGCGGACA | 52 | A | 50 |
| 1514611 | 3783 | 3798 | 1866 | 1881 | CATTAGATTTGTTAGT | 58 | A | 51 |
| 1514614 | 3231 | 3246 | 1527 | 1542 | CGATTACACACAGAGT | 16 | A | 52 |
| 1514616 | 2986 | 3001 | 1451 | 1466 | CACAATATCTGTGAGG | 33 | A | 53 |
| 1514629 | 3924 | 3939 | 2007 | 2022 | CCCCATTAGCTGTGTC | 23 | A | 54 |
| 1514636 | 1747 | 1762 | N/A | N/A | GAGCAACTTGCCTGAG | 69 | A | 55 |
| 1514644 | 3715 | 3730 | 1798 | 1813 | TAAGCCTTAGGGAGGC | 69 | A | 56 |
| 1514645 | 3367 | 3382 | N/A | N/A | GATCAGAGCAGGAGAC | 114 | A | 57 |
| 1514651 | 2060 | 2075 | N/A | N/A | ACAAGACAAGTTCTTC | 64 | A | 58 |
| 1514653 | 3651 | 3666 | N/A | N/A | TGATACAAGAAAAGGC | 92 | A | 59 |
| 1514656 | 2128 | 2143 | N/A | N/A | GGAGAAGGCATCTGGA | 109 | A | 60 |
| 1514661 | 2272 | 2287 | 1131 | 1146 | GGCGAAAGCTGGAGGA | 76 | A | 61 |
| 1514664 | 17 | 32 | 17 | 32 | ATCGCCGAGAGACTCC | 114 | A | 62 |
| 1514669 | 2834 | 2849 | N/A | N/A | CCAGACTAGGGAGTGC | 67 | A | 63 |
| 1514682 | 3625 | 3640 | N/A | N/A | AAGCTAACCATTGAAC | 67 | A | 64 |
| 1514684 | 1308 | 1323 | N/A | N/A | AAGATACCACATGTAT | 26 | A | 65 |
| 1514688 | 312 | 327 | N/A | N/A | CGCTGATAGAAACCGG | 67 | A | 66 |
| 1514698 | 446 | 461 | 138 | 153 | AGCGGAACACGGCCAT | 78 | A | 67 |
| 1514704 | 1272 | 1287 | N/A | N/A | CCTTAGCAATAACAGA | 31 | A | 68 |
| 1514705 | 817 | 832 | 509 | 524 | TTTAACAGGGTTGTAC | 58 | A | 69 |
| 1514711 | 1314 | 1329 | N/A | N/A | AGACTAAAGATACCAC | 30 | A | 70 |
| 1514719 | 3693 | 3708 | 1776 | 1791 | GTAGAAGATTCCACGC | 85 | A | 71 |
| 1514720 | 1592 | 1607 | N/A | N/A | CACTTTGAGAGCTGAC | 24 | A | 72 |
| 1514725 | 849 | 864 | 541 | 556 | TAACAACTGGTGGCAT | 43 | A | 73 |
| 1514736 | 3983 | 3998 | 2066 | 2081 | CACTAGGTAGACATCA | 43 | A | 74 |
| 1514737 | 4023 | 4038 | 2106 | 2121 | TAATTCAGTTACTGGG | 13 | A | 75 |
| 1514740 | 1410 | 1425 | N/A | N/A | AAACCTCCGAGCCGAT | 50 | A | 76 |
| 1514747 | 3842 | 3857 | 1925 | 1940 | AGCTTAAAGCCCCTGA | 45 | A | 77 |
| 1514748 | 3811 | 3826 | 1894 | 1909 | AAACATCAAGGCCCCC | 65 | A | 78 |
| 1514752 | 1562 | 1577 | N/A | N/A | CAAGTTAAGAAATGCC | 59 | A | 79 |
| 1514762 | 3993 | 4008 | 2076 | 2091 | AACCCATCAACACTAG | 88 | A | 80 |
| 1514766 | 624 | 639 | 316 | 331 | AGAACCTCAAATGTGG | 51 | A | 81 |
| 1514767 | 3593 | 3608 | N/A | N/A | CTAACCTAGTCTTTCC | 88 | A | 82 |
| 1514772 | 1489 | 1504 | N/A | N/A | ATTCTTAGAGTCCCCT | 21 | A | 83 |
| 1514777 | 2506 | 2521 | 1244 | 1259 | AGCTACCGAGCTTTTC | 30† | A | 84 |
| 1514778 | 3822 | 3837 | 1905 | 1920 | AGATAGAGGGTAAACA | 76 | A | 85 |
| 1514780 | 2924 | 2939 | N/A | N/A | TTAACTTTCCTGAGAA | 109 | A | 86 |
| 1514781 | 3838 | 3853 | 1921 | 1936 | TAAAGCCCCTGAGGCC | 109 | A | 87 |
| 1514782 | 2680 | 2695 | N/A | N/A | TAAGCAAAGAGGTCAC | 109 | A | 88 |
| 1514785 | 899 | 914 | 591 | 606 | TTCCATAATCCGGATA | 60 | A | 89 |
| 1514787 | 691 | 706 | 383 | 398 | CAGTAGAATTCTGACG | 56 | A | 90 |
| 1514478 | 399 | 414 | N/A | N/A | GAAGCGAGAATGACTA | 47 | B | 91 |
| 1514479 | 3841 | 3856 | 1924 | 1939 | GCTTAAAGCCCCTGAG | 101 | B | 92 |
| 1514497 | 848 | 863 | 540 | 555 | AACAACTGGTGGCATA | 42 | B | 93 |
| 1514498 | 3825 | 3840 | 1908 | 1923 | GCCAGATAGAGGGTAA | 51 | B | 94 |
| 1514500 | 3600 | 3615 | N/A | N/A | CCACAACCTAACCTAG | 113 | B | 95 |
| 1514509 | 3692 | 3707 | 1775 | 1790 | TAGAAGATTCCACGCC | 71 | B | 96 |
| 1514517 | 1509 | 1524 | N/A | N/A | GAACCCCCAAGAACTA | 39 | B | 97 |
| 1514527 | 3786 | 3801 | 1869 | 1884 | CACCATTAGATTTGTT | 28 | B | 98 |
| 1514528 | 2735 | 2750 | 1389 | 1404 | TCCTATTGATGGTGCC | 54 | B | 99 |
| 1514530 | 1027 | 1042 | 719 | 734 | AAAAGTGCCACCCACG | 89 | B | 100 |
| 1514535 | 3821 | 3836 | 1904 | 1919 | GATAGAGGGTAAACAT | 31 | B | 101 |
| 1514539 | 3314 | 3329 | N/A | N/A | AGATACCTCAGTCTCA | 95 | B | 102 |
| 1514541 | 3751 | 3766 | 1834 | 1849 | GCGCCATAGAAAATCC | 54 | B | 103 |
| 1514551 | 1307 | 1322 | N/A | N/A | AGATACCACATGTATT | 28 | B | 104 |
| 1514559 | 2421 | 2436 | N/A | N/A | CAGGTAAGAGGGAACC | 120† | B | 105 |
| 1514561 | 3921 | 3936 | 2004 | 2019 | CATTAGCTGTGTCCTC | 12 | B | 106 |
| 1514563 | 1240 | 1255 | N/A | N/A | CATTTTGGAAGGTTGA | 17 | B | 107 |
| 1514566 | 197 | 212 | N/A | N/A | GACTCGGAGGCGAGGG | 106 | B | 108 |
| 1514570 | 1494 | 1509 | N/A | N/A | ACTATATTCTTAGAGT | 97 | B | 109 |
| 1514571 | 311 | 326 | N/A | N/A | GCTGATAGAAACCGGA | 55 | B | 110 |
| 1514575 | 3230 | 3245 | 1526 | 1541 | GATTACACACAGAGTT | 41 | B | 111 |
| 1514576 | 3243 | 3258 | 1539 | 1554 | GCATAGCAGCATCGAT | 87 | B | 112 |
| 1514585 | 2067 | 2082 | N/A | N/A | GAACTTGACAAGACAA | 93 | B | 113 |
| 1514589 | 1332 | 1347 | N/A | N/A | AAAAAGTGACTGAACC | 57 | B | 114 |
| 1514591 | 391 | 406 | N/A | N/A | AATGACTAGGCAGGCT | 66 | B | 115 |
| 1514595 | 3714 | 3729 | 1797 | 1812 | AAGCCTTAGGGAGGCG | 77 | B | 116 |
| 1514599 | 49 | 64 | 49 | 64 | AGCTACGGGAACTCAG | 58 | B | 117 |
| 1514601 | 3624 | 3639 | N/A | N/A | AGCTAACCATTGAACC | 117 | B | 118 |
| 1514602 | 2985 | 3000 | 1450 | 1465 | ACAATATCTGTGAGGA | 13 | B | 119 |
| 1514609 | 634 | 649 | 326 | 341 | GATGAAATCCAGAACC | 70 | B | 120 |
| 1514610 | 2678 | 2693 | N/A | N/A | AGCAAAGAGGTCACAC | 122 | B | 121 |
| 1514613 | 1561 | 1576 | N/A | N/A | AAGTTAAGAAATGCCT | 51 | B | 122 |
| 1514618 | 890 | 905 | 582 | 597 | CCGGATACAGCAACAC | 81 | B | 123 |
| 1514619 | 3986 | 4001 | 2069 | 2084 | CAACACTAGGTAGACA | 30 | B | 124 |
| 1514621 | 1269 | 1284 | N/A | N/A | TAGCAATAACAGAGTG | 28 | B | 125 |
| 1514627 | 3592 | 3607 | N/A | N/A | TAACCTAGTCTTTCCC | 103 | B | 126 |
| 1514632 | 2059 | 2074 | N/A | N/A | CAAGACAAGTTCTTCC | 99 | B | 127 |
| 1514633 | 2333 | 2348 | N/A | N/A | CCCAACCAGCGAGGAG | 62† | B | 128 |
| 1514637 | 3884 | 3899 | 1967 | 1982 | TGCTAGGCGGACTTCC | 23 | B | 129 |
| 1514640 | 3996 | 4011 | 2079 | 2094 | CCAAACCCATCAACAC | 71 | B | 130 |
| 1514641 | 3955 | 3970 | 2038 | 2053 | AAAAGCGGACAGAACC | 100 | B | 131 |
| 1514642 | 1487 | 1502 | N/A | N/A | TCTTAGAGTCCCCTCA | 28 | B | 132 |
| 1514643 | 620 | 635 | 312 | 327 | CCTCAAATGTGGCCTG | 86 | B | 133 |
| 1514650 | 2708 | 2723 | N/A | N/A | GAATATCTGGGTGACA | 62 | B | 134 |
| 1514654 | 2832 | 2847 | N/A | N/A | AGACTAGGGAGTGCAC | 89 | B | 135 |
| 1514658 | 1265 | 1280 | N/A | N/A | AATAACAGAGTGTCAG | 16 | B | 136 |
| 1514659 | 2625 | 2640 | N/A | N/A | CTAGTACCTGTTCCAA | 98 | B | 137 |
| 1514673 | 4028 | 4043 | 2111 | 2126 | ATCTTTAATTCAGTTA | 9 | B | 138 |
| 1514674 | 2461 | 2476 | 1199 | 1214 | CACATAGAGACCTGAC | 11† | B | 139 |
| 1514676 | 1031 | 1046 | 723 | 738 | GGTCAAAAGTGCCACC | 83 | B | 140 |
| 1514679 | 3959 | 3974 | 2042 | 2057 | ACCAAAAAGCGGACAG | 35 | B | 141 |
| 1514689 | 2504 | 2519 | 1242 | 1257 | CTACCGAGCTTTTCCC | 7† | B | 142 |
| 1514692 | 3632 | 3647 | N/A | N/A | GTCAGAAAAGCTAACC | 98 | B | 143 |
| 1514699 | 442 | 457 | 134 | 149 | GAACACGGCCATGCTC | 86 | B | 144 |
| 1514709 | 3200 | 3215 | N/A | N/A | ATACTAGTCACTGCCA | 88 | B | 145 |
| 1514718 | 3979 | 3994 | 2062 | 2077 | AGGTAGACATCACATC | 25 | B | 146 |
| 1514723 | 1313 | 1328 | N/A | N/A | GACTAAAGATACCACA | 54 | B | 147 |
| 1514724 | 27 | 42 | 27 | 42 | CAAACAAAGCATCGCC | 90 | B | 148 |
| 1514728 | 3747 | 3762 | 1830 | 1845 | CATAGAAAATCCACTG | 30 | B | 149 |
| 1514732 | 2271 | 2286 | 1130 | 1145 | GCGAAAGCTGGAGGAG | 70 | B | 150 |
| 1514734 | 1726 | 1741 | N/A | N/A | AGCGAGGAACGGTGAG | 42 | B | 151 |
| 1514735 | 816 | 831 | 508 | 523 | TTAACAGGGTTGTACT | 66 | B | 152 |
| 1514742 | 1532 | 1547 | N/A | N/A | ACCAATAGGTATCAGT | 25 | B | 153 |
| 1514744 | 1358 | 1373 | N/A | N/A | CCATTTGGGAAGTTCT | 68 | B | 154 |
| 1514746 | 1388 | 1403 | N/A | N/A | GACAACCATGAAGTGC | 21 | B | 155 |
| 1514755 | 1591 | 1606 | N/A | N/A | ACTTTGAGAGCTGACG | 34 | B | 156 |
| 1514756 | 950 | 965 | 642 | 657 | TTATAGTAGAGGGTAA | 37 | B | 157 |
| 1514758 | 1608 | 1623 | N/A | N/A | GAAAACAAGCATCAGT | 83 | B | 158 |
| 1514760 | 897 | 912 | 589 | 604 | CCATAATCCGGATACA | 47 | B | 159 |
| 1514761 | 547 | 562 | 239 | 254 | CACATAGAGGGTGTGA | 87 | B | 160 |
| 1514763 | 2139 | 2154 | N/A | N/A | AGTAAGAGCTAGGAGA | 73 | B | 161 |
| 1514770 | 2914 | 2929 | N/A | N/A | TGAGAAATACAGCTGT | 82 | B | 162 |
| 1514771 | 3814 | 3829 | 1897 | 1912 | GGTAAACATCAAGGCC | 92 | B | 163 |
| 1514773 | 1857 | 1872 | 940 | 955 | CCATAGGGATCCAGCA | 111 | B | 164 |
| 1514783 | 3109 | 3124 | N/A | N/A | GAAGATCCCAGAGGGT | 84 | B | 165 |
| 1514481 | 1387 | 1402 | N/A | N/A | ACAACCATGAAGTGCA | 24 | C | 166 |
| 1514482 | 1232 | 1247 | N/A | N/A | AAGGTTGAGAAGTGGC | 9 | C | 167 |
| 1514484 | 3541 | 3556 | 1727 | 1742 | CCACAAGGTGCTCAGA | 24 | C | 168 |
| 1514491 | 2458 | 2473 | 1196 | 1211 | ATAGAGACCTGACGGG | 12† | C | 169 |
| 1514496 | 3963 | 3978 | 2046 | 2061 | CACCACCAAAAAGCGG | 56 | C | 170 |
| 1514511 | 1310 | 1325 | N/A | N/A | TAAAGATACCACATGT | 29 | C | 171 |
| 1514516 | 948 | 963 | 640 | 655 | ATAGTAGAGGGTAAGA | 21 | C | 172 |
| 1514519 | 1030 | 1045 | 722 | 737 | GTCAAAAGTGCCACCC | 37 | C | 173 |
| 1514521 | 310 | 325 | N/A | N/A | CTGATAGAAACCGGAA | 32 | C | 174 |
| 1514523 | 3813 | 3828 | 1896 | 1911 | GTAAACATCAAGGCCC | 53 | C | 175 |
| 1514526 | 1264 | 1279 | N/A | N/A | ATAACAGAGTGTCAGA | 17 | C | 176 |
| 1514538 | 3820 | 3835 | 1903 | 1918 | ATAGAGGGTAAACATC | 81 | C | 177 |
| 1514546 | 3947 | 3962 | 2030 | 2045 | ACAGAACCTCAGAGCG | 28 | C | 178 |
| 1514547 | 819 | 834 | 511 | 526 | TGTTTAACAGGGTTGT | 26 | C | 179 |
| 1514556 | 2682 | 2697 | N/A | N/A | GCTAAGCAAAGAGGTC | 102 | C | 180 |
| 1514564 | 3237 | 3252 | 1533 | 1548 | CAGCATCGATTACACA | 26 | C | 181 |
| 1514577 | 700 | 715 | 392 | 407 | GATATTGGTCAGTAGA | 17 | C | 182 |
| 1514580 | 3313 | 3328 | N/A | N/A | GATACCTCAGTCTCAG | 91 | C | 183 |
| 1514590 | 1306 | 1321 | N/A | N/A | GATACCACATGTATTC | 47 | C | 184 |
| 1514594 | 3206 | 3221 | N/A | N/A | GAGGAGATACTAGTCA | 46 | C | 185 |
| 1514596 | 1268 | 1283 | N/A | N/A | AGCAATAACAGAGTGT | 16 | C | 186 |
| 1514603 | 3596 | 3611 | N/A | N/A | AACCTAACCTAGTCTT | 53 | C | 187 |
| 1514607 | 1573 | 1588 | N/A | N/A | TACTCCAAGAACAAGT | 53 | C | 188 |
| 1514608 | 1331 | 1346 | N/A | N/A | AAAAGTGACTGAACCC | 28 | C | 189 |
| 1514612 | 1337 | 1352 | N/A | N/A | CAGTTAAAAAGTGACT | 79 | C | 190 |
| 1514615 | 3899 | 3914 | 1982 | 1997 | GGACAAATTCCAGCAT | 78 | C | 191 |
| 1514617 | 3745 | 3760 | 1828 | 1843 | TAGAAAATCCACTGTC | 26 | C | 192 |
| 1514620 | 632 | 647 | 324 | 339 | TGAAATCCAGAACCTC | 73 | C | 193 |
| 1514624 | 896 | 911 | 588 | 603 | CATAATCCGGATACAG | 36 | C | 194 |
| 1514628 | 3627 | 3642 | N/A | N/A | AAAAGCTAACCATTGA | 79 | C | 195 |
| 1514630 | 398 | 413 | N/A | N/A | AAGCGAGAATGACTAG | 39 | C | 196 |
| 1514634 | 2062 | 2077 | N/A | N/A | TGACAAGACAAGTTCT | 92 | C | 197 |
| 1514639 | 1607 | 1622 | N/A | N/A | AAAACAAGCATCAGTC | 74 | C | 198 |
| 1514647 | 3713 | 3728 | 1796 | 1811 | AGCCTTAGGGAGGCGC | 117 | C | 199 |
| 1514648 | 2134 | 2149 | N/A | N/A | GAGCTAGGAGAAGGCA | 56 | C | 200 |
| 1514655 | 3658 | 3673 | N/A | N/A | GCCAGAATGATACAAG | 49 | C | 201 |
| 1514657 | 2270 | 2285 | 1129 | 1144 | CGAAAGCTGGAGGAGC | 57 | C | 202 |
| 1514660 | 2332 | 2347 | N/A | N/A | CCAACCAGCGAGGAGG | 55† | C | 203 |
| 1514662 | 2389 | 2404 | N/A | N/A | TCAAAAGCCCAGCTTC | 121† | C | 204 |
| 1514663 | 3824 | 3839 | 1907 | 1922 | CCAGATAGAGGGTAAA | 47 | C | 205 |
| 1514665 | 3623 | 3638 | N/A | N/A | GCTAACCATTGAACCT | 67 | C | 206 |
| 1514667 | 1531 | 1546 | N/A | N/A | CCAATAGGTATCAGTT | 11 | C | 207 |
| 1514671 | 2469 | 2484 | 1207 | 1222 | AGCCCGAGCACATAGA | 9† | C | 208 |
| 1514677 | 1501 | 1516 | N/A | N/A | AAGAACTACTATATTC | 43 | C | 209 |
| 1514680 | 3958 | 3973 | 2041 | 2056 | CCAAAAAGCGGACAGA | 64 | C | 210 |
| 1514683 | 3840 | 3855 | 1923 | 1938 | CTTAAAGCCCCTGAGG | 56 | C | 211 |
| 1514687 | 3846 | 3861 | 1929 | 1944 | GCTTAGCTTAAAGCCC | 44 | C | 212 |
| 1514690 | 404 | 419 | N/A | N/A | GAGACGAAGCGAGAAT | 55 | C | 213 |
| 1514691 | 953 | 968 | 645 | 660 | GCCTTATAGTAGAGGG | 44 | C | 214 |
| 1514696 | 853 | 868 | 545 | 560 | GCTGTAACAACTGGTG | 33 | C | 215 |
| 1514697 | 2733 | 2748 | 1387 | 1402 | CTATTGATGGTGCCAT | 45 | C | 216 |
| 1514700 | 2713 | 2728 | N/A | N/A | ATGGAGAATATCTGGG | 49 | C | 217 |
| 1514701 | 3995 | 4010 | 2078 | 2093 | CAAACCCATCAACACT | 104 | C | 218 |
| 1514706 | 2984 | 2999 | 1449 | 1464 | CAATATCTGTGAGGAT | 37 | C | 219 |
| 1514707 | 3153 | 3168 | N/A | N/A | ACCAAATGCTGTCCTA | 30 | C | 220 |
| 1514708 | 119 | 134 | N/A | N/A | CTACTTACACCGCCGT | 35 | C | 221 |
| 1514712 | 4027 | 4042 | 2110 | 2125 | TCTTTAATTCAGTTAC | 14 | C | 222 |
| 1514713 | 1447 | 1462 | N/A | N/A | CCCCAATCATCTCCTA | 27 | C | 223 |
| 1514714 | 1493 | 1508 | N/A | N/A | CTATATTCTTAGAGTC | 34 | C | 224 |
| 1514721 | 2573 | 2588 | 1311 | 1326 | GAGCATAGGCCCGATG | 104 | C | 225 |
| 1514722 | 388 | 403 | N/A | N/A | GACTAGGCAGGCTGAG | 60 | C | 226 |
| 1514726 | 3785 | 3800 | 1868 | 1883 | ACCATTAGATTTGTTA | 18 | C | 227 |
| 1514730 | 2677 | 2692 | N/A | N/A | GCAAAGAGGTCACACC | 56 | C | 228 |
| 1514731 | 815 | 830 | 507 | 522 | TAACAGGGTTGTACTG | 41 | C | 229 |
| 1514733 | 3001 | 3016 | 1466 | 1481 | TGCGATAACTGGCCAC | 56 | C | 230 |
| 1514750 | 25 | 40 | 25 | 40 | AACAAAGCATCGCCGA | 79 | C | 231 |
| 1514753 | 2800 | 2815 | N/A | N/A | ATCTCAAGAACTCTTG | 63 | C | 232 |
| 1514757 | 48 | 63 | 48 | 63 | GCTACGGGAACTCAGA | 43 | C | 233 |
| 1514759 | 1723 | 1738 | N/A | N/A | GAGGAACGGTGAGATC | 84 | C | 234 |
| 1514765 | 3985 | 4000 | 2068 | 2083 | AACACTAGGTAGACAT | 34 | C | 235 |
| 1514775 | 1560 | 1575 | N/A | N/A | AGTTAAGAAATGCCTA | 103 | C | 236 |
| 1514776 | 3750 | 3765 | 1833 | 1848 | CGCCATAGAAAATCCA | 52 | C | 237 |
| 1514784 | 2857 | 2872 | N/A | N/A | TAAGCAGAGACTGACT | 43 | C | 238 |
| 1514786 | 619 | 634 | 311 | 326 | CTCAAATGTGGCCTGC | 100 | C | 239 |
| 1514789 | 1837 | 1852 | 920 | 935 | GACGAGTTCAAAGGTC | 121 | C | 240 |
| 581642 | 1336 | 1351 | N/A | N/A | AGTTAAAAAGTGACTG | 44 | D | 241 |
| 1514488 | 1316 | 1331 | N/A | N/A | CCAGACTAAAGATACC | 21 | D | 242 |
| 1514489 | 3202 | 3217 | N/A | N/A | AGATACTAGTCACTGC | 75 | D | 243 |
| 1514492 | 3823 | 3838 | 1906 | 1921 | CAGATAGAGGGTAAAC | 37 | D | 244 |
| 1514493 | 2344 | 2359 | N/A | N/A | ATCTTACCACGCCCAA | 58† | D | 245 |
| 1514494 | 3941 | 3956 | 2024 | 2039 | CCTCAGAGCGGATCGC | 34 | D | 246 |
| 1514495 | 2791 | 2806 | N/A | N/A | ACTCTTGAAGATGTGT | 61 | D | 247 |
| 1514502 | 3957 | 3972 | 2040 | 2055 | CAAAAAGCGGACAGAA | 37 | D | 248 |
| 1514503 | 939 | 954 | 631 | 646 | GGTAAGAGGGCATTCG | 2 | D | 249 |
| 1514508 | 29 | 44 | 29 | 44 | ATCAAACAAAGCATCG | 56 | D | 250 |
| 1514512 | 1309 | 1324 | N/A | N/A | AAAGATACCACATGTA | 21 | D | 251 |
| 1514513 | 3812 | 3827 | 1895 | 1910 | TAAACATCAAGGCCCC | 65 | D | 252 |
| 1514514 | 3818 | 3833 | 1901 | 1916 | AGAGGGTAAACATCAA | 45 | D | 253 |
| 1514515 | 2463 | 2478 | 1201 | 1216 | AGCACATAGAGACCTG | 23† | D | 254 |
| 1514518 | 2931 | 2946 | N/A | N/A | GCAGAGGTTAACTTTC | 41 | D | 255 |
| 1514524 | 4024 | 4039 | 2107 | 2122 | TTAATTCAGTTACTGG | 19 | D | 256 |
| 1514529 | 1490 | 1505 | N/A | N/A | TATTCTTAGAGTCCCC | 15 | D | 257 |
| 1514531 | 3843 | 3858 | 1926 | 1941 | TAGCTTAAAGCCCCTG | 21 | D | 258 |
| 1514532 | 1605 | 1620 | N/A | N/A | AACAAGCATCAGTCAC | 52 | D | 259 |
| 1514533 | 3626 | 3641 | N/A | N/A | AAAGCTAACCATTGAA | 77 | D | 260 |
| 1514543 | 2061 | 2076 | N/A | N/A | GACAAGACAAGTTCTT | 53 | D | 261 |
| 1514545 | 397 | 412 | N/A | N/A | AGCGAGAATGACTAGG | 29 | D | 262 |
| 1514548 | 1942 | 1957 | 1025 | 1040 | GCGGAAGCGGTTGACA | 48 | D | 263 |
| 1514550 | 2681 | 2696 | N/A | N/A | CTAAGCAAAGAGGTCA | 97 | D | 264 |
| 1514552 | 117 | 132 | N/A | N/A | ACTTACACCGCCGTGG | 92 | D | 265 |
| 1514554 | 2320 | 2335 | N/A | N/A | GAGGAAGGCTTACATT | 79† | D | 266 |
| 1514560 | 1500 | 1515 | N/A | N/A | AGAACTACTATATTCT | 47 | D | 267 |
| 1514567 | 699 | 714 | 391 | 406 | ATATTGGTCAGTAGAA | 45 | D | 268 |
| 1514568 | 2854 | 2869 | N/A | N/A | GCAGAGACTGACTTCC | 26 | D | 269 |
| 1514573 | 3743 | 3758 | 1826 | 1841 | GAAAATCCACTGTCAG | 11 | D | 270 |
| 1514574 | 3994 | 4009 | 2077 | 2092 | AAACCCATCAACACTA | 82 | D | 271 |
| 1514578 | 450 | 465 | 142 | 157 | CCTGAGCGGAACACGG | 54 | D | 272 |
| 1514579 | 3413 | 3428 | N/A | N/A | CAATGGATGACACACG | 105 | D | 273 |
| 1514584 | 3784 | 3799 | 1867 | 1882 | CCATTAGATTTGTTAG | 6 | D | 274 |
| 1514592 | 1267 | 1282 | N/A | N/A | GCAATAACAGAGTGTC | 8 | D | 275 |
| 1514593 | 1029 | 1044 | 721 | 736 | TCAAAAGTGCCACCCA | 50 | D | 276 |
| 1514605 | 626 | 641 | 318 | 333 | CCAGAACCTCAAATGT | 27 | D | 277 |
| 1514606 | 1571 | 1586 | N/A | N/A | CTCCAAGAACAAGTTA | 30 | D | 278 |
| 1514622 | 3898 | 3913 | 1981 | 1996 | GACAAATTCCAGCATG | 38 | D | 279 |
| 1514623 | 402 | 417 | N/A | N/A | GACGAAGCGAGAATGA | 49 | D | 280 |
| 1514625 | 818 | 833 | 510 | 525 | GTTTAACAGGGTTGTA | 12 | D | 281 |
| 1514626 | 3961 | 3976 | 2044 | 2059 | CCACCAAAAAGCGGAC | 39 | D | 282 |
| 1514635 | 2431 | 2446 | N/A | N/A | CTCCTAAAGACAGGTA | 57† | D | 283 |
| 1514646 | 3622 | 3637 | N/A | N/A | CTAACCATTGAACCTT | 55 | D | 284 |
| 1514649 | 3695 | 3710 | 1778 | 1793 | CTGTAGAAGATTCCAC | 33 | D | 285 |
| 1514652 | 2571 | 2586 | 1309 | 1324 | GCATAGGCCCGATGGC | 109 | D | 286 |
| 1514668 | 23 | 38 | 23 | 38 | CAAAGCATCGCCGAGA | 49 | D | 287 |
| 1514672 | 852 | 867 | 544 | 559 | CTGTAACAACTGGTGG | 24 | D | 288 |
| 1514675 | 952 | 967 | 644 | 659 | CCTTATAGTAGAGGGT | 50 | D | 289 |
| 1514678 | 1529 | 1544 | N/A | N/A | AATAGGTATCAGTTGG | 6 | D | 290 |
| 1514681 | 2141 | 2156 | N/A | N/A | GGAGTAAGAGCTAGGA | 45 | D | 291 |
| 1514685 | 2675 | 2690 | N/A | N/A | AAAGAGGTCACACCAT | 49 | D | 292 |
| 1514686 | 3657 | 3672 | N/A | N/A | CCAGAATGATACAAGA | 81 | D | 293 |
| 1514693 | 2133 | 2148 | N/A | N/A | AGCTAGGAGAAGGCAT | 100 | D | 294 |
| 1514695 | 214 | 229 | N/A | N/A | AACCGGCCATCTGCGA | 34 | D | 295 |
| 1514702 | 1273 | 1288 | N/A | N/A | CCCTTAGCAATAACAG | 15 | D | 296 |
| 1514703 | 349 | 364 | N/A | N/A | GACTCAAGAGGGACCT | 90 | D | 297 |
| 1514710 | 3749 | 3764 | 1832 | 1847 | GCCATAGAAAATCCAC | 20 | D | 298 |
| 1514715 | 1386 | 1401 | N/A | N/A | CAACCATGAAGTGCAG | 39 | D | 299 |
| 1514716 | 3233 | 3248 | 1529 | 1544 | ATCGATTACACACAGA | 55 | D | 300 |
| 1514717 | 3594 | 3609 | N/A | N/A | CCTAACCTAGTCTTTC | 48 | D | 301 |
| 1514727 | 2710 | 2725 | N/A | N/A | GAGAATATCTGGGTGA | 82 | D | 302 |
| 1514729 | 549 | 564 | 241 | 256 | TGCACATAGAGGGTGT | 61 | D | 303 |
| 1514738 | 2999 | 3014 | 1464 | 1479 | CGATAACTGGCCACAC | 73 | D | 304 |
| 1514741 | 895 | 910 | 587 | 602 | ATAATCCGGATACAGC | 53 | D | 305 |
| 1514743 | 1772 | 1787 | 855 | 870 | CTGCATACGGCTGGAG | 57 | D | 306 |
| 1514745 | 3839 | 3854 | 1922 | 1937 | TTAAAGCCCCTGAGGC | 66 | D | 307 |
| 1514749 | 1559 | 1574 | N/A | N/A | GTTAAGAAATGCCTAC | 92 | D | 308 |
| 1514751 | 1263 | 1278 | N/A | N/A | TAACAGAGTGTCAGAC | 26 | D | 309 |
| 1514754 | 3152 | 3167 | N/A | N/A | CCAAATGCTGTCCTAG | 17 | D | 310 |
| 1514764 | 1683 | 1698 | N/A | N/A | CCATAGCACTGAAATC | 42 | D | 311 |
| 1514768 | 1430 | 1445 | N/A | N/A | CCATTTATCTTCAGCG | 2 | D | 312 |
| 1514769 | 3984 | 3999 | 2067 | 2082 | ACACTAGGTAGACATC | 28 | D | 313 |
| 1514774 | 3272 | 3287 | 1568 | 1583 | TACCATACTCTGCCAG | 19 | D | 314 |
| 1514788 | 773 | 788 | 465 | 480 | TCAGAACCACTTCTGG | 47 | D | 315 |

### Example 3: Dose-Dependent Inhibition of Mouse COASY in 4T1 Cells by Modified Oligonucleotides

Modified oligonucleotides selected from the examples above were tested at various doses in 4T1 cells (described herein above). Cultured 4T1 cells at a density of 7,000 cells per well were treated by free uptake with various concentrations of modified oligonucleotide as specified in the tables below. After a treatment period of approximately 48 hours, total RNA was isolated from the cells, and COASY RNA levels were measured by quantitative real-time RTPCR. Mouse COASY primer-probe set RTS52828 (described herein above) was used to measure RNA levels as described above. COASY RNA levels were normalized to total RNA content, as measured by RIBOGREEN®. Reduction of COASY RNA is presented in the tables below as percent COASY RNA, relative to untreated control cells (% UTC).

The half maximal inhibitory concentration (IC₅₀) of each modified oligonucleotide was calculated using a linear regression on a log/linear plot of the data in Excel and is also presented in the tables below.

TABLE 4
Dose-dependent reduction of mouse COASY RNA
in 4T1 cells by modified oligonucleotides

| | COASY RNA (% UTC) | | | | IC₅₀ |
|---|---|---|---|---|---|
| Compound No. | 296 nM | 889 nM | 2667 nM | 8000 nM | (μM) |
| 1514501 | 30 | 27 | 16 | 7 | <0.3 |
| 1514503 | 11 | 6 | 4 | 2 | <0.3 |
| 1514505 | 42 | 35 | 25 | 18 | <0.3 |
| 1514529 | 58 | 41 | 30 | 20 | 0.51 |
| 1514542 | 82 | 66 | 42 | 35 | 2.31 |
| 1514555 | 28 | 19 | 11 | 7 | <0.3 |
| 1514558 | 48 | 41 | 32 | 25 | <0.3 |
| 1514568 | 67 | 49 | 41 | 23 | 1.05 |
| 1514569 | 61 | 47 | 30 | 16 | 0.66 |
| 1514573 | 40 | 37 | 23 | 15 | <0.3 |
| 1514584 | 41 | 32 | 23 | 15 | <0.3 |
| 1514592 | 54 | 42 | 27 | 14 | 0.43 |
| 1514614 | 63 | 60 | 48 | 25 | 1.42 |
| 1514625 | 47 | 41 | 29 | 18 | <0.3 |
| 1514678 | 26 | 18 | 9 | 4 | <0.3 |
| 1514702 | 59 | 48 | 30 | 19 | 0.65 |
| 1514737 | 56 | 46 | 35 | 19 | 0.57 |
| 1514768 | 17 | 9 | 5 | 3 | <0.3 |
| 1514772 | 55 | 43 | 33 | 22 | 0.48 |

TABLE 5
Dose-dependent reduction of mouse COASY RNA
in 4T1 cells by modified oligonucleotides

| | COASY RNA (% UTC) | | | | IC₅₀ |
|---|---|---|---|---|---|
| Compound No. | 296 nM | 889 nM | 2667 nM | 8000 nM | (μM) |
| 1514481 | 69 | 55 | 40 | 29 | 1.36 |
| 1514482 | 28 | 20 | 13 | 7 | <0.3 |
| 1514498 | 84 | 82 | 68 | 84 | >8.0 |
| 1514516 | 63 | 53 | 47 | 30 | 1.25 |
| 1514526 | 52 | 52 | 34 | 18 | 0.57 |
| 1514555 | 24 | 13 | 9 | 5 | <0.3 |
| 1514561 | 39 | 31 | 17 | 11 | <0.3 |
| 1514563 | 75 | 60 | 42 | 32 | 1.79 |
| 1514577 | 51 | 40 | 31 | 16 | 0.34 |
| 1514596 | 78 | 59 | 39 | 21 | 1.48 |
| 1514602 | 71 | 55 | 36 | 23 | 1.20 |
| 1514608 | 62 | 47 | 35 | 26 | 0.77 |
| 1514658 | 65 | 50 | 32 | 16 | 0.83 |
| 1514667 | 49 | 40 | 25 | 14 | <0.3 |
| 1514673 | 41 | 34 | 29 | 14 | <0.3 |
| 1514712 | 50 | 40 | 29 | 16 | 0.33 |
| 1514726 | 47 | 45 | 29 | 19 | <0.3 |
| 1514734 | 94 | 95 | 90 | 93 | >8.0 |
| 1514746 | 73 | 60 | 44 | 25 | 1.59 |

### Example 4: Design of Oligomeric Compounds Complementary to a Mouse COASY Nucleic Acid

Oligomeric compounds were designed as indicated in the tables below. Modified oligonucleotides described in the Examples above (parent compounds) were further modified by adding a THA-C6-GalNAc3 conjugate (designated as [THA-GalNAc] in the table below) at the 5′ end of the modified oligonucleotide. THA-GalNAc is represented by the structure below wherein the phosphate group is attached to the 5′-oxygen atom of the 5′ nucleoside:

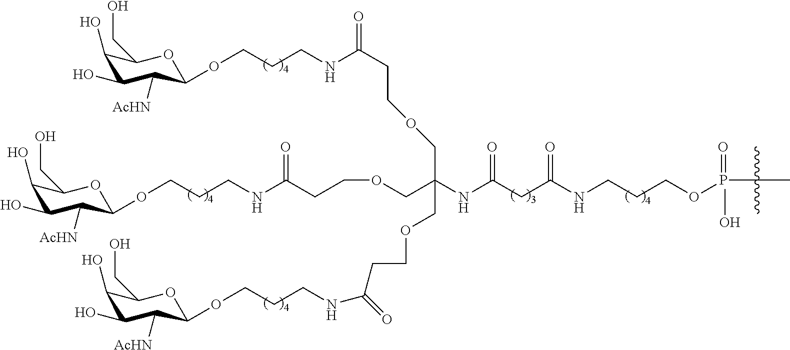

The chemistry notation column in the table below specifies the specific chemistry notation for modified oligonucleotides; wherein subscript ‘d’ represents a 2′-β-D-deoxyribosyl sugar moiety, subscript ‘k’ represents a cEt sugar moiety, subscript 's′ represents a phosphorothioate internucleoside linkage, and superscript ‘m’ before the cytosine residue (^{m}C) represents a 5-methylcytosine.

TABLE 6
Design of GalNAc conjugated modified oligonucleotides complementary to mouse COASY

| | Parent | | SEQ |
|---|---|---|---|
| Compound | Compound | | ID |
| No. | No. | Sequence and Chemistry notation (5′ to 3′) | No. |
| 1527085 | 1514673 | [THA-GalNAc]-AₖₛTₖₛ^{m}CₖₛT_{ds}T_{ds}T_{ds}A_{ds}A_{ds}T_{ds}T_{ds}^{m}C_{ds}A_{ds}G_{ds}TₖₛTₖₛAₖ | 138 |
| 1527089 | 1514503 | [THA-GalNAc]-GₖₛGₖₛTₖₛA_{ds}A_{ds}G_{ds}A_{ds}G_{ds}G_{ds}G_{ds}^{m}C_{ds}A_{ds}T_{ds}Tₖₛ^{m}CₖₛGₖ | 249 |

### Example 5: Effect of Modified Oligonucleotides Complementary to Mouse COASY Nucleic Acid in a Mouse DIO Model

Diet Induced Obesity (DIO) mice represent a model of Nonalcoholic Fatty Liver Disease (NAFLD). Male C57BL/6 mice (Jackson Laboratories) were put on a High Fat Diet (HFD) for 15 weeks (Research Diets Cat #D12492) to induce NAFLD. Groups of five HFD fed mice were then injected subcutaneously once a week for six weeks (a total of seven treatments) with 5 mg/kg of modified oligonucleotides. A group of three male HFD fed C57BL/6 mice was injected with PBS.

Compound No. 1287694, a control modified oligonucleotide with specific chemistry notation (from 5′ to 3′) of [THA-GalNAc]-^{m}CₖₛGₖₛ^{m}Cₖₛ^{m}C_{ds}G_{ds}A_{ds}T_{ds}A_{ds}A_{ds}G_{ds}G_{ds}T_{ds}A_{ds}mCₖₛAₖₛ^{m}Cₖ, wherein subscript ‘d’ represents a 2′-β-D-deoxyribosyl sugar moiety, subscript ‘k’ represents a cEt sugar moiety, subscript 's′ represents a phosphorothioate internucleoside linkage, and superscript ‘m’ before the cytosine residue (^{m}C) represents a 5-methylcytosine was designed to not target COASY.

### RNA Analysis

Mice were sacrificed on day 45, and RNA was extracted from liver tissue for quantitative real time RTPCR analysis of COASY RNA using mouse primer probe set RTS52828 (described herein above) COASY RNA levels were normalized to total RNA content, as measured by cyclophilin A. Mouse cyclophilin A was amplified using mouse primer probe set m cyclo24 (forward sequence TCGCCGCTTGCTGCA, designated herein as SEQ ID NO: 8; reverse sequence ATCGGCCGTGATGTCGA, designated herein as SEQ ID NO: 9; probe sequence CCATGGTCAACCCCACCGTGTTC, designated herein as SEQ ID NO: 10). Reduction of COASY RNA is presented in the table below as percent COASY RNA relative to the amount in liver tissue from PBS control animals (% control).

TABLE 7
Reduction of mouse COASY RNA in DIO Mice

| Compound No. | COASY RNA (% control) |
|---|---|
| PBS | 100 |
| 1287694 | 89.2 |
| 1527085 | 4.4 |
| 1527089 | 2.5‡ |

‡indicates that fewer than 5 samples were available

### Body and Organ Weights

Body weights of C57BL/6 mice were measured on days 1 and 45, and the average body weight for each group is presented in the table below. Liver, kidney, spleen, and fat pad weights were measured on the day the mice were sacrificed (day 45), and the average organ weights for each group are presented in the tables below.

TABLE 8
Body and organ weights (in grams)

| Compound | Body weight (g) | | Organ weight (g) | | | |
|---|---|---|---|---|---|---|
| No. | Day 1 | Day 45 | Liver | Kidney | Spleen | Fat Pad |
| PBS | 43 | 48 | 2.29 | 0.33 | 0.11 | 1.35 |
| 1287694 | 38 | 46 | 2.88 | 0.32 | 0.09 | 1.75 |
| 1527085 | 38 | 41 | 1.73 | 0.36 | 0.10 | 1.59 |
| 1527089 | 37 | 38‡ | 1.57‡ | 0.35‡ | 0.09‡ | 1.11‡ |

‡indicates that fewer than 5 samples were available

### Plasma Chemistry Markers

Plasma was collected when mice were sacrificed on day 45. To evaluate the effect of modified oligonucleotides on liver and kidney function, plasma levels of alanine aminotransferase (ALT), aspartate aminotransferase (AST), total bilirubin (TBIL), cholesterol (CHOL), glucose (GLUC), high-density lipoproteins (HDL), low-density lipoproteins (LDL), triglycerides (TRIG), and blood urea nitrogen (BUN) were measured using an automated clinical chemistry analyzer (Hitachi Olympus AU400c, Melville, NY). The results were averaged for each group of mice and are presented in the tables below.

Treatment of the DIO mice with modified oligonucleotides complementary to COASY nucleic acid resulted in decreases in ALT, AST, CHOL, glucose, and LDL relative to DIO mice that did not receive modified oligonucleotides complementary to COASY (PBS and control) as shown below.

TABLE 9
Plasma chemistry markers in DIO mice treated with modified
oligonucleotides complementary to mouse COASY nucleic acid

| Compound | ALT | AST | TBIL | CHOL | GLUC | HDL | LDL | TRIG | BUN |
|---|---|---|---|---|---|---|---|---|---|
| No. | (U/L) | (U/L) | (mg/dL) | (mg/dL) | (mg/dL) | (mg/dL) | (mg/dL) | (mg/dL) | (mg/dL) |
| PBS | 162 | 131 | 0.19 | 201 | 328 | 187 | 28 | 65 | 21 |
| 1287694 | 260 | 152 | 0.20 | 210 | 363 | 191 | 30 | 77 | 21 |
| 1527085 | 67 | 109 | 0.19 | 138 | 329 | 123 | 18 | 118 | 21 |
| 1527089 | 68‡ | 80‡ | 0.19‡ | 167‡ | 260‡ | 151‡ | 20‡ | 116‡ | 20‡ |

‡indicates that fewer than 5 samples were available

### Liver Triglycerides

Liver triglyceride levels were measured after the mice were sacrificed on day 45 using the Triglycerides Liquid Reagents from Pointe Scientific (Cat #T7532). The results were normalized to liver punch weights. Data is presented as liver TRIG (mg)/liver (g).

Treatment of DIO mice with COASY modified oligonucleotides led to a decrease in liver triglycerides compared to PBS treated controls.

TABLE 10
Liver triglycerides in DIO mice treated with modified oligonucleotides
complementary to mouse COASY nucleic acid

| | Liver Triglyceride |
|---|---|
| Compound No. | (mg)/liver (g) |
| PBS | 232 |
| 1287694 | 249 |
| 1527085 | 72 |
| 1527089 | 61‡ |

‡indicates that fewer than 5 samples were available

### Liver Steatosis

To evaluate the effect of modified oligonucleotides on steatosis, Oil Red O staining was carried out after the mice were sacrificed on day 45 to detect levels of neutral triglycerides and lipids. Levels of lipid accumulation in the liver were scored using Visiopharm Image Analysis software. Oil Red O stain levels are presented as a percentage of total liver area.

Treatment of DIO mice with modified oligonucleotides complementary to COASY nucleic acid resulted in a decrease in steatosis compared to PBS treated controls.

TABLE 11
Steatosis in DIO mice treated with modified oligonucleotides
complementary to mouse COASY nucleic acid

| | Oil Red O Stain |
|---|---|
| Compound No. | (% total liver area) |
| PBS | 24 |
| 1287694 | 26 |
| 1527085 | 7.5 |
| 1527089 | 0.07 |

Additionally, the degree of steatosis was determined by pathologist's blinded assessment of hematoxylin and eosin (H&E) stained formalin fixed, paraffin embedded liver sections. Steatosis scoring (0-5 where numbers reflect increased severity) was performed on sections and average scores were computed.

TABLE 11a
Steatosis in DIO mice treated with modified oligonucleotides
complementary to mouse COASY nucleic acid

| Compound No. | Steatosis score |
|---|---|
| PBS | 2.0 |
| 1287694 | 2.8 |
| 1527085 | 0.4 |
| 1527089 | 0.0 |

### Example 6: Effect of Modified Oligonucleotides Complementary to Mouse COASY Nucleic Acid in a GAN NASH Model

Gubra-Amylin NASH (GAN) diet-fed mice represent a model of Non-Alcoholic SteatoHepatitis (NASH). Groups of eight male C57BL/6 mice (Taconic) were fed a GAN diet rich in falt (40kca %)., fructose (20 kcal %1₁) and cholesterol (2 kcal %/1) for 31 weeks (Research Diets Cat #D09100310) to induce NASH. Groups of eight GAN diet-fed mice were injected subcutaneously once a week for twelve weeks (a total of thirteen treatments) with 5 mg/kg of a modified oligonucleotide complementary to COASY nucleic acid or a control modified oligonucleotide. One group of eight male GAN diet fed C57BL/6 mice was injected with PBS. The mice were compared to a group of 4 mice that were fed normal chow and left untreated. The mice were euthanized forty-eight hours post final treatment.

### RNA Analysis

Mice were sacrificed on Day 85, and RNA was extracted from liver tissue for quantitative real time RTPCR analysis of COASY RNA using mouse primer probe set RTS52828 (described herein above). COASY RNA levels are normalized to total RNA content, as measured by RIBOGREEN®.

TABLE 12
Reduction of mouse COASY RNA in mouse NASH model

| | COASY RNA |
|---|---|
| Compound No. | (% control) |
| PBS | 100 |
| 1287694 | 102 |
| 1527085 | 3‡ |
| 1527089 | 3‡ |
| normal chow | 98 |

‡indicates that fewer than 8 samples were available

### Body and Organ Weights

Body weights of GAN-fed C57BL/6 mice were measured on day 85, and the average body weight for each group is presented in the table below. Liver, kidney, spleen, and epidydimal white adipose tissue (WAT) weights were measured on the day the mice were sacrificed (day 85), and the average organ weights for each group are presented in the tables below.

TABLE 13
Body and organ weights (in grams)

| | Body weight (g) | Organ weight (g) | | | |
|---|---|---|---|---|---|
| Compound No. | on Day 85 | Liver | Kidney | Spleen | WAT |
| PBS | 53 | 4.7 | 0.4 | 0.1 | 2.3 |
| 1287694 | 51 | 4.6 | 0.4 | 0.1 | 2.1 |
| 1527085 | 52 | 4.0 | 0.4 | 0.1 | 2.7 |
| 1527089 | 49 | 3.8 | 0.4 | 0.1 | 2.2 |
| Normal Chow | 40 | 1.6 | 0.4 | 0.1 | 1.7 |

### Plasma Chemistry Markers

Plasma was collected when mice were sacrificed on Day 85. To evaluate the effect of modified oligonucleotides on liver and kidney function, plasma levels of alanine aminotransferase (ALT), aspartate aminotransferase (AST), total bilirubin (TBIL), cholesterol (CHOL), high-density lipoproteins (HDL), low-density lipoproteins (LDL), triglycerides (TRIG), glucose (GLUC), and blood urea nitrogen (BUN) were measured using an automated clinical chemistry analyzer (Hitachi Olympus AU400c, Melville, NY).

Treatment of NASH model with modified oligonucleotides complementary to COASY resulted in decreases in several plasma chemistry markers as shown below.

TABLE 14
Plasma chemistry markers in mouse NASH model treated with
modified oligonucleotides complementary to mouse COASY

| Compound | ALT | AST | TBIL | CHOL | HDL | LDL | TRIG | BUN | GLUC |
|---|---|---|---|---|---|---|---|---|---|
| No. | (U/L) | (U/L) | (mg/dL) | (mg/dL) | (mg/dL) | (mg/dL) | (mg/dL) | (mg/dL) | (mg/dL) |
| PBS | 601 | 443 | 0.24 | 431 | 253 | 149 | 66 | 18 | 279 |
| 1287694 | 754 | 458 | 0.24 | 438 | 244 | 166 | 74 | 18 | 257 |
| 1527085 | 317 | 269 | 0.18 | 303 | 203 | 85 | 66 | 17 | 265 |
| 1527089‡ | 146 | 164 | 0.17 | 344 | 221 | 78 | 56 | 16 | 224 |
| normal chow | 36 | 52 | 0.13 | 114 | 94 | 14 | 157 | 18 | 296 |

‡indicates that fewer than 8 samples were available

### Liver Triglycerides

Liver triglyceride levels were measured using the Triglycerides Liquid Reagents from Pointe Scientific (Cat#T7532). The results were normalized to liver punch weights. Treatment of a NASH model with modified oligonucleotides complementary to COASY nucleic acid results in a decrease in liver triglyceride levels compared to PBS treated controls.

TABLE 15
Liver triglyceride in mouse NASH model treated with modified
oligonucleotides complementary to mouse COASY nucleic acid

| | Liver Triglyceride |
|---|---|
| Compound No. | (mg)/liver (g) |
| PBS | 150 |
| 1287694 | 153 |
| 1527085 | 131 |
| 1527089‡ | 57 |
| normal chow | 52 |

‡indicates that fewer than 8 samples were available

### Fibrosis Markers

To evaluate the effect of modified oligonucleotides on fibrosis, liver levels of hydroxyproline were measured. Liver hydroxyproline was quantified using the QuickZyme hydroxyproline kit (QuickZyme Biosciences, Cat. #QZBHYPRO5). The results were normalized to total protein levels measured using QuickZyme Biosciences total protein assay kit (Cat. #QZBTOTPROT5).

Liver levels of collagen were quantified using Picro-Sirius Red staining and scored using Visiophal1 Image Analysis software. PSR stain levels are presented as a percentage of total liver area.

Additionally, liver levels of Collal were quantified histologically using IHC staining with LSBio antibody LS-C343921-100, and scored using Visiopharm Image Analysis software. The Collal levels are presented as a percentage of total liver area.

TABLE 16
Fibrosis markers in mouse NASH model treated with modified
oligonucleotides complementary to mouse COASY nucleic acid

| | Hydroxyproline | Picosirius Red Stain | Col1a1 |
|---|---|---|---|
| | (μM)/Total | (% total | (% total |
| Compound No. | Protein (mg/ml) | liver area) | liver area) |
| PBS | 6 | 3.8 | 6.7 |
| 1287694 | 5 | 3.4 | 5.9 |
| 1527085 | 8 | 4.3 | 6.3‡ |
| 1527089 | 5‡ | 4.4 | 5.6 |
| normal chow | 2 | 0.5 | 0.4 |

‡indicates that fewer than 8 samples were available

To further evaluate the effect of modified oligonucleotides on fibrosis, RNA levels of markers of fibrosis and inflammation such as α-SMA, COLlA1, TIMP1, TNFα, and TGFβ1 were measured using quantitative real-time RTPCR. The primer-probe sets used to measure RNA levels of mouse α-SMA, COL1A1, TIMP1, and TGFβ1 are listed in the table below.

TABLE 17
List of mouse primer-probe sets used for RNA analysis

| | primer- | | SEQ | | SEQ | | SEQ |
|---|---|---|---|---|---|---|---|
| Target | probe set | Forward | ID | Reverse | ID | | ID |
| RNA | name | primer | NO | Primer | NO | Probe | NO |
| α-SMA | mActa2_ | TGCCTCTAGCAC | 316 | GCAGGAATGAT | 317 | CGTTTTGTGGAT | 318 |
| | LTS00192 | ACAACTGTGA | | TTGGAAAGGAA | | CAGCGCCTCCA | |
| COL1A1 | mcol1a1 | TGGATTCCCGTT | 319 | TCAGCTGGATA | 320 | AAGCGAGGGCT | 321 |
| | | CGAGTACG | | GCGACATC | | CCGACCCGA | |
| TIMP1 | LTS00190 | TCATGGAAAGC | 322 | GCGGCCCGTGA | 323 | CCCACAAGTCCC | 324 |
| | | CTCTGTGGAT | | TGAGA | | AGAACCGCAGT | |
| | | | | | | G | |
| TGFß1 | mTGFb1_ | AAACGGAAGCG | 325 | GGGACTGGCGA | 326 | CCATCCGTGGCC | 327 |
| | 12113 | CATCGAA | | GCCTTAGTT | | AGATCCTGTCC | |
| TNFα | RTS2501 | CAGGTTCTGTCC | 328 | CTGTGCTCATGG | 329 | CCCAAGGCGCC | 330 |
| | | CTTTCACTCACT | | TGTCTTTTCTG | | ACATCTCCCT | |

The levels of α-SMA RNA expression are averaged for each group of mice and are presented as percent α-SMA RNA, relative to the amount in PBS treated animals, normalized to total RNA content, as measured by RIBOGREEN® (% control).

The levels of COL1A1 RNA expression are averaged for each group of mice and are presented as percent COL1A1 RNA, relative to the amount in PBS treated animals, normalized to total RNA content, as measured by RIBOGREEN® (% control).

The levels of TIMP1 RNA expression are averaged for each group of mice and are presented as percent TIMP1 RNA, relative to the amount in PBS treated animals, normalized to total RNA content, as measured by RIBOGREEN® (% control).

The levels of TGF$1 RNA expression are averaged for each group of mice and are presented as percent TGF$1 RNA, relative to the amount in PBS treated animals, normalized to total RNA content, as measured by RIBOGREEN® (% control).

Treatment of a NASH model with modified oligonucleotides complementary to COASY nucleic acid results in a decrease in liver fibrosis and inflammation markers compared to PBS treated controls.

TABLE 18
Effect of modified oligonucleotides complementary
to mouse COASY on RNA expression of fibrosis
and inflammation markers in a NASH model

| Compound | RNA (% control) | | | | | |
|---|---|---|---|---|---|---|
| No. | COASY | α-SMA | Col1A1 | TIMP1 | TNFα | TGFβ1 |
| PBS | 100 | 100 | 100 | 100 | 100 | 100 |
| 1287694 | 102 | 90 | 110 | 111 | 126 | 120 |
| 1527085‡ | 4 | 57 | 56 | 57 | 86 | 93 |
| 1527089‡ | 2 | 62 | 40 | 30 | 70 | 81 |
| normal | 98 | 35 | 3 | 3 | 28 | 35 |
| chow | | | | | | |

‡indicates that fewer than 8 samples were available

### Liver Steatosis

To evaluate the effect of modified oligonucleotides on steatosis, oil red O staining was carried out after the mice were sacrificed on day 85 to detect levels of neutral triglycerides and lipids. Levels of lipid accumulation in the liver were scored using Visiopharm Image Analysis software. Oil Red O stain levels are presented as a percentage of total liver area.

Treatment of a NASH model with a modified oligonucleotide complementary to COASY nucleic acid resulted in a decrease in steatosis compared to PBS treated controls.

TABLE 19
Steatosis in mouse NASH model treated with modified oligonucleotides
complementary to mouse COASY nucleic acid

| | Oil Red O |
|---|---|
| | (% total |
| Compound No. | liver area) |
| PBS | 39 |
| 1287694 | 44 |
| 1527085 | 38 |
| 1527089 | 17 |
| normal chow | 12 |

### Example 7: Design of Modified Oligonucleotides Complementary to a Mouse COASY Nucleic Acid

Modified oligonucleotides were designed as indicated in the table below. Modified oligonucleotides described in the Examples above (parent compounds) were further modified by adding a THA-C6-GalNAc3 conjugate (designated as [THA-GalNAc] in the table below) at the 5′ end of the modified oligonucleotide. THA-GalNAc is represented by the structure below wherein the phosphate group is attached to the 5′-oxygen atom of the 5′ nucleoside:

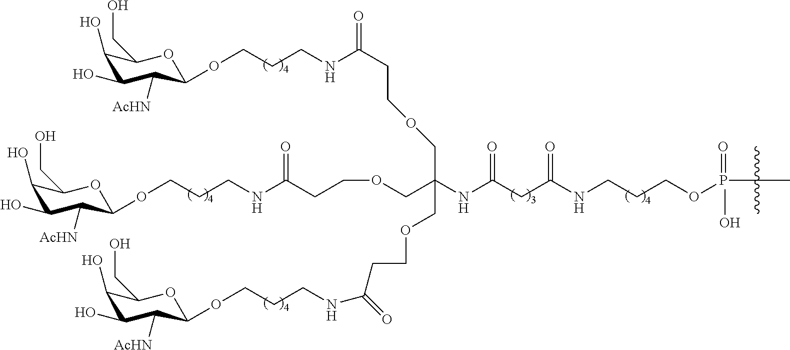

The chemistry notation column in the table below specifies the specific chemistry notation for modified oligonucleotides; wherein subscript ‘d’ represents a 2′-β-D-deoxyribosyl sugar moiety, subscript ‘k’ represents a cEt sugar moiety, subscript 's′ represents a phosphorothioate internucleoside linkage, and superscript ‘m’ before the cytosine residue (^{m}C) represents a 5-methylcytosine.

TABLE 20
Design of GalNAc conjugated modified oligonucleotides complementary to mouse COASY

| Conjugated | Parent | | SEQ |
|---|---|---|---|
| Compound | Compound | | ID |
| No. | No. | Sequence and Chemistry notation (5′ to 3′) | No. |
| 1527084 | 1514667 | [THA-GalNAc]-^{m}Cₖₛ^{m}CₖₛAₖₛA_{ds}T_{ds}A_{ds}G_{ds}G_{ds}T_{ds}A_{ds}T_{ds}^{m}C_{ds}A_{ds}GₖₛTₖₛTₖ | 207 |
| 1527086 | 1514768 | [THA-GalNAc]-^{m}Cₖₛ^{m}CₖₛAₖₛT_{ds}T_{ds}T_{ds}A_{ds}T_{ds}^{m}C_{ds}T_{ds}T_{ds}^{m}C_{ds}A_{ds}Gₖₛ^{m}CₖₛGₖ | 312 |
| 1527091 | 1514678 | [THA-GalNAc]-AₖₛAₖₛTₖₛA_{ds}G_{ds}G_{ds}T_{ds}A_{ds}T_{ds}^{m}C_{ds}A_{ds}G_{ds}T_{ds}TₖₛGₖₛGₖ | 290 |

### Example 8: Effects of Modified Oligonucleotides Complementary to Mouse COASY in Wildtype Mice, Multiple Dose

Wild type C57BL/B mice (Jackson Laboratory) were treated with modified oligonucleotides described above to determine activity of modified oligonucleotides complementary to mouse COASY.

### Treatment

Groups of four male C57BL/6 mice were administered a single subcutaneous injection of modified oligonucleotides at doses indicated in the tables below. One group of four male C57BL/6 mice was injected with PBS. The PBS-injected group served as the control group to which modified oligonucleotide-treated groups were compared.

Compound No. 1287694 (described herein above) was added as a control.

### RNA Analysis

72 hours post treatment, the mice were sacrificed, and RNA was extracted from liver tissue for quantitative real time RTPCR analysis of COASY RNA expression. Mouse primer probe set RTS52828 (described herein above) was used to measure mouse COASY RNA levels. COASY RNA levels were normalized to total RNA content, as measured by RIBOGREEN®. Results are presented as percent mouse COASY RNA, relative to the amount of mouse COASY RNA in PBS treated animals (% control). ED50s were calculated in Prism using nonlinear fit with variable slope (three parameter), top constrained to 100% (or 1), bottom constrained to 0. Y=Bottom+(Top-Bottom)/(1+(IC50/X)\hat{ }HillSlope).

TABLE 21
Reduction of mouse COASY RNA in wildtype mice

Liver

| | Dose | COASY RNA | ED50 |
|---|---|---|---|
| Compound No. | (mpk) | (% control) | (mpk) |
| PBS | 0 | 100 | — |
| 1287694 | 18 | 85 | — |
| 1527085 | 18 | 5 | 0.69 |
| | 6 | 15 | |
| | 2 | 27 | |
| 1527089 | 18 | 9 | 0.66 |
| | 6 | 20 | |
| | 2 | 30 | |
| 1527084 | 18 | 7 | 3.07 |
| | 6 | 38 | |
| | 2 | 53 | |
| 1527086 | 18 | 9 | 1.33 |
| | 6 | 27 | |
| | 2 | 41 | |
| 1527091 | 18 | 2 | 1.98 |
| | 6 | 10 | |
| | 2 | 43 | |

### Example 9: Design of RNAi Agents Targeted to Mouse COASY Nucleic Acid

Double-stranded siRNA (siRNA) comprising antisense oligonucleotides complementary to mouse COASY nucleic acid, and sense oligonucleotides complementary to the antisense oligonucleotides are designed as follows.

Each antisense oligonucleotide is complementary to the target mouse COASY nucleic acid (SEQ ID NO: 1 (ENSEMBL Accession No. ENSMUSG00000001755.12 from version 102: November 2020). Each antisense oligonucleotide may comprise at least 12, at least 13, at least 14, at least 15, or 16 contiguous nucleobases of the nucleobase sequence of any of SEQ ID NOs: 15-315.

The antisense oligonucleotide in each case is 23 nucleosides in length; has a sugar motif (from 5′ to 3′) of: yfyfyfyfyfyfyfyfyfyfyyy; wherein each ‘y’ represents a 2′-OMe sugar moiety and each “f” represents a 2′-F sugar moiety; and an internucleoside linkage motif (from 5′ to 3′) of: ssooooooooooooooooooss; wherein ‘o’ represents a phosphodiester internucleoside linkage and 's′ represents a phosphorothioate internucleoside linkage. Each cytosine residue is a non-methylated cytosine. Each antisense oligonucleotide has a terminal phosphate at the 5′-end.

Each sense oligonucleotide is complementary to the first of the 21 nucleosides of the antisense oligonucleotide (from 5′ to 3′) wherein the last two 3′-nucleosides of the antisense oligonucleotides are not paired with the sense oligonucleotide (are overhanging nucleosides). Each sense oligomeric compound may further contain a GalNAc moiety conjugated to the 3′-oxygen as shown below:

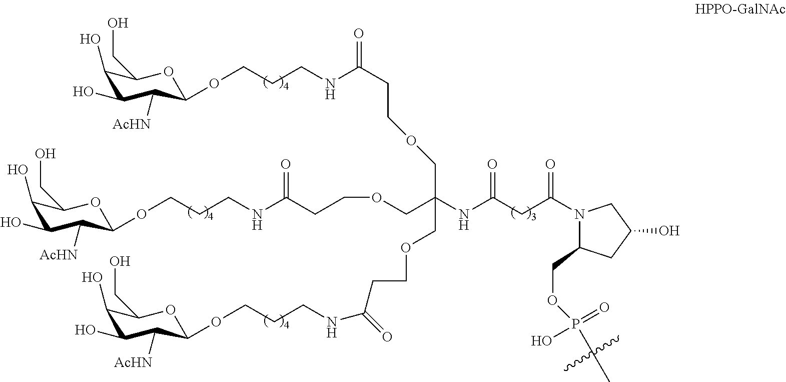

The sense oligonucleotide in each case is 21 nucleosides in length; has a sugar motif (from 5′ to 3′) of: fyfyfyfyfyfyfyfyfyfyf; wherein each ‘y’ represents a ribo-2′-OMe sugar moiety and each “f” represents a 2′-F sugar moiety; and an internucleoside linkage motif (from 5′ to 3′) of: ssooooooooooooooooss; wherein ‘o’ represents a phosphodiester internucleoside linkage and 's′ represents a phosphorothioate internucleoside linkage.

Single-stranded RNAi (ssRNAi) RNAi agents comprising antisense oligonucleotides complementary to mouse COASY nucleic acid are designed as follows.

The antisense oligonucleotide in each case is 23 nucleosides in length; has a sugar motif (from 5′ to 3′) of: yfyfyfyfyfyfyfyfyfyfyyy; wherein each ‘y’ represents a ribo-2′-OMe sugar moiety and each “f” represents a 2′-F sugar moiety; and an internucleoside linkage motif (from 5′ to 3′) of: ssooooooooooooooooooss; wherein ‘o’ represents a phosphodiester internucleoside linkage and 's′ represents a phosphorothioate internucleoside linkage. Each cytosine residue is a non-methylated cytosine. Each antisense oligonucleotide has a terminal phosphate at the 5′-end.

Each antisense oligonucleotide is complementary to the target mouse COASY nucleic acid (SEQ ID NO: 1 (ENSEMBL Accession No. ENSMUSG00000001755.12 from version 102: November 2020)). Each antisense oligonucleotide may comprise at least 12, at least 13, at least 14, at least 15, or 16 contiguous nucleobases of the nucleobase sequence of any of SEQ ID NOs: 15-315.

### Example 10: Effect of RNAi Agents Targeted to Mouse COASY Nucleic Acid on Mouse COASY RNA In Vitro, Single dose

Cultured A431 cells or mouse primary hepatocytes are treated with RNAi agents designed according to Example 9 at a concentration of 0.1-20 nM by RNAiMAX at a density of 20,000 cells per well. After a treatment period of approximately 24 hours, total RNA is isolated from the cells and COASY RNA levels are measured by quantitative real-time RTPCR. COASY RNA levels are measured by mouse primer-probe set RTS52828 (described herein above). COASY RNA levels are normalized to total RNA content, as measured by RIBOGREEN®. Reduction of COASY RNA is assessed as percent COASY RNA relative to the amount of COASY RNA in untreated control cells (% UTC).

### Example 11: Effect of RNAi Agents Targeted to Mouse COASY Nucleic Acid in a GAN NASH Model and/or a DIO NAFLD Model Gubra-Amylin NASH (GAN) diet-fed mice represent a model of Non-Alcoholic SteatoHepatitis (NASH)

Groups of male C57BL/6 mice (Taconic) are fed a GAN diet rich in fat (40 kcal %), fructose (20 kcal %) and cholesterol (2 kcal %) for 31 weeks (Research Diets Cat #D09100310) to induce NASH.

Diet Induced Obesity (DIO) mice represent a model of Nonalcoholic Fatty Liver Disease (NAFLD). Male C57BL/6 mice (Jackson Laboratories) are put on a High Fat Diet (HFD) for 15 weeks (Research Diets Cat #D12492) to induce NAFLD.

Groups of GAN diet-fed mice and/or DIO mice receive a single subcutaneous injection of an RNAi agent designed according to Example 9 at a dose of 1 mg/kg. One group of GAN diet fed mice and/or DIO mice is injected with PBS. The mice are euthanized one week post treatment.

RNA expression of mouse COASY, plasma chemistry markers, liver triglyceride levels, liver steatosis levels, and/or liver fibrosis levels are measured in the euthanized RNAi agent-treated DIO mice and PBS-control mice (see example 5 herein above).

RNA expression of mouse COASY, plasma chemistry markers, liver triglyceride levels, liver steatosis levels, and/or liver fibrosis levels are measured in the euthanized RNAi agent-treated GAN diet-fed mice and PBS-control mice (see example 6 herein above).

Treatment of DIO mice or GAN diet-fed mice with RNAi agents that target COASY nucleic acid results in a reduction of COASY RNA levels, a decrease in ALT, AST, CHOL, glucose, or LDL, a decrease in liver triglyceride levels, a decrease in steatosis, or a decrease in liver fibrosis, compared to PBS controls.

What is claimed is:

1. A method of treating a liver disease or disorder in a subject having a liver disease or disorder, comprising administering a COASY-specific inhibitor to the subject, thereby treating the liver disease or disorder in the subject.

2. The method of claim 1, wherein the liver disease or disorder is fatty liver disease, chronic liver disease, liver cirrhosis, hepatic steatosis, steatohepatitis, nonalcoholic fatty liver disease (NAFLD), alcoholic steatohepatitis (ASH), or nonalcoholic steatohepatitis (NASH).

3. A method comprising administering a COASY-specific inhibitor to a subject.

4. The method of claim 3, wherein the subject has a liver disease or is at risk for developing a liver disease.

5. The method of claim 4, wherein the the liver disease or disorder is fatty liver disease, chronic liver disease, liver cirrhosis, hepatic steatosis, steatohepatitis, NAFLD, ASH, or NASH.

6. The method of any of claims 1-5, wherein a therapeutic amount of the COASY-specific inhibitor is administered to the subject.

7. The method of any of claims 1-6, wherein a therapeutic amount of the COASY-specific inhibitor ameliorates at least one symptom of the liver disease.

8. The method of any of claims 1-7, wherein the administration of the COASY-specific inhibitor ameliorates at least one symptom of fatty liver disease, chronic liver disease, liver cirrhosis, hepatic steatosis, steatohepatitis, NAFLD, ASH, or NASH.

9. The method of claim 8, wherein the at least one symptom is hepatic steatosis, liver fibrosis, elevated triglyceride level, elevated plasma lipid level, elevated hepatic lipid level, elevated ALT level, high NAFLD Activity score, or elevated plasma cholesterol level.

10. The method of any of claims 1-9, wherein administering the COASY-specific inhibitor reduces hepatic steatosis, reduces liver fibrosis, reduces plasma lipid levels, reduces plasma triglyceride levels, reduces plasma cholesterol levels, reduces ALT levels, improves NAS, reduces hepatic lipid levels, reduces hepatic triglyceride levels, or reduces hepatic cholesterol levels in the subject, or a combination thereof.

11. The method of any of claims 1-10, wherein the COASY-specific inhibitor reduces levels of hydroxyproline, reduces levels of Collal, reduces levels of ORO, or reduces levels total collagen in the liver of the subject, or a combination thereof.

12. The method of any of claims 1-11, wherein the subject is a human subject.

13. A method comprising contacting a cell with a COASY-specific inhibitor.

14. The method of claim 13, wherein expression of COASY in the cell is reduced.

15. A method of inhibiting expression or activity of COASY in a cell comprising contacting the cell with a COASY-specific inhibitor, thereby inhibiting expression or activity of COASY in the cell.

16. The method of any of claims 13-15, wherein the cell is a hepatocyte.

17. The method of any of claims 13-16, wherein the cell is in a subject.

18. The method of claim 17, wherein the subject has, or is at risk of having liver disease, fatty liver disease, chronic liver disease, liver cirrhosis, hepatic steatosis, steatohepatitis, NAFLD, ASH, or NASH.

19. The method of any of claims 1-8, wherein the COASY-specific inhibitor is an antisense agent, a polypeptide, an antibody, or a small molecule.

20. The method of any of claims 1-19, wherein the COASY-specific inhibitor is an antisense agent comprising a modified oligonucleotide, wherein the modified oligonucleotide has a nucleobase sequence complementary to the nucleobase sequence of a COASY nucleic acid.

21. The method of any of claims 1-20, wherein the nucleobase sequence of the modified oligonucleotide is complementary to any of SEQ ID NOs: 1-4.

22. The method of claim 21, wherein the nucleobase sequence modified oligonucleotide is complementary to SEQ ID NO: 3 or SEQ ID NO: 4.

23. The method of claim 22, wherein the nucleobase sequence of the modified oligonucleotide is at least 90% complementary to an equal length portion of SEQ ID NO: 3 or SEQ ID NO: 4.

24. The method of claim 22, wherein the nucleobase sequence of the modified oligonucleotide is at least 95% complementary to an equal length portion of SEQ ID NO: 3 or SEQ ID NO: 4.

25. The method of claim 22, wherein the nucleobase sequence of the modified oligonucleotide is 100% complementary to an equal length portion of SEQ ID NO: 3 or SEQ ID NO: 4.

26. The method of any of claims 20-25, wherein at least one nucleoside of the modified oligonucleotide comprises a modified sugar moiety.

27. The method of claim 26, wherein the modified sugar moiety comprises a bicyclic sugar moiety.

28. The method of claim 27, wherein the bicyclic sugar moiety comprises a 4′—CH(CH₃)—O-2′ bridge or a 4′—(CH₂)ₙ—O-2′ bridge, wherein n is 1 or 2.

29. The method of claim 26, wherein the modified sugar moiety comprises a non-bicyclic modified sugar moiety.

30. The method of claim 29, wherein the non-bicyclic sugar moiety is a 2′-F, 2′-OMe, or 2′-MOE sugar moiety.

31. The method of any of claims 20-30, wherein the antisense agent is single-stranded.

32. The method of any of claims 20-30, wherein the antisense agent is double-stranded.

33. The method of any of claims 20-32, wherein the modified oligonucleotide consists of 12 to 30 linked nucleosides.

34. The method of any of claims 20-33, wherein at least one nucleoside of the modified oligonucleotide comprises a modified nucleobase.

35. The method of claim 34, wherein the modified nucleobase is 5-methylcytosine.

36. The method of any of claims 20-35, wherein at least one internucleoside linkage of the modified oligonucleotide is a modified internucleoside linkage.

37. The method of claim 36, wherein the at least one modified internucleoside linkage is a phosphorothioate internucleoside linkage.

38. The method of claim 36, wherein each internucleoside linkage is a phosphorothioate internucleoside linkage.

39. The method of claim 36, wherein each internucleoside linkage is independently selected from a phosphodiester internucleoside linkage and a phosphorothioate internucleoside linkage.

40. The method of any one of claims 20-39, wherein the modified oligonucleotide has:
a gap segment consisting of linked 2′-deoxynucleosides;
a 5′ wing segment consisting of linked nucleosides;
a 3′ wing segment consisting linked nucleosides;
wherein the gap segment is positioned immediately adjacent to and between the 5′ wing segment and the 3′ wing segment and wherein each nucleoside of each wing segment comprises a modified sugar.

41. The method of claim 40 wherein the gap segment is positioned immediately adjacent to and between the 5′ wing segment and the 3′ wing segment and wherein each nucleoside of each wing segment comprises a modified sugar moiety.

42. The method of any of claims 20-41, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides;
wherein the 3′-most nucleoside of the 5′-region and the 5′-most nucleoside of the 3′-region comprise modified sugar moieties, and
each of the central region nucleosides is selected from a nucleoside comprising a 2′-β-D-deoxyribosyl sugar moiety and a nucleoside comprising a 2′-substituted sugar moiety, wherein the central region comprises at least six nucleosides comprising a 2′-β-D-deoxyribosyl sugar moiety and no more than two nucleosides comprise a 2′-substituted sugar moiety.

43. The method of any of claim 1-42, wherein the COASY-specific inhibitor is administered parenterally.

44. The method of claim 43, wherein the COASY-specific inhibitor is administered parenterally by subcutaneous or intravenous administration.

45. The method of any of claims 1-44, comprising co-administering the COASY-specific inhibitor and at least one additional therapy.

46. The method of any of claims 20-45, wherein the antisense agent comprises a conjugate group.

47. The method of claim 46, wherein the conjugate group comprises N-acetyl galactosamine.

48. The method of any of claims 1-47, wherein the COASY-specific inhibitor is an RNase H agent capable of reducing the amount of COASY nucleic acid through the activation of RNase H.

49. The method of any of claims 1-47, wherein the COASY-specific inhibitor is an RNAi agent capable of reducing the amount of COASY nucleic acid through the activation of RISC/Ago2.

50. The method of any of claims 1-47, wherein the COASY-specific inhibitor is a steric-blocking agent capable of directly binding to a target nucleic acid, thereby blocking the interaction of the COASY nucleic acid with other nucleic acids or proteins.

51. Use of a COASY-specific inhibitor for the manufacture or preparation of a medicament for treating a liver disease or disorder.

52. Use of a COASY-specific inhibitor for the treatment of a liver disease or disorder.

53. The use of claim 51 or 52, wherein the liver disease or disorder is fatty liver disease, chronic liver disease, liver cirrhosis, hepatic steatosis, steatohepatitis, NAFLD, ASH, or NASH.

54. The use of any of claims 51-53, wherein the COASY-specific inhibitor reduces or improves hepatic steatosis, liver fibrosis, plasma lipid levels, plasma triglyceride levels, plasma cholesterol levels, ALT levels, NAFLD Activity Score (NAS), hepatic lipid levels, hepatic triglyceride levels, or hepatic cholesterol levels, or a combination thereof.

55. The use of any of claim 51-54, wherein the COASY-specific inhibitor reduces hepatic steatosis, reduces liver fibrosis, reduces plasma lipid levels, reduces plasma triglyceride levels, reduces plasma cholesterol levels, reduces ALT levels, improves NAS, reduces hepatic lipid levels, reduces hepatic triglyceride levels, or reduces hepatic cholesterol levels, or a combination thereof.

56. The use of any of claims 51-55, wherein the COASY-specific inhibitor reduces levels of hydroxyproline, reduces levels of Col1a1, reduces levels of ORO, or reduces levels total collagen in the liver, or a combination thereof.

57. The use of any of claims 51-56, wherein the COASY-specific inhibitor is an antisense agent, a polypeptide, an antibody, or a small molecule.

58. The use of any of claims 51-57, wherein the COASY-specific inhibitor is an antisense agent comprising a modified oligonucleotide, wherein the modified oligonucleotide has a nucleobase sequence complementary to the nucleobase sequence of a COASY nucleic acid.

59. The use of claim 58, wherein the nucleobase sequence of the modified oligonucleotide is complementary to any of SEQ ID NOs: 1-4.

60. The use of claim 58, wherein the nucleobase sequence modified oligonucleotide is complementary to SEQ ID NO: 3 or SEQ ID NO: 4.

61. The use of claim 58, wherein the nucleobase sequence of the modified oligonucleotide is at least 90% complementary to an equal length portion of SEQ ID NO: 3 or SEQ ID NO: 4.

62. The use of claim 58, wherein the nucleobase sequence of the modified oligonucleotide is at least 95% complementary to an equal length portion of SEQ ID NO: 3 or SEQ ID NO: 4.

63. The use of claim 58, wherein the nucleobase sequence of the modified oligonucleotide is 100% complementary to an equal length portion of SEQ ID NO: 3 or SEQ ID NO: 4.

64. The use of any of claims 58-63, wherein at least one nucleoside of the modified oligonucleotide comprises a modified sugar moiety.

65. The use of claim 64, wherein the modified sugar moiety comprises a bicyclic sugar moiety.

66. The use of claim 65, wherein the bicyclic sugar moiety comprises a 4′—CH(CH₃)—O-2′ bridge or a 4′-(CH₂)ₙ—O-2′ bridge, wherein n is 1 or 2.

67. The use of claim 64, wherein the modified sugar moiety comprises a non-bicyclic modified sugar moiety.

68. The use of claim 67, wherein the non-bicyclic sugar moiety is a 2′-F, 2′-OMe, or 2′-MOE sugar moiety.

69. The use of any of claims 58-68, wherein the antisense agent is single-stranded.

70. The use of any of claims 58-68, wherein the antisense agent is double-stranded.

71. The use of any of claims 58-70, wherein the modified oligonucleotide consists of 12 to 30 linked nucleosides.

72. The use of any of claims 58-71, wherein at least one nucleoside of the modified oligonucleotide comprises a modified nucleobase.

73. The use of claim 72, wherein the modified nucleobase is 5-methylcytosine.

74. The use of any of claims 58-73, wherein at least one internucleoside linkage of the modified oligonucleotide is a modified internucleoside linkage.

75. The use of claim 74, wherein the at least one modified internucleoside linkage is a phosphorothioate internucleoside linkage.

76. The use of claim 74, wherein each internucleoside linkage is a phosphorothioate internucleoside linkage.

77. The use of claim 74, wherein each internucleoside linkage is independently selected from a phosphodiester internucleoside linkage and a phosphorothioate internucleoside linkage.

78. The use of any one of claims 58-77, wherein the modified oligonucleotide has:
a gap segment consisting of linked 2′-deoxynucleosides;
a 5′ wing segment consisting of linked nucleosides;
a 3′ wing segment consisting linked nucleosides;
wherein the gap segment is positioned immediately adjacent to and between the 5′ wing segment and the 3′ wing segment and wherein each nucleoside of each wing segment comprises a modified sugar moiety.

79. The use of claim 78 wherein the gap segment is positioned immediately adjacent to and between the 5′ wing segment and the 3′ wing segment and wherein each nucleoside of each wing segment comprises a modified sugar moiety.

80. The use of any of claims 58-79, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides;
wherein the 3′-most nucleoside of the 5′-region and the 5′-most nucleoside of the 3′-region comprise modified sugar moieties, and
each of the central region nucleosides is selected from a nucleoside comprising a 2′-β-D-deoxyribosyl sugar moiety and a nucleoside comprising a 2′-substituted sugar moiety, wherein the central region comprises at least six nucleosides comprising a 2′-β-D-deoxyribosyl sugar moiety and no more than two nucleosides comprise a 2′-substituted sugar moiety.

81. The use of any of claim 51-81, wherein the COASY-specific inhibitor is administered parenterally.

82. The use of claim 81, wherein the COASY-specific inhibitor is administered parenterally by subcutaneous or intravenous administration.

83. The use of any of claims 51-82, comprising co-administering the COASY-specific inhibitor and at least one additional therapy.

84. The use of any of claims 58-83, wherein the antisense agent comprises a conjugate group.

85. The use of claim 84, wherein the conjugate group comprises N-acetyl galactosamine.

86. The use of any of claims 51-85, wherein the COASY-specific inhibitor is an RNase H agent capable of reducing the amount of COASY nucleic acid through the activation of RNase H.

87. The use of any of claims 51-85, wherein the COASY-specific inhibitor is an RNAi agent capable of reducing the amount of COASY nucleic acid through the activation of RISC/Ago2.

88. The use of any of claims 51-85, wherein the COASY-specific inhibitor is a steric-blocking agent capable of directly binding to a target nucleic acid, thereby blocking the interaction of the COASY nucleic acid with other nucleic acids or proteins.


SEQUENCE LISTING (331 sequences)

SEQ ID NO: 1 - DNA, 4055 bp, Mus musculus
gcctcgccccgccccaggagtctctcggcgatgctttgtttgattgctctgagttcccgtagctccacgctttgaggctggtgggtagtgcttccgggctgcagcctaggacgcctccacggcggtgtaagtagaagcagaatcccaagaactcactgagtagggaagcagcccgagccacagcgtcccctctagtccctcgcctccgagtcttcgcagatggccggttcagcctaacaagaggggctgggattcctgaagccccagtcccatgactcaggggttcctggcgggttccccctgccaggattccggtttctatcagcgaacgctcccctgccctgctccaggtccctcttgagtccgagttcccctacaggcctcggcctcagcctgcctagtcattctcgcttcgtctccctgactgtctgcaggcctggagagcatggccgtgttccgctcaggcctcctggtgctgacaacgccgctggccaccctggccgcacgcctgcctcccatcctgacctcggcgtcccgcctggtgaatcacaccctctatgtgcacctgcagccgggcatgaacctggggggacccgctcagcctcaggccagccctgtgcaggccacatttgaggttctggatttcatcacgcacctctacactggtgccgacctgcacaggcacctggacgtcagaattctactgaccaatatccaaaccaagagcacctttctccctgtgctgtcctcagtccagaatctggcccaccctccagaagtggttctgacggacttccagaccctggacgggagccagtacaaccctgttaaacaacagctagagcgttatgccaccagttgttacagctgttctccacagctggcttctgtgttgctgtatccggattatggaactggagagctgcctttggagcccccgaatgccctcttaccctctactataaggccggcttccccggttgccagatctccaaggcagcctgtgcgtggctaccaccgtggggccgtgggtggcacttttgaccgcttgcacaatgcccacaaagtgttgctcagtgttgcatgtgtactggcccaggagcagcttgtggtgggagtagctgacaaagatctgttgaagagtgagtgaagggaccctggactagggcaatggaaggcagccttcacattttcttgttcttccccaatgccaagatcaaggaagggtagggccacttctcaaccttccaaaatgccacaatgtctgacactctgttattgctaagggacgtttgtttgttaaatgaatacatgtggtatctttagtctggggttcagtcactttttaactgaagtcagaacttcccaaatgggaaccaaggactctgcacttcatggttgtccaagtgatcggctcggaggtttgaaacgctgaagataaatggataggagatgattgggggtgacccagtactgccatcaacagtgaggggactctaagaatatagtagttcttgggggttctccaccaactgatacctattggtcctgctgctctgtaggcatttcttaacttgttcttggagtacacgtcagctctcaaagtgactgatgcttgttttcccttctgtacccccaaacctgtccctgtctcaccctttcttgctgccactgcttgggaggatttcagtgctatgggacactcttccccaaatggggaaagatctcaccgttcctcgctccttcctcaggcaagttgctccccgagctgctccagccgtatgcagagcgcgtggagcatctgactgagttcctggtggacatcaagccctccctgacctttgaactcgtccccctgctggatccctatgggcctgctgggtccgaccccaccttggagttcctggtggtcagcgaggagacctaccgtggggggatggctgtcaaccgcttccgccttgagaatgtaactcccgaaggagcctggcagaggggctgggagggggaggcccgtgagaagtgctgtggtcccagagcagggaagggagggttcagggtggaagaacttgtcttgtcaagttcaggaacttgtctgtggaggcgaggggtaggtagagagactggggctccagatgccttctcctagctcttactcctttcctcctcctcctcctcctccctttacctcagggcaaagaagaacttgctttgtaccagatacaactgctgaaggaccagagtcacaatgaaaatgaggaggacaaggtcagctcctccagctttcgccagcgaatcctgggaaacctgcttcagcctccaaatgtaagccttcctcctcgctggttgggcgtggtaagatgctgaaggatagaaacaggccctccactggaagctgggcttttgaactccctggtcactgaggttccctcttacctgtctttaggagaggccggagctcccgtcaggtctctatgtgctcgggctcacaggcatcagtggctctgggaaaagctcggtagctcagcggctgaagaacttgggagcatacatcatcgacagcgaccatctgggccatcgggcctatgctcctgggggtcctgcctaccagcctgtggtggaggcctttggaacaggtactagccagagaaggctggaagtggcctgaggtgagaagatggtgtgacctctttgcttagccttctttctttgtcacccagatattctccataaagatggcaccatcaataggaaggtcctgggcagccgggtgtttgggaacaaggtaaacacacacatcttcaagagttcttgagatccatgggcgggaaccagtgcactccctagtctggcacaggaagtcagtctctgcttatctgctcccaccccaacccccacccccccaccccccacctcacagctgtatttctcaggaaagttaacctctgcccactccctctgtccccctccccagaagcagatgaagatcctcacagatattgtgtggccagttatcgcaaagctggcccgggaggagatggatgtggctgtggctaagggtgagtgggagggatgacgggagcagccaaagtgggacagttaagccgcaacaccctctgggatcttccttggccccaggcagaccattcaccacctaggacagcatttggtgcgctgacaggcagcaagcgcaaggtgctgctggcagtgactagtatctcctcacaggaaaaactctgtgtgtaatcgatgctgctatgctgcttgaagctggctggcagagtatggtacacgaggtgtggaccgtcgtcatccctgagactgaggtatctggaccccactgcaccccatctgagcccactgcagacggtctcctgctctgatccagtagactgggctaggacccttcctgacccgtgtgtcatccattgcccaggctgttcgacgcattgttgagagggatggactaagtgaggcagccgctcagagccggctgcagagccagatgagcggccagcagctggtagagcagagcaacgtggttctgagcaccttgtgggagtcacatgtcacacagagccaggttggtgcaaagggaaagactaggttaggttgtggggagggaaggttcaatggttagcttttctgaccttgccttttcttgtatcattctggcctggccaaaggtggaaaaggcgtggaatcttctacagaagcgcctccctaaggcttaccagaccaggaactgacagtggattttctatggcgccaaactggcccttgaaactaacaaatctaatggtgaggagaaatgggggccttgatgtttaccctctatctggcctcaggggctttaagctaagctgtgcaggacacagagaagcttggaagtccgcctagcatgctggaatttgtccagcaatgaggacacagctaatggggagcgatccgctctgaggttctgtccgctttttggtggtggatgtgatgtctacctagtgttgatgggtttggtgaagtctggacccagtaactgaattaaagatgtttccaagtgc

SEQ ID NO: 2 - DNA, 2153 bp, Mus musculus
gcctcgccccgccccaggagtctctcggcgatgctttgtttgattgctctgagttcccgtagctccacgctttgaggctggtgggtagtgcttccgggctgcagcctaggacgcctccacggcggtgcctggagagcatggccgtgttccgctcaggcctcctggtgctgacaacgccgctggccaccctggccgcacgcctgcctcccatcctgacctcggcgtcccgcctggtgaatcacaccctctatgtgcacctgcagccgggcatgaacctggggggacccgctcagcctcaggccagccctgtgcaggccacatttgaggttctggatttcatcacgcacctctacactggtgccgacctgcacaggcacctggacgtcagaattctactgaccaatatccaaaccaagagcacctttctccctgtgctgtcctcagtccagaatctggcccaccctccagaagtggttctgacggacttccagaccctggacgggagccagtacaaccctgttaaacaacagctagagcgttatgccaccagttgttacagctgttctccacagctggcttctgtgttgctgtatccggattatggaactggagagctgcctttggagcccccgaatgccctcttaccctctactataaggccggcttccccggttgccagatctccaaggcagcctgtgcgtggctaccaccgtggggccgtgggtggcacttttgaccgcttgcacaatgcccacaaagtgttgctcagtgttgcatgtgtactggcccaggagcagcttgtggtgggagtagctgacaaagatctgttgaagagcaagttgctccccgagctgctccagccgtatgcagagcgcgtggagcatctgactgagttcctggtggacatcaagccctccctgacctttgaactcgtccccctgctggatccctatgggcctgctgggtccgaccccaccttggagttcctggtggtcagcgaggagacctaccgtggggggatggctgtcaaccgcttccgccttgagaatggcaaagaagaacttgctttgtaccagatacaactgctgaaggaccagagtcacaatgaaaatgaggaggacaaggtcagctcctccagctttcgccagcgaatcctgggaaacctgcttcagcctccaaatgagaggccggagctcccgtcaggtctctatgtgctcgggctcacaggcatcagtggctctgggaaaagctcggtagctcagcggctgaagaacttgggagcatacatcatcgacagcgaccatctgggccatcgggcctatgctcctgggggtcctgcctaccagcctgtggtggaggcctttggaacagatattctccataaagatggcaccatcaataggaaggtcctgggcagccgggtgtttgggaacaagaagcagatgaagatcctcacagatattgtgtggccagttatcgcaaagctggcccgggaggagatggatgtggctgtggctaagggaaaaactctgtgtgtaatcgatgctgctatgctgcttgaagctggctggcagagtatggtacacgaggtgtggaccgtcgtcatccctgagactgaggctgttcgacgcattgttgagagggatggactaagtgaggcagccgctcagagccggctgcagagccagatgagcggccagcagctggtagagcagagcaacgtggttctgagcaccttgtgggagtcacatgtcacacagagccaggtggaaaaggcgtggaatcttctacagaagcgcctccctaaggcttaccagaccaggaactgacagtggattttctatggcgccaaactggcccttgaaactaacaaatctaatggtgaggagaaatgggggccttgatgtttaccctctatctggcctcaggggctttaagctaagctgtgcaggacacagagaagcttggaagtccgcctagcatgctggaatttgtccagcaatgaggacacagctaatggggagcgatccgctctgaggttctgtccgctttttggtggtggatgtgatgtctacctagtgttgatgggtttggtgaagtctggacccagtaactgaattaaagatgtttccaagtgcaaaaaaaaaaaaaaa

SEQ ID NO: 3 - DNA, 2479 bp, Homo sapiens
gttagtgcttccgggttgcagccagggaagcctccgcggtggtgcaagtggaacccaagccttgaggtttcagtgagtagggggccgacgtgagctttagcgtccccctttagcctccctcttcgattccttgaagaccctggtgcagcttagcaagagggcccaggatttttggatccccagccctgtgacaagggttcctgtccagtttccccctcccaggatttcgactcagttcagcgaagtcaccgccccgtctgagaaatgaggacaccaaggcttagagcacagccccgaggcgccgtctaccaggccccgtcccctcccccggctcctgtcggtcagcactgaaaccccgtccctgctccaggcctccttctctggggtccaaggtcccatacaggcctctgcctcggccgcaggcccttcagtcaccgtcgcctcgtctccctgactgtccgcaggcctgggcagcatggccgtattccggtcgggtctcctggtgctgacgacgccgctggcctccctagcccctcgcctggcctccatcctgacctcggcggcccggctggtgaatcacacactctatgttcacctgcagccgggcatgagcctggagggcccggctcagccccagtccagccccgtgcaggccacgtttgaggttcttgatttcatcacgcacctctatgctggcgccgacgtccacaggcacttggacgtcagaatcctactgaccaatatccgaaccaagagcacctttctccctcccctgcccacctcagtccagaatctcgcccacccgccagaagtcgtgttgacagatttccagaccctggatggaagccagtacaacccggtcaaacagcagctagtgcgttacgccaccagctgttacagctgttgtccgcgactggcctcggtgctgctatactccgattatgggataggagaagtgcccgtggagcccctggatgtccccttaccctccacgatcaggccagcttcccccgtggccgggtctccaaagcagccggtgcgtggctactaccgtggcgctgtcggtggcacgtttgaccgcctgcacaacgcccacaaggtgttgctcagtgtcgcgtgcatcctggcccaggagcagcttgtggtgggagtagcagacaaagatctgttgaagagcaagttgctccctgagctgctccaaccttatacagaacgtgtggaacatctgagtgaattcctggtggacatcaagccctccttgacttttgatgtcatccccctgctggacccctatgggcccgctggctctgacccctccctggagttcctggtggtcagcgaggagacctatcgtggggggatggccatcaaccgcttccgccttgagaatgacctggaggaacttgctttgtaccagatccagctgctgaaggacctcagacatacagagaatgaagaggacaaagtcagctcctccagcttccgccagcgaatgttggggaacctgcttcggcctccatatgaaaggccagagctccccacatgtctctatgtaattgggctgactggcatcagtggctctgggaagagctcaatagctcagcgactgaagggcctgggggcgtttgtcattgacagtgaccacctgggtcatcgggcctatgccccaggtggccctgcctaccagcctgtggtggaggcctttggaacagatattctccataaagatggcatcatcaacaggaaggtcctaggcagccgggtgtttgggaataagaagcagctgaagatactcacggacattatgtggccaattatcgcaaagctggcccgagaggagatggatcgggctgtggctgagggaaagcgtgtgtgtgtgattgatgccgctgtgttgcttgaagccggctggcagaacctggtccatgaggtgtggactgctgtcatcccagagactgaggctgtaagacgcattgtggagagggatggcctcagtgaagccgcggctcaaagccggctgcagagccagatgagcgggcagcagcttgtggaacagagccacgtggtgctcagcaccttgtgggagccgcatatcacccaacgccaggtggagaaagcctgggccctcttgcagaagcgcattcccaagactcatcaggccctcgactgaaaagttctcagtggggccagactggctcctggagctgacaagcgaccccgtggtgaggagaaatgggggccttgatgctcaccctggttcaggcccagaggtccaagctatactgtgcaggacatggccaggcctggtggacacaggaagcctacccaacacgctggtatttggccaacactgaggatgtggttcatgggggagcagtcccctccccactcttgcccatgggtgactcttacccacagctgactagggccagcgcaaatactggaacctgtaacagaattaaaggtgaatgttctgaga

SEQ ID NO: 4 - DNA, 11208 bp, Homo sapiens
ccaattccacatttccagtcctcctaccggctcctcccccagtcttctctactttactgtatgacaactccatttttcccaatactggttttaaaaaccctggagtagtccttgactccagtctctctcacctttcaaccaattcagcagcaaatactgacagctctacctttgctcatcaaagcatattccaaaatcaccctattcgagactgcacactgtcatctttcccttcaattacagcagtagcctcctaactagttttcttgattccactcttgcccatcagcagtaaaattagcacagtgcatttaaaatgatcttttaaaaatgacagggccgggcgtgatggctcatgcctataatcacagcactttgtgaggctgaggcggacgttattgcttgaggtcaggagtttgagaccaacctggccaatatggtgaaaccctgtgtctactaaaaatataaaaattagccaggcatggtggtgggcgcctgtaatcgcagctgctccagaggctgacaaaggagaatctcttgaaccagggaggtggaggctgcagtgagccaagattgggacactgcactccagcctgggcaacagagactttgtctcaaaaaaataaaaattggccgggcgtggtggctcatgcctgtaatcccagcactttgggaggccgaggcgggtggatcacctgaggtcaggagttcaagaccagcctggccaacagggtgaaaccttgtctctactaaaaacccaaaaaattagccaggcgtggtggcacgtgcctgtaatcccagctactcaggcggctgaggcaggggaattgcttgaaccccggaggcgtacgctgcagtgagtggagattgcgccattgtactctagcctgggcaacaagagtgaaactccatctcaaaataataataataaaattaaaaaaaatttaaaataaaaaagggagcctgggcaacaaagtgagaccccgtctctacgaaaaaacaaaaaaaatagttgggtgtggttgcacacatctgtagtcccagcttcttgggaggctgacatgggaagactgtttgagcccaggaggttgaggccacagtaagctgtgggcacaccactgcattccagcctgggcaagagagggagaccctgtctcataaaagaaagaaagaggccaggtgtggtggctacgcctgtaatcccaacactttgggagcccaaggccggtgaatcacttgagatcaggagttcgagaccagcctggccaacatggtaaaaccccgtctctactaaaaatacaaaaattagctgggcgtggtagtttgtgcctgtaatcccagctactcgggaggctgaggcaggagaatcgcttgaacccgggagacgaaggttgcagtgagctgagatttctccactgcactccagcctgggtaaagagtgagactcggccgggtgcggtggctcatgtctttaatcccagcactttgggaggccgaggcgggtggatcacctgaggtcaggagttcaagaccagcctggccaacctggtgaaagcctgtctctactaaaaatagaaaaattagctgggcgtggtggtgggtgcctgtaatcccagctactcaggaggctgaagcaggagaatcacttgaacctgggaggcagaggttgcagtgagccgagatcatgccgttgcactccaacctgggtgacagtgagactctgtcttaaaacaaaacaaaagaagcaaagtttattttgctcagagcttcacagttttaggggctggaaagtgcaagaacttcatgcagtatctggcaaagggctgtcccaggatggaagggcagaaggtggaagcaaatgagagacacgaaattgggccaaatgtcgtccatccatcaggagccactcccatgataacagtatttatctattcaagagggcagagccctcatgatctaattatcttttttttttttctcctgcctcggcctcttgagaagctgggattttaggcgcatgccaccacaccccgctaatttttgtacttttagtagagacggggtttcaccacgttggttgggctggtcgcaaactcctgatctcaggtgatccacctgagatccaccactttggcttcccaaagtgctgagattacaggagtgagccacctcgcccagctgacctaattacctcttaaaggccccaccctgcccagtgcagtggctcaggcctgtaatcccaacactttgggaggctgaagtgggaggactgcttgagttcaggagttggaaaccagcccaggcaacacagcaagatcctgtctgtatgaaaaaaaaaattttttttttaattagccaggtgtggtggcacacatctgtagttccagctgctcaggaggccaaggcagaggactgcttgagcccaggcagttgaggctgctgtgagccatgatcaacccactgcactccagcctgggggacaagagtgagaccctgtctcaaagagaaaaaaagtcccatctcttaagacagtagcaattgaattttttatttcaacatgagttgtggcagggacattcgaaccatagcaccaaggttctccccaaagctctccaagaccttctttgatctggttcctgccccctttctgatctcatctcccaccaatctccatctacatcacccagttcagccctcctgcctcagggcctttgtacttggccccctctgggtcacattccctgatctctgattctatgtcttccttcagatttttgctgttttcctgaaccaccttatctaaaatagcctctcagcttctccactatagctcccttactctgctttacttttgttcttatcctttatcaccagtagatatattatggatttgggttttttaatttaatttttattttttttggagacggagtctctctgtcacccaggctggagtgcagtggcgtgatctcggctcattgcaatctctgcctcccaggttcaagcgattctcctgcctcagcctcctgagtagctggaatcacaagtaccgccaccacacccagctaattttttgtatttttagtagagacggggtttcactatgttggccaggctagtcttgaactcctgacctcaggtgatctgcctgcctcagcttcccaaagtgctgggattacaggcgtgagccaccgcgcctggccatgtattatagatttatttatggtggcatgattcctgtctgcaaccccaagacacacagtactctataccccaggatggcagggacattgttttgttcactggtccatccctgtgggaggcagtaaaatacacatttactcaataaaaatgactctgtatgtggtgtgtcagactgtgtgtccagatgtcttgggacattctctggcagcctttgctgtttgtgtcttcaaatcagaccctatctttaaatctaacatgtctcctgtgagaagcctggtcccttaatacctttgtggctctacaactcttccctccttgactgtatctcttcctccaggtctttgctgttttcctgaatcaccttatctaaaatagcatctccttttctccatcataagcccaggttttgtggggctgggggcttatttgcttttggagaccgtcttcaagaatacaaaagataaacacaaaattacatataaaagtgagttttgtttttttttttaagtttcggataccacggcacattactggaatcttggagactggcttctctcccttttgatctccatgcacaacttacctgagatgtattctaatagcaacctggcttcccctccacactcagcctttcaagcgcccattagcttcgcagtaaatctcccacactaggtatccagcctttgcctttttcttttttctttttttttttccagagggagtttcgcttttttttttttttttttttttttgagacggagtctcactctgtcacccaggctggagtgtagtggtgcgatctcggctcactgcaagctcttcctcccgggttcacgccattctcctgcctcagcctcccgagtagctgggactacaggcgtctgccaccatgcccggctaatttttttttgtatttttagtagagacggggtttcaccgtgttagccaggatggtctcgatctcctgacctcgtgatccgcccgcctcggcctcccaaagtgctgggattacaggcgtcagccaccgcgcccggccgggagtttcgctcttgttgcccaggttggagtgcaatggcacaatcttggctcaccgcgacctccgcccacgggttcaggcgattctcctgcctcagcctcctgagtagctgggattacagccatgcgccaccacgctcgtctaattttgtatttttagtagagacaaggtttctccatattggtcaggttggtctcgaactcccgacctcagatgctctgcccacctcggcatcccaaagtgctgggattacaggcgtgagccaccgcgcccaatcagcctttgcctttttctcctgtccacagagaccccgcaaacaagcgcatcagaggatctgggtggtgcgagagcctcctacagcagccaggtacacacaaggcctcagtttttccatctgtgaaatgggtacgtggacagacctgtccagtagcctcccatgtgagaatcctcggactgagagggtactaggagggctaatacagcgcagccctgtgattccccacctctgtccctggaatgaaggaaatctgggtgttagttctggctttgccgatgcctcttgggtggcctggctgaaactaatctctcctcatcaggccagaagggaatctgtgaaatgggctttgaaacctgccttgcctttctccgcccggtcccagcgggggtccgatcggcatcaaggcttcacccactcccagagccttcgggatcagccccaccccatcagcaggacacagcccatgccactggaaatctcgcgagaatcccgcgctgaagcgaagtcgagacgtttgaccttcctccgtttccgtagttccgagttgcggtttctccgttagtgcttccgggttgcagccagggaagcctccgcggtggtgcaagtggaacccaagccttgaggtttcagtgagtagggggccgacgtgagctttagcgtccccctttagcctccctcttcgattccttgaagaccctggtgcagcttagcaagagggcccaggatttttggatccccagccctgtgacaagggttcctgtccagtttccccctcccaggatttcgactcagttcagcgaagtcaccgccccgtctgagaaatgaggacaccaaggcttagagcacagccccgaggcgccgtctaccaggccccgtcccctcccccggctcctgtcggtcagcactgaaaccccgtccctgctccaggcctccttctctggggtccaaggtcccatacaggcctctgcctcggccgcaggcccttcagtcaccgtcgcctcgtctccctgactgtccgcaggcctgggcagcatggccgtattccggtcgggtctcctggtgctgacgacgccgctggcctccctagcccctcgcctggcctccatcctgacctcggcggcccggctggtgaatcacacactctatgttcacctgcagccgggcatgagcctggagggcccggctcagccccagtccagccccgtgcaggccacgtttgaggttcttgatttcatcacgcacctctatgctggcgccgacgtccacaggcacttggacgtcagaatcctactgaccaatatccgaaccaagagcacctttctccctcccctgcccacctcagtccagaatctcgcccacccgccagaagtcgtgttgacagatttccagaccctggatggaagccagtacaacccggtcaaacagcagctagtgcgttacgccaccagctgttacagctgttgtccgcgactggcctcggtgctgctatactccgattatgggataggagaagtgcccgtggagcccctggatgtccccttaccctccacgatcaggccagcttcccccgtggccgggtctccaaagcagccggtgcgtggctactaccgtggcgctgtcggtggcacgtttgaccgcctgcacaacgcccacaaggtgttgctcagtgtcgcgtgcatcctggcccaggagcagcttgtggtgggagtagcagacaaagatctgttgaagagtgagtaagagggaccctggactagggtggaggatccccaaatctccccacccccgacccttatgccgagatcaaggaagggtagggccattcattacttcccacccttcccaaatgtcacagtggttgacactcaattggtgctaaggccattttgtcaaatgaacagctttctcagcatgtggtccagtcaccactcaatcagaataccccaaatgtgaagcaaagggatctgcattttagcaagttttccaggtgatacaccctaatgagacactgggaatgaatgggtgagtgagctgcaggtagcagtgccacagacaggaggaggaaactcaagcatggcatgggtcttggaattttccatctgcctctgatgccctctggcactgctcgttctctggagtggtttcctggtggcttattctctggacacatgccagcccttggagtgactattgtgcttgcctgtttcttcaccttcatgctcccctcaccatcaccataggccttaccagttgaaccctttctgccaccccctctggggatactgtacttagggacactttttcccaaactggcccatactctcctccccaataaaaagatctcactgttcttctgggctccttccccaggcaagttgctccctgagctgctccaaccttatacagaacgtgtggaacatctgagtgaattcctggtggacatcaagccctccttgacttttgatgtcatccccctgctggacccctatgggcccgctggctctgacccctccctggagttcctggtggtcagcgaggagacctatcgtggggggatggccatcaaccgcttccgccttgagaatgtaacccctgagggagactggcagagggagtggatgggggacggggaaggccattttgagggggctgttggaagtaccatggccccagaggagagaagtgggggctcagggtggtgggaagaagcaaggaggaactggttctcagtttgcccgctgagtcggaggaggaggagcctgggtaggaaggggaggatgggggggcaggttggatgtcagggtcttcctctcactccctccttccctctttttaccctttcctctttaccccaggacctggaggaacttgctttgtaccagatccagctgctgaaggacctcagacatacagagaatgaagaggacaaagtcagctcctccagcttccgccagcgaatgttggggaacctgcttcggcctccatatgtaagctcctctccctccttccctcctgcttggtgtcctggcaatgctggagagtagaagctgaggggctcagccccaggcatgaggctgagggccccagtaactgtgggttccctttcacctatccccaggaaaggccagagctccccacatgtctctatgtaattgggctgactggcatcagtggctctgggaagagctcaatagctcagcgactgaagggcctgggggcgtttgtcattgacagtgaccacctgggtcatcgggcctatgccccaggtggccctgcctaccagcctgtggtggaggcctttggaacaggtaataactggggaaggctgaaagtggcctggagtgaggagctagccaggcctctgtgctcagttgtctgtctgtgttgtccagatattctccataaagatggcatcatcaacaggaaggtcctaggcagccgggtgtttgggaataaggtaaacaataacttcctaagggctcctaagccgctactagacccaggggtcagggtccagtggacctctctggtctggcccagaatgccatttccattgatctgttcccaacaccaccttgctcgggctggctgcctcttctagagaaggtaacctctgccctctgttcccctccccagaagcagctgaagatactcacggacattatgtggccaattatcgcaaagctggcccgagaggagatggatcgggctgtggctgagggtgagtgggagggaggatggcaacagctaaggggacagttaagctgtttcttcccctgggagcttccctgcccaacgtgggactgtctgttcaccctgggactgtgttctgcctgggagaacgtcggcactgctggcggtgactggggtctccccacaggaaagcgtgtgtgtgtgattgatgccgctgtgttgcttgaagccggctggcagaacctggtccatgaggtgtggactgctgtcatcccagagactgaggtatctcgccccaccccccacaccatccctactgcagatcctatcctgtgagctggaattcttcctgacaaatgtctcgtctgtgctcaggctgtaagacgcattgtggagagggatggcctcagtgaagccgcggctcaaagccggctgcagagccagatgagcgggcagcagcttgtggaacagagccacgtggtgctcagcaccttgtgggagccgcatatcacccaacgccaggttggtgcccagggcaaggccgggttgtggggaaggagtctccagtgggtactgcctgaccctgccctctcttcctcccaacatcctggcctgtctgaaggtggagaaagcctgggccctcttgcagaagcgcattcccaagactcatcaggccctcgactgaaaagttctcagtggggccagactggctcctggagctgacaagcgaccccgtggtgaggagaaatgggggccttgatgctcaccctggttcaggcccagaggtccaagctatactgtgcaggacatggccaggcctggtggacacaggaagcctacccaacacgctggtatttggccaacactgaggatgtggttcatgggggagcagtcccctccccactcttgcccatgggtgactcttacccacagctgactagggccagcgcaaatactggaacctgtaacagaattaaaggtgaatgttctgagatgttgcttgtatggtgtctgcttcctgttccaatgcccctaagtatctttgggatccttaagccacaagtgcagggcccaggcctgagctggaagcctgggcaatgcagtggttaagagaatggcctttgaggctagccagaggcgagctttggccctcccactgaccagctgtgttcttctgagtaaggtgtagactttctaggtactgccgtgttttattgtttttgttttgtttttaatatgagaggccgcataacctagctagcaggttctttgcaagggttcattccacatgcatcctgccagacactgggctgggtgttggaagatcagccttccgcgcgccaggcggggcctctgccctcacagagcttgcagcctggtggggtcggacgatgtggctgggggcttctctgcccggttcctgacacaccggccagcagtgcaggtccggtgagcgcggggaggccgccctcagggtaacgtggggagccggccggccggacagtgcgcgtgcacacactatctgcggcggtgtgctgacaagccaattgaggggactccatcttacaaaacgcgcttcatgtcgccgccattccactgcctaacccggggagggcaggcacggcggggagcagtctcgaaggaggctggcgccaggccaagcgccgggccttgacggctggaaccgtggagtaaagtctttgggctgcgcatgcgcgatgcttcgccctgccccacctctcggtcgcggattggcgggcgcgggtcacgtgggcacgccacccgcttcctcgccgcagggggcccgcccgctggcccgtttccggtccggtgggtacaagatgacggagccgggcgcctctcccgaggacccttgggtcaaggcaagccccgtgggcgcgcacgccggcgaggggagggcgggtcgggctcgtgcacgtaggggggccggaagacgaggggcttccctcctgtccccaaagtcccccacgctctccgtgccccggggctgcagagaagacagctctcaccccgcgtgtgccaaggtggggagacaaacgaggcgtgtgcgcgcgagtcgggggaacggggcactggggtggagtaggggcggaagggatcatacacaggggaggcactcgcacgccctagcctgtgccagtctcgggggactcattaagctgcccgcgcaccccgggctgtgtgtgtgcaagcgcgcagggtgacagaggcgccttcccggaatggggggcgcgctgtgcgtgcctgcagtggaaggggcgcctccccctaggggcggaggtctgacgggcccttcccgtgctctgtgccgcaggtggagtatgcctacagcgacaacagcctggaccccggtgagtagctgccccatcttaagctctagagggacactcccgcccaggctctctagattcttgtggcgttgccaaccacgcctgagcacagtccactctccctgagcagagttcctggcttgagacagcaggagctgaaggacaagggcagaaaacaactgagggtgacacctagttccctctgctctggaacagggaagtggtggaactgatcactcaccactcagtggagcagctgatcactcacaccctcatcacgtccacatgcccgtttgtgagccaggggggtatcatgcaggcctatggagcagtgtagacactagggagcagagcactgcagcgtggtctggcaggaagtggggggcccagtatggttggctttgggttgaagggaagaggtcagcatttaagaaagccattgtgtggccgggcgcggtggctcacccctgtaatcccagcactttgggaggccgaggcaggcggatcacgaggtcaggagaccgagaccatcctggctaacacggtgaaaccccgtctccactaaaaaatacaaaaaaattagccgggcgtggtggcgggcgcctgtagtcccagctactcgggaggctgaggcaggagaatggcgt

SEQ ID NO: 5 - DNA, 20 bp, Artificial sequence
  Primer
tgcttcagcctccaaatgag

SEQ ID NO: 6 - DNA, 22 bp, Artificial sequence
  Primer
tgtatgctcccaagttcttcag

SEQ ID NO: 7 - DNA, 24 bp, Artificial sequence
  Probe
tcccgtcaggtctctatgtgctcg

SEQ ID NO: 8 - DNA, 15 bp, Artificial sequence
  Primer
tcgccgcttgctgca

SEQ ID NO: 9 - DNA, 17 bp, Artificial sequence
  Primer
atcggccgtgatgtcga

SEQ ID NO: 10 - DNA, 23 bp, Artificial sequence
  Probe
ccatggtcaaccccaccgtgttc

SEQ ID NO: 11-14 - skipped (4 entries)

SEQ ID NO: 15 - DNA, 16 bp, Artificial sequence
  Synthetic oligonucleotide
gaccaataggtatcag

SEQ ID NO: 16 - DNA, 16 bp, Artificial sequence
  Synthetic oligonucleotide
gagtaagagctaggag

SEQ ID NO: 17 - DNA, 16 bp, Artificial sequence
  Synthetic oligonucleotide
gcacatagagggtgtg

SEQ ID NO: 18 - DNA, 16 bp, Artificial sequence
  Synthetic oligonucleotide
gcgaagactcggaggc

SEQ ID NO: 19 - DNA, 16 bp, Artificial sequence
  Synthetic oligonucleotide
gccaaggaagatccca

SEQ ID NO: 20 - DNA, 16 bp, Artificial sequence
  Synthetic oligonucleotide
aaaaagcggacagaac

SEQ ID NO: 21 - DNA, 16 bp, Artificial sequence
  Synthetic oligonucleotide
gcattttggaaggttg

SEQ ID NO: 22 - DNA, 16 bp, Artificial sequence
  Synthetic oligonucleotide
tcctaaagacaggtaa

SEQ ID NO: 23 - DNA, 16 bp, Artificial sequence
  Synthetic oligonucleotide
gcacttggaaacatct

SEQ ID NO: 24 - DNA, 16 bp, Artificial sequence
  Synthetic oligonucleotide
cagcatagcagcatcg

SEQ ID NO: 25 - DNA, 16 bp, Artificial sequence
  Synthetic oligonucleotide
ggctagtacctgttcc

SEQ ID NO: 26 - DNA, 16 bp, Artificial sequence
  Synthetic oligonucleotide
taatccggatacagca

SEQ ID NO: 27 - DNA, 16 bp, Artificial sequence
  Synthetic oligonucleotide
aaccatgaagtgcaga

SEQ ID NO: 28 - DNA, 16 bp, Artificial sequence
  Synthetic oligonucleotide
tcaaacaaagcatcgc

SEQ ID NO: 29 - DNA, 16 bp, Artificial sequence
  Synthetic oligonucleotide
gagctacgggaactca

SEQ ID NO: 30 - DNA, 16 bp, Artificial sequence
  Synthetic oligonucleotide
gcacatagagacctga

SEQ ID NO: 31 - DNA, 16 bp, Artificial sequence
  Synthetic oligonucleotide
agaatatctgggtgac

SEQ ID NO: 32 - DNA, 16 bp, Artificial sequence
  Synthetic oligonucleotide
gaactactatattctt

SEQ ID NO: 33 - DNA, 16 bp, Artificial sequence
  Synthetic oligonucleotide
caataacagagtgtca

SEQ ID NO: 34 - DNA, 16 bp, Artificial sequence
  Synthetic oligonucleotide
caaaagtgccacccac

SEQ ID NO: 35 - DNA, 16 bp, Artificial sequence
  Synthetic oligonucleotide
caacagatctttgtca

SEQ ID NO: 36 - DNA, 16 bp, Artificial sequence
  Synthetic oligonucleotide
gtttaccttgttccca

SEQ ID NO: 37 - DNA, 16 bp, Artificial sequence
  Synthetic oligonucleotide
taggtatcagttggtg

SEQ ID NO: 38 - DNA, 16 bp, Artificial sequence
  Synthetic oligonucleotide
acaaattccagcatgc

SEQ ID NO: 39 - DNA, 16 bp, Artificial sequence
  Synthetic oligonucleotide
gttaaaaagtgactga

SEQ ID NO: 40 - DNA, 16 bp, Artificial sequence
  Synthetic oligonucleotide
cccatagggatccagc

SEQ ID NO: 41 - DNA, 16 bp, Artificial sequence
  Synthetic oligonucleotide
aaccattgaaccttcc

SEQ ID NO: 42 - DNA, 16 bp, Artificial sequence
  Synthetic oligonucleotide
cttatagtagagggta

SEQ ID NO: 43 - DNA, 16 bp, Artificial sequence
  Synthetic oligonucleotide
acgaagcgagaatgac

SEQ ID NO: 44 - DNA, 16 bp, Artificial sequence
  Synthetic oligonucleotide
tcttaccacgcccaac

SEQ ID NO: 45 - DNA, 16 bp, Artificial sequence
  Synthetic oligonucleotide
gggtaaacatcaaggc

SEQ ID NO: 46 - DNA, 16 bp, Artificial sequence
  Synthetic oligonucleotide
gatactagtcactgcc

SEQ ID NO: 47 - DNA, 16 bp, Artificial sequence
  Synthetic oligonucleotide
cccaagcagtggcagc

SEQ ID NO: 48 - DNA, 16 bp, Artificial sequence
  Synthetic oligonucleotide
ccatagaaaatccact

SEQ ID NO: 49 - DNA, 16 bp, Artificial sequence
  Synthetic oligonucleotide
cgagaatgactaggca

SEQ ID NO: 50 - DNA, 16 bp, Artificial sequence
  Synthetic oligonucleotide
caccaaaaagcggaca

SEQ ID NO: 51 - DNA, 16 bp, Artificial sequence
  Synthetic oligonucleotide
cattagatttgttagt

SEQ ID NO: 52 - DNA, 16 bp, Artificial sequence
  Synthetic oligonucleotide
cgattacacacagagt

SEQ ID NO: 53 - DNA, 16 bp, Artificial sequence
  Synthetic oligonucleotide
cacaatatctgtgagg

SEQ ID NO: 54 - DNA, 16 bp, Artificial sequence
  Synthetic oligonucleotide
ccccattagctgtgtc

SEQ ID NO: 55 - DNA, 16 bp, Artificial sequence
  Synthetic oligonucleotide
gagcaacttgcctgag

SEQ ID NO: 56 - DNA, 16 bp, Artificial sequence
  Synthetic oligonucleotide
taagccttagggaggc

SEQ ID NO: 57 - DNA, 16 bp, Artificial sequence
  Synthetic oligonucleotide
gatcagagcaggagac

SEQ ID NO: 58 - DNA, 16 bp, Artificial sequence
  Synthetic oligonucleotide
acaagacaagttcttc

SEQ ID NO: 59 - DNA, 16 bp, Artificial sequence
  Synthetic oligonucleotide
tgatacaagaaaaggc

SEQ ID NO: 60 - DNA, 16 bp, Artificial sequence
  Synthetic oligonucleotide
ggagaaggcatctgga

SEQ ID NO: 61 - DNA, 16 bp, Artificial sequence
  Synthetic oligonucleotide
ggcgaaagctggagga

SEQ ID NO: 62 - DNA, 16 bp, Artificial sequence
  Synthetic oligonucleotide
atcgccgagagactcc

SEQ ID NO: 63 - DNA, 16 bp, Artificial sequence
  Synthetic oligonucleotide
ccagactagggagtgc

SEQ ID NO: 64 - DNA, 16 bp, Artificial sequence
  Synthetic oligonucleotide
aagctaaccattgaac

SEQ ID NO: 65 - DNA, 16 bp, Artificial sequence
  Synthetic oligonucleotide
aagataccacatgtat

SEQ ID NO: 66 - DNA, 16 bp, Artificial sequence
  Synthetic oligonucleotide
cgctgatagaaaccgg

SEQ ID NO: 67 - DNA, 16 bp, Artificial sequence
  Synthetic oligonucleotide
agcggaacacggccat

SEQ ID NO: 68 - DNA, 16 bp, Artificial sequence
  Synthetic oligonucleotide
ccttagcaataacaga

SEQ ID NO: 69 - DNA, 16 bp, Artificial sequence
  Synthetic oligonucleotide
tttaacagggttgtac

SEQ ID NO: 70 - DNA, 16 bp, Artificial sequence
  Synthetic oligonucleotide
agactaaagataccac

SEQ ID NO: 71 - DNA, 16 bp, Artificial sequence
  Synthetic oligonucleotide
gtagaagattccacgc

SEQ ID NO: 72 - DNA, 16 bp, Artificial sequence
  Synthetic oligonucleotide
cactttgagagctgac

SEQ ID NO: 73 - DNA, 16 bp, Artificial sequence
  Synthetic oligonucleotide
taacaactggtggcat

SEQ ID NO: 74 - DNA, 16 bp, Artificial sequence
  Synthetic oligonucleotide
cactaggtagacatca

SEQ ID NO: 75 - DNA, 16 bp, Artificial sequence
  Synthetic oligonucleotide
taattcagttactggg

SEQ ID NO: 76 - DNA, 16 bp, Artificial sequence
  Synthetic oligonucleotide
aaacctccgagccgat

SEQ ID NO: 77 - DNA, 16 bp, Artificial sequence
  Synthetic oligonucleotide
agcttaaagcccctga

SEQ ID NO: 78 - DNA, 16 bp, Artificial sequence
  Synthetic oligonucleotide
aaacatcaaggccccc

SEQ ID NO: 79 - DNA, 16 bp, Artificial sequence
  Synthetic oligonucleotide
caagttaagaaatgcc

SEQ ID NO: 80 - DNA, 16 bp, Artificial sequence
  Synthetic oligonucleotide
aacccatcaacactag

SEQ ID NO: 81 - DNA, 16 bp, Artificial sequence
  Synthetic oligonucleotide
agaacctcaaatgtgg

SEQ ID NO: 82 - DNA, 16 bp, Artificial sequence
  Synthetic oligonucleotide
ctaacctagtctttcc

SEQ ID NO: 83 - DNA, 16 bp, Artificial sequence
  Synthetic oligonucleotide
attcttagagtcccct

SEQ ID NO: 84 - DNA, 16 bp, Artificial sequence
  Synthetic oligonucleotide
agctaccgagcttttc

SEQ ID NO: 85 - DNA, 16 bp, Artificial sequence
  Synthetic oligonucleotide
agatagagggtaaaca

SEQ ID NO: 86 - DNA, 16 bp, Artificial sequence
  Synthetic oligonucleotide
ttaactttcctgagaa

SEQ ID NO: 87 - DNA, 16 bp, Artificial sequence
  Synthetic oligonucleotide
taaagcccctgaggcc

SEQ ID NO: 88 - DNA, 16 bp, Artificial sequence
  Synthetic oligonucleotide
taagcaaagaggtcac

SEQ ID NO: 89 - DNA, 16 bp, Artificial sequence
  Synthetic oligonucleotide
ttccataatccggata

SEQ ID NO: 90 - DNA, 16 bp, Artificial sequence
  Synthetic oligonucleotide
cagtagaattctgacg

SEQ ID NO: 91 - DNA, 16 bp, Artificial sequence
  Synthetic oligonucleotide
gaagcgagaatgacta

SEQ ID NO: 92 - DNA, 16 bp, Artificial sequence
  Synthetic oligonucleotide
gcttaaagcccctgag

SEQ ID NO: 93 - DNA, 16 bp, Artificial sequence
  Synthetic oligonucleotide
aacaactggtggcata

SEQ ID NO: 94 - DNA, 16 bp, Artificial sequence
  Synthetic oligonucleotide
gccagatagagggtaa

SEQ ID NO: 95 - DNA, 16 bp, Artificial sequence
  Synthetic oligonucleotide
ccacaacctaacctag

SEQ ID NO: 96 - DNA, 16 bp, Artificial sequence
  Synthetic oligonucleotide
tagaagattccacgcc

SEQ ID NO: 97 - DNA, 16 bp, Artificial sequence
  Synthetic oligonucleotide
gaacccccaagaacta

SEQ ID NO: 98 - DNA, 16 bp, Artificial sequence
  Synthetic oligonucleotide
caccattagatttgtt

SEQ ID NO: 99 - DNA, 16 bp, Artificial sequence
  Synthetic oligonucleotide
tcctattgatggtgcc

SEQ ID NO: 100 - DNA, 16 bp, Artificial sequence
  Synthetic oligonucleotide
aaaagtgccacccacg

SEQ ID NO: 101 - DNA, 16 bp, Artificial sequence
  Synthetic oligonucleotide
gatagagggtaaacat

SEQ ID NO: 102 - DNA, 16 bp, Artificial sequence
  Synthetic oligonucleotide
agatacctcagtctca

SEQ ID NO: 103 - DNA, 16 bp, Artificial sequence
  Synthetic oligonucleotide
gcgccatagaaaatcc

SEQ ID NO: 104 - DNA, 16 bp, Artificial sequence
  Synthetic oligonucleotide
agataccacatgtatt

SEQ ID NO: 105 - DNA, 16 bp, Artificial sequence
  Synthetic oligonucleotide
caggtaagagggaacc

SEQ ID NO: 106 - DNA, 16 bp, Artificial sequence
  Synthetic oligonucleotide
cattagctgtgtcctc

SEQ ID NO: 107 - DNA, 16 bp, Artificial sequence
  Synthetic oligonucleotide
cattttggaaggttga

SEQ ID NO: 108 - DNA, 16 bp, Artificial sequence
  Synthetic oligonucleotide
gactcggaggcgaggg

SEQ ID NO: 109 - DNA, 16 bp, Artificial sequence
  Synthetic oligonucleotide
actatattcttagagt

SEQ ID NO: 110 - DNA, 16 bp, Artificial sequence
  Synthetic oligonucleotide
gctgatagaaaccgga

SEQ ID NO: 111 - DNA, 16 bp, Artificial sequence
  Synthetic oligonucleotide
gattacacacagagtt

SEQ ID NO: 112 - DNA, 16 bp, Artificial sequence
  Synthetic oligonucleotide
gcatagcagcatcgat

SEQ ID NO: 113 - DNA, 16 bp, Artificial sequence
  Synthetic oligonucleotide
gaacttgacaagacaa

SEQ ID NO: 114 - DNA, 16 bp, Artificial sequence
  Synthetic oligonucleotide
aaaaagtgactgaacc

SEQ ID NO: 115 - DNA, 16 bp, Artificial sequence
  Synthetic oligonucleotide
aatgactaggcaggct

SEQ ID NO: 116 - DNA, 16 bp, Artificial sequence
  Synthetic oligonucleotide
aagccttagggaggcg

SEQ ID NO: 117 - DNA, 16 bp, Artificial sequence
  Synthetic oligonucleotide
agctacgggaactcag

SEQ ID NO: 118 - DNA, 16 bp, Artificial sequence
  Synthetic oligonucleotide
agctaaccattgaacc

SEQ ID NO: 119 - DNA, 16 bp, Artificial sequence
  Synthetic oligonucleotide
acaatatctgtgagga

SEQ ID NO: 120 - DNA, 16 bp, Artificial sequence
  Synthetic oligonucleotide
gatgaaatccagaacc

SEQ ID NO: 121 - DNA, 16 bp, Artificial sequence
  Synthetic oligonucleotide
agcaaagaggtcacac

SEQ ID NO: 122 - DNA, 16 bp, Artificial sequence
  Synthetic oligonucleotide
aagttaagaaatgcct

SEQ ID NO: 123 - DNA, 16 bp, Artificial sequence
  Synthetic oligonucleotide
ccggatacagcaacac

SEQ ID NO: 124 - DNA, 16 bp, Artificial sequence
  Synthetic oligonucleotide
caacactaggtagaca

SEQ ID NO: 125 - DNA, 16 bp, Artificial sequence
  Synthetic oligonucleotide
tagcaataacagagtg

SEQ ID NO: 126 - DNA, 16 bp, Artificial sequence
  Synthetic oligonucleotide
taacctagtctttccc

SEQ ID NO: 127 - DNA, 16 bp, Artificial sequence
  Synthetic oligonucleotide
caagacaagttcttcc

SEQ ID NO: 128 - DNA, 16 bp, Artificial sequence
  Synthetic oligonucleotide
cccaaccagcgaggag

SEQ ID NO: 129 - DNA, 16 bp, Artificial sequence
  Synthetic oligonucleotide
tgctaggcggacttcc

SEQ ID NO: 130 - DNA, 16 bp, Artificial sequence
  Synthetic oligonucleotide
ccaaacccatcaacac

SEQ ID NO: 131 - DNA, 16 bp, Artificial sequence
  Synthetic oligonucleotide
aaaagcggacagaacc

SEQ ID NO: 132 - DNA, 16 bp, Artificial sequence
  Synthetic oligonucleotide
tcttagagtcccctca

SEQ ID NO: 133 - DNA, 16 bp, Artificial sequence
  Synthetic oligonucleotide
cctcaaatgtggcctg

SEQ ID NO: 134 - DNA, 16 bp, Artificial sequence
  Synthetic oligonucleotide
gaatatctgggtgaca

SEQ ID NO: 135 - DNA, 16 bp, Artificial sequence
  Synthetic oligonucleotide
agactagggagtgcac

SEQ ID NO: 136 - DNA, 16 bp, Artificial sequence
  Synthetic oligonucleotide
aataacagagtgtcag

SEQ ID NO: 137 - DNA, 16 bp, Artificial sequence
  Synthetic oligonucleotide
ctagtacctgttccaa

SEQ ID NO: 138 - DNA, 16 bp, Artificial sequence
  Synthetic oligonucleotide
atctttaattcagtta

SEQ ID NO: 139 - DNA, 16 bp, Artificial sequence
  Synthetic oligonucleotide
cacatagagacctgac

SEQ ID NO: 140 - DNA, 16 bp, Artificial sequence
  Synthetic oligonucleotide
ggtcaaaagtgccacc

SEQ ID NO: 141 - DNA, 16 bp, Artificial sequence
  Synthetic oligonucleotide
accaaaaagcggacag

SEQ ID NO: 142 - DNA, 16 bp, Artificial sequence
  Synthetic oligonucleotide
ctaccgagcttttccc

SEQ ID NO: 143 - DNA, 16 bp, Artificial sequence
  Synthetic oligonucleotide
gtcagaaaagctaacc

SEQ ID NO: 144 - DNA, 16 bp, Artificial sequence
  Synthetic oligonucleotide
gaacacggccatgctc

SEQ ID NO: 145 - DNA, 16 bp, Artificial sequence
  Synthetic oligonucleotide
atactagtcactgcca

SEQ ID NO: 146 - DNA, 16 bp, Artificial sequence
  Synthetic oligonucleotide
aggtagacatcacatc

SEQ ID NO: 147 - DNA, 16 bp, Artificial sequence
  Synthetic oligonucleotide
gactaaagataccaca

SEQ ID NO: 148 - DNA, 16 bp, Artificial sequence
  Synthetic oligonucleotide
caaacaaagcatcgcc

SEQ ID NO: 149 - DNA, 16 bp, Artificial sequence
  Synthetic oligonucleotide
catagaaaatccactg

SEQ ID NO: 150 - DNA, 16 bp, Artificial sequence
  Synthetic oligonucleotide
gcgaaagctggaggag

SEQ ID NO: 151 - DNA, 16 bp, Artificial sequence
  Synthetic oligonucleotide
agcgaggaacggtgag

SEQ ID NO: 152 - DNA, 16 bp, Artificial sequence
  Synthetic oligonucleotide
ttaacagggttgtact

SEQ ID NO: 153 - DNA, 16 bp, Artificial sequence
  Synthetic oligonucleotide
accaataggtatcagt

SEQ ID NO: 154 - DNA, 16 bp, Artificial sequence
  Synthetic oligonucleotide
ccatttgggaagttct

SEQ ID NO: 155 - DNA, 16 bp, Artificial sequence
  Synthetic oligonucleotide
gacaaccatgaagtgc

SEQ ID NO: 156 - DNA, 16 bp, Artificial sequence
  Synthetic oligonucleotide
actttgagagctgacg

SEQ ID NO: 157 - DNA, 16 bp, Artificial sequence
  Synthetic oligonucleotide
ttatagtagagggtaa

SEQ ID NO: 158 - DNA, 16 bp, Artificial sequence
  Synthetic oligonucleotide
gaaaacaagcatcagt

SEQ ID NO: 159 - DNA, 16 bp, Artificial sequence
  Synthetic oligonucleotide
ccataatccggataca

SEQ ID NO: 160 - DNA, 16 bp, Artificial sequence
  Synthetic oligonucleotide
cacatagagggtgtga

SEQ ID NO: 161 - DNA, 16 bp, Artificial sequence
  Synthetic oligonucleotide
agtaagagctaggaga

SEQ ID NO: 162 - DNA, 16 bp, Artificial sequence
  Synthetic oligonucleotide
tgagaaatacagctgt

SEQ ID NO: 163 - DNA, 16 bp, Artificial sequence
  Synthetic oligonucleotide
ggtaaacatcaaggcc

SEQ ID NO: 164 - DNA, 16 bp, Artificial sequence
  Synthetic oligonucleotide
ccatagggatccagca

SEQ ID NO: 165 - DNA, 16 bp, Artificial sequence
  Synthetic oligonucleotide
gaagatcccagagggt

SEQ ID NO: 166 - DNA, 16 bp, Artificial sequence
  Synthetic oligonucleotide
acaaccatgaagtgca

SEQ ID NO: 167 - DNA, 16 bp, Artificial sequence
  Synthetic oligonucleotide
aaggttgagaagtggc

SEQ ID NO: 168 - DNA, 16 bp, Artificial sequence
  Synthetic oligonucleotide
ccacaaggtgctcaga

SEQ ID NO: 169 - DNA, 16 bp, Artificial sequence
  Synthetic oligonucleotide
atagagacctgacggg

SEQ ID NO: 170 - DNA, 16 bp, Artificial sequence
  Synthetic oligonucleotide
caccaccaaaaagcgg

SEQ ID NO: 171 - DNA, 16 bp, Artificial sequence
  Synthetic oligonucleotide
taaagataccacatgt

SEQ ID NO: 172 - DNA, 16 bp, Artificial sequence
  Synthetic oligonucleotide
atagtagagggtaaga

SEQ ID NO: 173 - DNA, 16 bp, Artificial sequence
  Synthetic oligonucleotide
gtcaaaagtgccaccc

SEQ ID NO: 174 - DNA, 16 bp, Artificial sequence
  Synthetic oligonucleotide
ctgatagaaaccggaa

SEQ ID NO: 175 - DNA, 16 bp, Artificial sequence
  Synthetic oligonucleotide
gtaaacatcaaggccc

SEQ ID NO: 176 - DNA, 16 bp, Artificial sequence
  Synthetic oligonucleotide
ataacagagtgtcaga

SEQ ID NO: 177 - DNA, 16 bp, Artificial sequence
  Synthetic oligonucleotide
atagagggtaaacatc

SEQ ID NO: 178 - DNA, 16 bp, Artificial sequence
  Synthetic oligonucleotide
acagaacctcagagcg

SEQ ID NO: 179 - DNA, 16 bp, Artificial sequence
  Synthetic oligonucleotide
tgtttaacagggttgt

SEQ ID NO: 180 - DNA, 16 bp, Artificial sequence
  Synthetic oligonucleotide
gctaagcaaagaggtc

SEQ ID NO: 181 - DNA, 16 bp, Artificial sequence
  Synthetic oligonucleotide
cagcatcgattacaca

SEQ ID NO: 182 - DNA, 16 bp, Artificial sequence
  Synthetic oligonucleotide
gatattggtcagtaga

SEQ ID NO: 183 - DNA, 16 bp, Artificial sequence
  Synthetic oligonucleotide
gatacctcagtctcag

SEQ ID NO: 184 - DNA, 16 bp, Artificial sequence
  Synthetic oligonucleotide
gataccacatgtattc

SEQ ID NO: 185 - DNA, 16 bp, Artificial sequence
  Synthetic oligonucleotide
gaggagatactagtca

SEQ ID NO: 186 - DNA, 16 bp, Artificial sequence
  Synthetic oligonucleotide
agcaataacagagtgt

SEQ ID NO: 187 - DNA, 16 bp, Artificial sequence
  Synthetic oligonucleotide
aacctaacctagtctt

SEQ ID NO: 188 - DNA, 16 bp, Artificial sequence
  Synthetic oligonucleotide
tactccaagaacaagt

SEQ ID NO: 189 - DNA, 16 bp, Artificial sequence
  Synthetic oligonucleotide
aaaagtgactgaaccc

SEQ ID NO: 190 - DNA, 16 bp, Artificial sequence
  Synthetic oligonucleotide
cagttaaaaagtgact

SEQ ID NO: 191 - DNA, 16 bp, Artificial sequence
  Synthetic oligonucleotide
ggacaaattccagcat

SEQ ID NO: 192 - DNA, 16 bp, Artificial sequence
  Synthetic oligonucleotide
tagaaaatccactgtc

SEQ ID NO: 193 - DNA, 16 bp, Artificial sequence
  Synthetic oligonucleotide
tgaaatccagaacctc

SEQ ID NO: 194 - DNA, 16 bp, Artificial sequence
  Synthetic oligonucleotide
cataatccggatacag

SEQ ID NO: 195 - DNA, 16 bp, Artificial sequence
  Synthetic oligonucleotide
aaaagctaaccattga

SEQ ID NO: 196 - DNA, 16 bp, Artificial sequence
  Synthetic oligonucleotide
aagcgagaatgactag

SEQ ID NO: 197 - DNA, 16 bp, Artificial sequence
  Synthetic oligonucleotide
tgacaagacaagttct

SEQ ID NO: 198 - DNA, 16 bp, Artificial sequence
  Synthetic oligonucleotide
aaaacaagcatcagtc

SEQ ID NO: 199 - DNA, 16 bp, Artificial sequence
  Synthetic oligonucleotide
agccttagggaggcgc

SEQ ID NO: 200 - DNA, 16 bp, Artificial sequence
  Synthetic oligonucleotide
gagctaggagaaggca

SEQ ID NO: 201 - DNA, 16 bp, Artificial sequence
  Synthetic oligonucleotide
gccagaatgatacaag

SEQ ID NO: 202 - DNA, 16 bp, Artificial sequence
  Synthetic oligonucleotide
cgaaagctggaggagc

SEQ ID NO: 203 - DNA, 16 bp, Artificial sequence
  Synthetic oligonucleotide
ccaaccagcgaggagg

SEQ ID NO: 204 - DNA, 16 bp, Artificial sequence
  Synthetic oligonucleotide
tcaaaagcccagcttc

SEQ ID NO: 205 - DNA, 16 bp, Artificial sequence
  Synthetic oligonucleotide
ccagatagagggtaaa

SEQ ID NO: 206 - DNA, 16 bp, Artificial sequence
  Synthetic oligonucleotide
gctaaccattgaacct

SEQ ID NO: 207 - DNA, 16 bp, Artificial sequence
  Synthetic oligonucleotide
ccaataggtatcagtt

SEQ ID NO: 208 - DNA, 16 bp, Artificial sequence
  Synthetic oligonucleotide
agcccgagcacataga

SEQ ID NO: 209 - DNA, 16 bp, Artificial sequence
  Synthetic oligonucleotide
aagaactactatattc

SEQ ID NO: 210 - DNA, 16 bp, Artificial sequence
  Synthetic oligonucleotide
ccaaaaagcggacaga

SEQ ID NO: 211 - DNA, 16 bp, Artificial sequence
  Synthetic oligonucleotide
cttaaagcccctgagg

SEQ ID NO: 212 - DNA, 16 bp, Artificial sequence
  Synthetic oligonucleotide
gcttagcttaaagccc

SEQ ID NO: 213 - DNA, 16 bp, Artificial sequence
  Synthetic oligonucleotide
gagacgaagcgagaat

SEQ ID NO: 214 - DNA, 16 bp, Artificial sequence
  Synthetic oligonucleotide
gccttatagtagaggg

SEQ ID NO: 215 - DNA, 16 bp, Artificial sequence
  Synthetic oligonucleotide
gctgtaacaactggtg

SEQ ID NO: 216 - DNA, 16 bp, Artificial sequence
  Synthetic oligonucleotide
ctattgatggtgccat

SEQ ID NO: 217 - DNA, 16 bp, Artificial sequence
  Synthetic oligonucleotide
atggagaatatctggg

SEQ ID NO: 218 - DNA, 16 bp, Artificial sequence
  Synthetic oligonucleotide
caaacccatcaacact

SEQ ID NO: 219 - DNA, 16 bp, Artificial sequence
  Synthetic oligonucleotide
caatatctgtgaggat

SEQ ID NO: 220 - DNA, 16 bp, Artificial sequence
  Synthetic oligonucleotide
accaaatgctgtccta

SEQ ID NO: 221 - DNA, 16 bp, Artificial sequence
  Synthetic oligonucleotide
ctacttacaccgccgt

SEQ ID NO: 222 - DNA, 16 bp, Artificial sequence
  Synthetic oligonucleotide
tctttaattcagttac

SEQ ID NO: 223 - DNA, 16 bp, Artificial sequence
  Synthetic oligonucleotide
ccccaatcatctccta

SEQ ID NO: 224 - DNA, 16 bp, Artificial sequence
  Synthetic oligonucleotide
ctatattcttagagtc

SEQ ID NO: 225 - DNA, 16 bp, Artificial sequence
  Synthetic oligonucleotide
gagcataggcccgatg

SEQ ID NO: 226 - DNA, 16 bp, Artificial sequence
  Synthetic oligonucleotide
gactaggcaggctgag

SEQ ID NO: 227 - DNA, 16 bp, Artificial sequence
  Synthetic oligonucleotide
accattagatttgtta

SEQ ID NO: 228 - DNA, 16 bp, Artificial sequence
  Synthetic oligonucleotide
gcaaagaggtcacacc

SEQ ID NO: 229 - DNA, 16 bp, Artificial sequence
  Synthetic oligonucleotide
taacagggttgtactg

SEQ ID NO: 230 - DNA, 16 bp, Artificial sequence
  Synthetic oligonucleotide
tgcgataactggccac

SEQ ID NO: 231 - DNA, 16 bp, Artificial sequence
  Synthetic oligonucleotide
aacaaagcatcgccga

SEQ ID NO: 232 - DNA, 16 bp, Artificial sequence
  Synthetic oligonucleotide
atctcaagaactcttg

SEQ ID NO: 233 - DNA, 16 bp, Artificial sequence
  Synthetic oligonucleotide
gctacgggaactcaga

SEQ ID NO: 234 - DNA, 16 bp, Artificial sequence
  Synthetic oligonucleotide
gaggaacggtgagatc

SEQ ID NO: 235 - DNA, 16 bp, Artificial sequence
  Synthetic oligonucleotide
aacactaggtagacat

SEQ ID NO: 236 - DNA, 16 bp, Artificial sequence
  Synthetic oligonucleotide
agttaagaaatgccta

SEQ ID NO: 237 - DNA, 16 bp, Artificial sequence
  Synthetic oligonucleotide
cgccatagaaaatcca

SEQ ID NO: 238 - DNA, 16 bp, Artificial sequence
  Synthetic oligonucleotide
taagcagagactgact

SEQ ID NO: 239 - DNA, 16 bp, Artificial sequence
  Synthetic oligonucleotide
ctcaaatgtggcctgc

SEQ ID NO: 240 - DNA, 16 bp, Artificial sequence
  Synthetic oligonucleotide
gacgagttcaaaggtc

SEQ ID NO: 241 - DNA, 16 bp, Artificial sequence
  Synthetic oligonucleotide
agttaaaaagtgactg

SEQ ID NO: 242 - DNA, 16 bp, Artificial sequence
  Synthetic oligonucleotide
ccagactaaagatacc

SEQ ID NO: 243 - DNA, 16 bp, Artificial sequence
  Synthetic oligonucleotide
agatactagtcactgc

SEQ ID NO: 244 - DNA, 16 bp, Artificial sequence
  Synthetic oligonucleotide
cagatagagggtaaac

SEQ ID NO: 245 - DNA, 16 bp, Artificial sequence
  Synthetic oligonucleotide
atcttaccacgcccaa

SEQ ID NO: 246 - DNA, 16 bp, Artificial sequence
  Synthetic oligonucleotide
cctcagagcggatcgc

SEQ ID NO: 247 - DNA, 16 bp, Artificial sequence
  Synthetic oligonucleotide
actcttgaagatgtgt

SEQ ID NO: 248 - DNA, 16 bp, Artificial sequence
  Synthetic oligonucleotide
caaaaagcggacagaa

SEQ ID NO: 249 - DNA, 16 bp, Artificial sequence
  Synthetic oligonucleotide
ggtaagagggcattcg

SEQ ID NO: 250 - DNA, 16 bp, Artificial sequence
  Synthetic oligonucleotide
atcaaacaaagcatcg

SEQ ID NO: 251 - DNA, 16 bp, Artificial sequence
  Synthetic oligonucleotide
aaagataccacatgta

SEQ ID NO: 252 - DNA, 16 bp, Artificial sequence
  Synthetic oligonucleotide
taaacatcaaggcccc

SEQ ID NO: 253 - DNA, 16 bp, Artificial sequence
  Synthetic oligonucleotide
agagggtaaacatcaa

SEQ ID NO: 254 - DNA, 16 bp, Artificial sequence
  Synthetic oligonucleotide
agcacatagagacctg

SEQ ID NO: 255 - DNA, 16 bp, Artificial sequence
  Synthetic oligonucleotide
gcagaggttaactttc

SEQ ID NO: 256 - DNA, 16 bp, Artificial sequence
  Synthetic oligonucleotide
ttaattcagttactgg

SEQ ID NO: 257 - DNA, 16 bp, Artificial sequence
  Synthetic oligonucleotide
tattcttagagtcccc

SEQ ID NO: 258 - DNA, 16 bp, Artificial sequence
  Synthetic oligonucleotide
tagcttaaagcccctg

SEQ ID NO: 259 - DNA, 16 bp, Artificial sequence
  Synthetic oligonucleotide
aacaagcatcagtcac

SEQ ID NO: 260 - DNA, 16 bp, Artificial sequence
  Synthetic oligonucleotide
aaagctaaccattgaa

SEQ ID NO: 261 - DNA, 16 bp, Artificial sequence
  Synthetic oligonucleotide
gacaagacaagttctt

SEQ ID NO: 262 - DNA, 16 bp, Artificial sequence
  Synthetic oligonucleotide
agcgagaatgactagg

SEQ ID NO: 263 - DNA, 16 bp, Artificial sequence
  Synthetic oligonucleotide
gcggaagcggttgaca

SEQ ID NO: 264 - DNA, 16 bp, Artificial sequence
  Synthetic oligonucleotide
ctaagcaaagaggtca

SEQ ID NO: 265 - DNA, 16 bp, Artificial sequence
  Synthetic oligonucleotide
acttacaccgccgtgg

SEQ ID NO: 266 - DNA, 16 bp, Artificial sequence
  Synthetic oligonucleotide
gaggaaggcttacatt

SEQ ID NO: 267 - DNA, 16 bp, Artificial sequence
  Synthetic oligonucleotide
agaactactatattct

SEQ ID NO: 268 - DNA, 16 bp, Artificial sequence
  Synthetic oligonucleotide
atattggtcagtagaa

SEQ ID NO: 269 - DNA, 16 bp, Artificial sequence
  Synthetic oligonucleotide
gcagagactgacttcc

SEQ ID NO: 270 - DNA, 16 bp, Artificial sequence
  Synthetic oligonucleotide
gaaaatccactgtcag

SEQ ID NO: 271 - DNA, 16 bp, Artificial sequence
  Synthetic oligonucleotide
aaacccatcaacacta

SEQ ID NO: 272 - DNA, 16 bp, Artificial sequence
  Synthetic oligonucleotide
cctgagcggaacacgg

SEQ ID NO: 273 - DNA, 16 bp, Artificial sequence
  Synthetic oligonucleotide
caatggatgacacacg

SEQ ID NO: 274 - DNA, 16 bp, Artificial sequence
  Synthetic oligonucleotide
ccattagatttgttag

SEQ ID NO: 275 - DNA, 16 bp, Artificial sequence
  Synthetic oligonucleotide
gcaataacagagtgtc

SEQ ID NO: 276 - DNA, 16 bp, Artificial sequence
  Synthetic oligonucleotide
tcaaaagtgccaccca

SEQ ID NO: 277 - DNA, 16 bp, Artificial sequence
  Synthetic oligonucleotide
ccagaacctcaaatgt

SEQ ID NO: 278 - DNA, 16 bp, Artificial sequence
  Synthetic oligonucleotide
ctccaagaacaagtta

SEQ ID NO: 279 - DNA, 16 bp, Artificial sequence
  Synthetic oligonucleotide
gacaaattccagcatg

SEQ ID NO: 280 - DNA, 16 bp, Artificial sequence
  Synthetic oligonucleotide
gacgaagcgagaatga

SEQ ID NO: 281 - DNA, 16 bp, Artificial sequence
  Synthetic oligonucleotide
gtttaacagggttgta

SEQ ID NO: 282 - DNA, 16 bp, Artificial sequence
  Synthetic oligonucleotide
ccaccaaaaagcggac

SEQ ID NO: 283 - DNA, 16 bp, Artificial sequence
  Synthetic oligonucleotide
ctcctaaagacaggta

SEQ ID NO: 284 - DNA, 16 bp, Artificial sequence
  Synthetic oligonucleotide
ctaaccattgaacctt

SEQ ID NO: 285 - DNA, 16 bp, Artificial sequence
  Synthetic oligonucleotide
ctgtagaagattccac

SEQ ID NO: 286 - DNA, 16 bp, Artificial sequence
  Synthetic oligonucleotide
gcataggcccgatggc

SEQ ID NO: 287 - DNA, 16 bp, Artificial sequence
  Synthetic oligonucleotide
caaagcatcgccgaga

SEQ ID NO: 288 - DNA, 16 bp, Artificial sequence
  Synthetic oligonucleotide
ctgtaacaactggtgg

SEQ ID NO: 289 - DNA, 16 bp, Artificial sequence
  Synthetic oligonucleotide
ccttatagtagagggt

SEQ ID NO: 290 - DNA, 16 bp, Artificial sequence
  Synthetic oligonucleotide
aataggtatcagttgg

SEQ ID NO: 291 - DNA, 16 bp, Artificial sequence
  Synthetic oligonucleotide
ggagtaagagctagga

SEQ ID NO: 292 - DNA, 16 bp, Artificial sequence
  Synthetic oligonucleotide
aaagaggtcacaccat

SEQ ID NO: 293 - DNA, 16 bp, Artificial sequence
  Synthetic oligonucleotide
ccagaatgatacaaga

SEQ ID NO: 294 - DNA, 16 bp, Artificial sequence
  Synthetic oligonucleotide
agctaggagaaggcat

SEQ ID NO: 295 - DNA, 16 bp, Artificial sequence
  Synthetic oligonucleotide
aaccggccatctgcga

SEQ ID NO: 296 - DNA, 16 bp, Artificial sequence
  Synthetic oligonucleotide
cccttagcaataacag

SEQ ID NO: 297 - DNA, 16 bp, Artificial sequence
  Synthetic oligonucleotide
gactcaagagggacct

SEQ ID NO: 298 - DNA, 16 bp, Artificial sequence
  Synthetic oligonucleotide
gccatagaaaatccac

SEQ ID NO: 299 - DNA, 16 bp, Artificial sequence
  Synthetic oligonucleotide
caaccatgaagtgcag

SEQ ID NO: 300 - DNA, 16 bp, Artificial sequence
  Synthetic oligonucleotide
atcgattacacacaga

SEQ ID NO: 301 - DNA, 16 bp, Artificial sequence
  Synthetic oligonucleotide
cctaacctagtctttc

SEQ ID NO: 302 - DNA, 16 bp, Artificial sequence
  Synthetic oligonucleotide
gagaatatctgggtga

SEQ ID NO: 303 - DNA, 16 bp, Artificial sequence
  Synthetic oligonucleotide
tgcacatagagggtgt

SEQ ID NO: 304 - DNA, 16 bp, Artificial sequence
  Synthetic oligonucleotide
cgataactggccacac

SEQ ID NO: 305 - DNA, 16 bp, Artificial sequence
  Synthetic oligonucleotide
ataatccggatacagc

SEQ ID NO: 306 - DNA, 16 bp, Artificial sequence
  Synthetic oligonucleotide
ctgcatacggctggag

SEQ ID NO: 307 - DNA, 16 bp, Artificial sequence
  Synthetic oligonucleotide
ttaaagcccctgaggc

SEQ ID NO: 308 - DNA, 16 bp, Artificial sequence
  Synthetic oligonucleotide
gttaagaaatgcctac

SEQ ID NO: 309 - DNA, 16 bp, Artificial sequence
  Synthetic oligonucleotide
taacagagtgtcagac

SEQ ID NO: 310 - DNA, 16 bp, Artificial sequence
  Synthetic oligonucleotide
ccaaatgctgtcctag

SEQ ID NO: 311 - DNA, 16 bp, Artificial sequence
  Synthetic oligonucleotide
ccatagcactgaaatc

SEQ ID NO: 312 - DNA, 16 bp, Artificial sequence
  Synthetic oligonucleotide
ccatttatcttcagcg

SEQ ID NO: 313 - DNA, 16 bp, Artificial sequence
  Synthetic oligonucleotide
acactaggtagacatc

SEQ ID NO: 314 - DNA, 16 bp, Artificial sequence
  Synthetic oligonucleotide
taccatactctgccag

SEQ ID NO: 315 - DNA, 16 bp, Artificial sequence
  Synthetic oligonucleotide
tcagaaccacttctgg

SEQ ID NO: 316 - DNA, 22 bp, Artificial sequence
  Primer
tgcctctagcacacaactgtga

SEQ ID NO: 317 - DNA, 22 bp, Artificial sequence
  Primer
gcaggaatgatttggaaaggaa

SEQ ID NO: 318 - DNA, 23 bp, Artificial sequence
  Probe
cgttttgtggatcagcgcctcca

SEQ ID NO: 319 - DNA, 20 bp, Artificial sequence
  Primer
tggattcccgttcgagtacg

SEQ ID NO: 320 - DNA, 19 bp, Artificial sequence
  Primer
tcagctggatagcgacatc

SEQ ID NO: 321 - DNA, 20 bp, Artificial sequence
  Probe
aagcgagggctccgacccga

SEQ ID NO: 322 - DNA, 21 bp, Artificial sequence
  Primer
tcatggaaagcctctgtggat

SEQ ID NO: 323 - DNA, 16 bp, Artificial sequence
  Primer
gcggcccgtgatgaga

SEQ ID NO: 324 - DNA, 24 bp, Artificial sequence
  Probe
cccacaagtcccagaaccgcagtg

SEQ ID NO: 325 - DNA, 18 bp, Artificial sequence
  Primer
aaacggaagcgcatcgaa

SEQ ID NO: 326 - DNA, 20 bp, Artificial sequence
  Primer
gggactggcgagccttagtt

SEQ ID NO: 327 - DNA, 23 bp, Artificial sequence
  Probe
ccatccgtggccagatcctgtcc

SEQ ID NO: 328 - DNA, 24 bp, Artificial sequence
  Primer
caggttctgtccctttcactcact

SEQ ID NO: 329 - DNA, 23 bp, Artificial sequence
  Primer
ctgtgctcatggtgtcttttctg

SEQ ID NO: 330 - DNA, 21 bp, Artificial sequence
  Probe
cccaaggcgccacatctccct

SEQ ID NO: 331 - DNA, 16 bp, Artificial sequence
  Synthetic oligonucleotide
cgccgataaggtacac